(19) United States
(12) Patent Application Publication
(10) Pub. No.: US 2024/0110177 A1
(43) Pub. Date: Apr. 4, 2024

(54) A SCREENING PLATFORM FOR ADAR-RECRUITING GUIDE RNAS

(71) Applicant: The Board of Trustees of the Leland Stanford Junior University, Stanford, CA (US)

(72) Inventors: Jin Billy Li, Stanford, CA (US); Inga Jarmoskaite, Stanford, CA (US); Paul Vogel, Stanford, CA (US)

(21) Appl. No.: 18/249,597

(22) PCT Filed: Oct. 21, 2021
(86) PCT No.: PCT/US2021/056064

§ 371 Date: Apr. 19, 2023

Related U.S. Application Data
(60) Provisional application No. 63/094,614, filed on Oct. 21, 2020.

(51) Int. Cl. C12N 15/11

(52) U.S. Cl. CPC C12N 15/111; C12N 2320/11

(57) ABSTRACT

The present invention relates to methods for identifying guide RNAs for use in site-directed RNA editing. In particular, the present invention relates to a high-throughput screening method for identifying guide RNAs effective for site directed A-to-I RNA editing, and methods of use for the identified guide RNAs.

### STATEMENT REGARDING RELATED APPLICATIONS

This application claims priority to U.S. Non-Provisional Patent Application. No. 63/094,614, filed Oct. 21, 2020, the entire contents of which are incorporated herein by reference.

### FIELD

The present invention relates to methods for identifying guide RNAs for use in site-directed RNA editing. In particular, the present invention relates to a high-throughput screening method for identifying guide RNAs (gRNAs) effective for site directed A-to-I RNA editing, and methods of use for the identified guide RNAs. Additionally, the invention relates to guide RNA sequences that have been identified by this screening approach to he superior in the repair of the premature W402X stop codon in the human IDUA (alpha-L-iduronidase) transcript.

### BACKGROUND

Site-directed RNA editing is a new technology for manipulating genetic information on the RNA level. This is accomplished by small guide RNAs that recruit the endogenous RNA editing enzymes, ADARs (adenosine deaminases acting on RNA), or engineered ADAR fusion proteins, to user-defined target RNAs, thereby enabling the conversion of specified adenosine residues to inosines (A-to-I editing). Since inosine is biochemically interpreted as guanosine, site-directed A-to-I RNA editing has the potential to manipulate RNA and protein function for therapeutic and bioengineering purposes.

Current ADAR guide RNA designs feature an anti sense domain of variable length that is complementary to the target sequence, and an optional recruitment domain for ADAR binding. Only a small number of ADAR guide designs have been tested so far, with disparate degrees of success achieved in editing of different targets, and uniform design principles are yet to be established. Given the up to 100% editing efficiency of ADAR's diverse natural RNA targets, there appears to be great potential for further optimizing ADAR guide RNAs. However, such optimization efforts have been hampered by the lack of suitable high-throughput approaches for rapid screening of guide candidates. Accordingly, what is needed are methods for high-throughput screening of guide RNA candidates for use in A-to-I RNA editing.

### SUMMARY

In some aspects, provided herein are fusion constructs. In some embodiments, provided herein is a fusion construct comprising a target sequence and a guide RNA sequence. In some embodiments, the guide RNA sequence comprises an antisense domain that is substantially complementary or perfectly complementary to the target sequence. In some embodiments, the guide RNA sequence further comprises a recruitment domain that recruits endogenous adenosine deaminases acting on RNA (ADARs) and/or engineered ADAR fusion proteins. in some embodiments, the recruitment domain comprises a first strand and a second strand that are substantially complementary or perfectly complementary to each other.

In some embodiments, the fusion construct further comprises a loop sequence, such that the construct forms a stern loop secondary structure. The loop sequence may comprise any suitable number of nucleotides. In some embodiments, the loop sequence comprises 3-50 nucleotides. In some embodiments, the loop sequence comprises 5 nucleotides. In some embodiments, the loop sequence comprises a nucleotide sequence set forth in Table 1. In some embodiments, the antisense domain and the target sequence are linked by the loop sequence. In some embodiments, the first strand and the second strand of the recruitment domain are linked by the loop sequence.

In some embodiments, the guide RNA sequence comprises one or more mutations in the antisense domain that disrupt base pairing between the antisense domain and the target sequence in at least one nucleotide location. In some embodiments, the guide RNA sequence comprises one or more mutations in the first strand and/or the second strand of the recruitment domain that disrupt base pairing between the first strand and the second strand in at least one nucleotide location. In some embodiments, the first strand comprises a nucleotide sequence having at least 50% sequence identity to SEQ ID NO: 3. For example, in some embodiments the first strand comprises a nucleotide sequence having at least 80% sequence identity to SEQ ID NO: 3. In some embodiments, the first strand comprises a nucleotide sequence set forth in Table 2. In some embodiments, the second strand comprises a nucleotide sequence having at least 50% sequence identity to SEQ ID NO: 4. For example, in some embodiments the second strand comprises a nucleotide sequence having at least 80% sequence identity to SEQ ID NO: 4. In some embodiments, the second strand comprises a nucleotide sequence set forth in Table 3.

In some embodiments, the target sequence is derived from the human IDUA gene. In some embodiments, the target sequence comprises a nucleotide sequence having at least 80% sequence identity to GAGCAGCUCUAGGCCGAA (SEQ ID NO: 1). In some embodiments, the nucleotide at position 11 relative to SEQ ID NO: 1 is an adenine (A). In some embodiments, the antisense domain comprises a nucleotide sequence having at least 50% sequence identity to SEQ ID NO: 2. In some embodiments, the antisense domain comprises a sequence set forth in Table 5 or Table 6.

In some aspects, provided herein are vectors. In some embodiments, provided herein is a vector comprising a fusion construct described herein. The fusion constructs and vectors described herein may be used in a high-throughput screening method for selecting guide RNAs for use in site-directed RNA editing

In some aspects, provided herein are high-throughput screening methods. In some embodiments, provided herein is a high-throughput screening method for selecting guide RNAs for use in site-directed RNA editing. In some embodiments, the method comprises generating a plurality of fusion constructs, each fusion construct comprising a target sequence and a guide RNA sequence. In some embodiments, the guide RNA sequence comprises an antisense domain that is substantially complementary or perfectly complementary to the target sequence.

In some embodiments, the method further comprises expressing each of the plurality of fusion constructs in a distinct population of cells. In some embodiments, the method further comprises determining whether a fusion construct induces one or more modifications in nucleic acid isolated from the population of cells expressing the fusion construct. In some embodiments, the cells express endogenous adenosine deaminases acting on RNA (ADARs) and/or at least one engineered ADAR fusion protein.

In some embodiments of the methods described herein, the guide RNA sequence further comprises a recruitment domain that recruits endogenous adenosine deaminases acting on RNA (ADARs) and/or engineered ADAR fusion proteins. In some embodiments, the recruitment domain comprises a first strand and a second strand that are substantially complementary or perfectly complementary to each other.

In some embodiments of the methods described herein, the fusion construct further comprises a loop sequence, such that the construct forms a stern loop secondary structure. In some embodiments, the loop sequence comprises 3-50 nucleotides. For example, in some embodiments the loop sequence comprises 5 nucleotides. In some embodiments, the loop sequence comprises a nucleotide sequence set forth in Table 1. In some embodiments, the antisense domain and the target sequence are linked by the loop sequence. In some embodiments, the first strand and the second strand of the recruitment domain are linked by the loop sequence.

In some embodiments, the guide RNA sequence comprises one or more mutations in the antisense domain that disrupt base pairing between the antisense domain and the target sequence in at least one nucleotide location. In some embodiments, the guide RNA sequence comprises one or more mutations in the first strand and/or the second strand of the recruitment domain that disrupt base pairing between the first strand and the second strand in at least one nucleotide location. In some embodiments, the first strand comprises a nucleotide sequence having at least 50% sequence identity to SEQ ID NO: 3. For example, in some embodiments the first strand comprises a nucleotide sequence having at least 80% sequence identity to SEQ ID NO: 3. In some embodiments, the first strand comprises a nucleotide sequence set forth in Table 2. In some embodiments, the second strand comprises a nucleotide sequence having at least 50% sequence identity to SEQ ID NO: 4. For example, in some embodiments the second strand comprises a nucleotide sequence having at least 80% sequence identity to SEQ ID NO: 4. In some embodiments, the second strand comprises a nucleotide sequence set forth in Table 3.

In some embodiments, the target sequence is derived from a gene for which site-directed A-to-I RNA editing is desired. In some embodiments, the gene comprises a point mutation, wherein the point mutation is a G to A point mutation, a T to A point mutation, or a C to A point mutation. In some embodiments, the point mutation is associated with development of a disease or condition in a subject expressing the gene. In some embodiments, the point mutation is present in the target sequence.

In some embodiments, determining whether a fusion construct induces one or more modifications in nucleic acid isolated from the population of cells expressing the fusion construct comprises sequencing the isolated nucleic acid. In some embodiments, the isolated nucleic acid comprises RNA. In some embodiments, the one or more modifications in nucleic acid isolated from the population of cells comprises a correction of the point mutation initially present in the target sequence. In some embodiments, correction of the point mutation indicates that the guide RNA sequence effectively induces site-directed RNA editing.

In some embodiments, the target sequence comprises a nucleotide sequence having at least 80% sequence identity to GAGCAGCUCUAGGCCGAA (SEQ ID NO: 1). In some embodiments, the nucleotide at position 11 relative to SEQ ID NO: 1 is an adenine (A). In come embodiments, the antisense domain comprises a nucleotide sequence having at least 50% sequence identity to SEQ ID NO: 2. In some embodiments, the antisense domain comprises a sequence set forth in Table 5 or Table 6.

In some embodiments of the methods described herein, wherein the method identifies one or more optimized features of the guide RNA sequence that enable the guide RNA sequence to induce one or more modifications in nucleic acid isolated from the population of cells expressing the fusion construct. For example, the optimized features may be selected from the antisense domain, the loop sequence, and the recruitment domain, if present in the guide RNA.

In some aspects, provided herein are methods for site-directed RNA editing. In some embodiments, provided herein is a method for site-directed RNA editing, the method comprising selecting a guide RNA by the methods described herein, and delivering a construct comprising the guide RNA to a cell or a subject. For example, the method for site-directed RNA editing may comprise selecting a guide RNA by a high-throughput screening method described herein, and delivering a construct comprising the selected guide RNA to a cell or a subject. In some embodiments, the cell is a mammalian cell. In some embodiments, the subject is a mammal.

In some aspects, provided herein are guide RNAs. In some embodiments, provided herein are guide RNAs for use in site-directed RNA editing. In some embodiments, provided herein is a guide RNA for use in site-directed RNA editing, wherein the guide RNA comprises an antisense domain that is substantially complementary or perfectly complementary to a target gene sequence. In some embodiments, the guide RNA comprises a recruitment domain that recruits endogenous adenosine deaminases acting on RNA (ADARs) and/or engineered ADAR fusion proteins. In some embodiments, the recruitment domain comprises a first strand and a second strand that are substantially complementary or perfectly complementary to each other. In some embodiments, the first strand and the second strand are linked by a loop sequence. In some embodiments, the loop sequence comprises 3-50 nucleotides. For example, in some embodiments the loop sequence comprises 5 nucleotides. In some embodiments, the loop sequence comprises a nucleotide sequence set forth in Table 1.

In some embodiments, the first strand comprises a nucleotide sequence having at least 50% sequence identity to SEQ ID NO: 3. For example, in some embodiments the first strand comprises a nucleotide sequence having at least 80% sequence identity to SEQ ID NO: 3. In some embodiments, the first strand comprises a nucleotide sequence set forth in Table 2. In some embodiments, the second strand comprises a nucleotide sequence having at least 50% sequence identity to SEQ ID NO: 4. For example, in some embodiments the second strand comprises a nucleotide sequence having at least 80% sequence identity to SEQ ID NO: 4. In some embodiments, the second strand comprises a nucleotide sequence set forth in Table 3.

In some embodiments, the target gene sequence is present within a portion of the human IDUA gene containing a W402X substitution mutation. In some embodiments, the target gene sequence comprises SEQ ID NO: 5. In some embodiments, the antisense domain comprises a nucleotide sequence having at least 50% sequence identity to SEQ ID NO: 2. In some embodiments, the antisense domain comprises a sequence set forth in Table 5 or Table 6. In some embodiments, the guide RNA may be used in a method of treating Hurler syndrome.

Other aspects and embodiments of the disclosure will be apparent in light of the following detailed description and accompanying figures.

### BRIEF DESCRIPTION OF THE DRAWINGS

FIG. 1 is a schematic showing adenosine-to-inosine (A-to-I) editing in RNA. Since inosine is recognized as guanosine by the cellular machinery, A-to-I editing formally introduces A-to-G point mutations that can influence RNA and protein function.

FIG. 2 shows design of endogenous ADAR-recruiting guide RNAs (gRNAs). ADARs, composed of a deaminase domain (ADAR-D) and multiple dsRNA-binding domains (dsRBDs), edit the R/G site located in a hairpin structure of GRIA2 pre-mRNA (left panel). Fusing a part of the hairpin structure (55 nt) to an antisense sequence (18-40 nt), complementary to a user-defined sequence, results in the generation of a gRNA which directs the ADAR enzyme to the target adenosine. The hairpin functions as ADAR-recruiting part enabling the interaction with the dsRBDs while the hybrid of the gRNA antisense domain and the target RNA is recognized by the deaminase domain catalyzing editing at the target site. To recruit ADAR, R/G gRNAs are either expressed from a plasmid or applied as a chemically modified antisense oligonucleotide (ASO).

FIG. 3 is a schematic showing an overview of the method for optimizing gRNA sequence. In order to achieve high editing yields, a screening platform is used in mammalian cells to find gRNA sequences that maximize RNA editing.

FIG. 4A-4E. Potential applications of therapeutic A-to-I RNA editing. (A) 12 out of the 20 canonical amino acids and all three stop codons can be changed by A-to-I editing. (B,C) Site-directed A-to-I RNA editing of codons, encoding phosphorylation sites (B) or other functional important sites (C), might be used to modulate the function of proteins who's in- or overactivation improves disease outcomes. (D) Inhibition of translation can be achieved by the editing of the start codon, which might be an option to downregulate disease-causing proteins. (E) A-to-I RNA editing can correct pathogenic G-to-A point mutations.

FIG. 5. Pathogenic G-to-A point mutation causing Hurler syndrome. gRNA sequences may be screened for their ability to edit human IDUA W402X (red underlined A). Letters beneath the IDUA mRNA sequence represent the one letter code of the encoded amino acids and the premature stop codon (X).

FIG. 6. Overview of the screening platform. Target RNA/gRNA fusion constructs may be expressed in ADAR-Flp-In T-REx cells via plasmid lipofection. After RNA isolation, target RNA/gRNA cDNA may be generated for next-generation sequencing (NGS). The use of different indexes will allow the concurrent analysis of multiple experiments. A computational pipeline may be established for the determination of the induced editing yield at the target adenosine and at surrounding off-site adenosines for every single gRNA sequence.

FIG. 7. Overview on libraries for optimizing the gRNA antisense domain. To identify both the induced editing at the target site and the corresponding gRNA by the platform, the target sequence (black) is fused to the gRNA (antisense domain: ADAR-recruiting part: red). Here, the IDUA W402X mRNA sequence containing the pathogenic point mutation (red underlined A) is shown as the target.

FIG. 8. Overview on libraries for optimizing the ADAR-recruiting part.

FIG. 9A-9G. ASO library prototypes. (A) Target-guide fusion construct based on the previously validated guide design ‘v9.4’³² with a single T-to-C base substitution at position 40 in the loop region. The target sequence is the region surrounding the pathogenic W402X mutation in the human IDUA gene (hIDUA). The targeted A residue is shown in yellow. (B, C) Editing levels, determined by Sanger sequencing 24 h after plasmid transfection into Flp-In T-REx 293 cells without (B) or with (C) inducible expression of ADAR1 p150. In the absence of p150 induction, editing is mediated by endogenous ADAR proteins. Identical results (50% editing) were obtained in Flp-In T-REx cells with and without stably integrated ADAR1 p150 in the absence of Dox induction. (D) Modified fusion prototype consisting of only target and anti sense sequences linked by a short loop (i.e., no recruitment domain). The target sequence is the region surrounding the pathogenic W402X mutation in hIDUA, extended on the 3′-end to provide a binding site for ADAR's double-stranded RNA binding domains (dsRBDs). Two mismatches were introduced in the anti sense strand (positions 54 and 58) to mimic the structure of the GRIA2 R/G site. (E) Editing of the construct in panel (D) 24 h after transfection into ADAR1 p150 Flp-In T-REx 293 cells without Dox induction; editing was saturated with Dox induction. (F) Split design, in which the target and antisense regions are separated by the EGFP coding sequence. (G) Editing of the construct in panel (F) 24 h after transfection into ADAR1 p150 Flp-In T-REx 293 cells, induced with 10 ng/mL Dox; no editing was observed in the absence of Dox.

FIG. 10A-10B. Cloning constructs. (A) Plasmid map and schematic representation of the pcDNA5-based cloning vector used for the IDUA W402X screen. Asterisk denotes the stop codon; in the case of IDUA W402X, an additional stop codon is present in the unedited target sequence and is removed by editing, RE, restriction enzyme cleavage site. (B) Alternative cloning vector, used for the split design shown in FIG. 9F. For a given target, the target sequence only needs to be cloned once, and new guide libraries can be easily inserted using restriction sites RE 1 & 2.

FIG. 11A-11B. Sequences of the custom inserts into the pcDNA5 vector. (A) Sequence of the linked target/guide construct (FIG. 10A), shown here for IDUA W402X. (B) Sequence of the split construct, where the target (top) and guide (bottom) sequences are separated by the EGFP coding sequence (FIG. 10B). Additional restriction enzyme sites have been introduced to enable insertion of a full guide sequence (using HpaI or PacI and AvrII or BstBI) or exchange of the antisense domain only (using Bsu36I and HpaI or PacI). To include the Bsu36I site, the sequence identity of three base-pairs in the recruitment domain was altered while maintaining the original structure. This sequence change did not decrease the editing level relative to the split construct that maintained the original recruitment domain sequence (FIG. 9F), with editing levels of 33% and 28% detected in the presence of the recruitment domain with and without the Bsu36I restriction site, respectively.

FIG. 12. PCR assembly of the target/guide fusion construct with a randomized antisense region.

FIG. 13. Sequence details of primers used for the PCR assembly of the IDUA W402X ASO library. To ensure efficient amplification of the highly structured assembled template, the outer primers should be distant from the target/guide duplex.

FIG. 14. Reverse transcription and sequencing library preparation. UMI, unique molecular identifier, consisting of 15 random nucleotides. The UMI allows to uniquely distinguish each reverse transcript in subsequent quantification, eliminating the effects of PCR bias and sequencing errors^{71, 72} Sequence elements colored in cyan correspond to standard Illumina adapter sequences. Here, long flanking regions were used to ensure that Illumina bridge amplification is not affected by the stable hairpin structure. FIG. 15. Sequence details of the library construct and primers shown in FIG. 14.

FIG. 16, top panel shows an exemplary hairpin construct (e.g., comprising a recruitment domain, a target sequence, and a guide antisense oligonucleotide) targeting IDUA W402X, which may be generated by methods described herein, in particular as described in Example 3. A library of antisense domain mutants was generated by randomizing the antisense sequence. The histogram shows the predicted distribution of antisense variants with different numbers of mutations given 18% degeneracy at each antisense position.

FIG. 17 shows an exemplary workflow, as described herein and in particular in Example 3.

FIG. 18 is a bar graph showing that approx. 1% of antisense oligonucleotide variants increase editing at the target site compared to the prototype construct.

FIG. 19 shows antisense oligonucleotide variants containing mutations that enhance editing, as identified in the pilot screen.

FIG. 20 shows validation of a highly edited variant identified in the screen (bottom left) by Sanger sequencing (bottom right); the prototype sequence (top left) and the corresponding editing level (top right) are also shown.

FIG. 21 shows examples of recruitment domain (based on the GRIA2 R/G RNA) mutations that enhance editing by restoring one of the base-pairs disrupted in the original recruitment domain. The prototype is shown at the top, with three single mutants that enhance editing shown below.

FIG. 22 shows base enrichments at each position of the recruitment domain terminal loop. Enrichment was calculated based on the top 10% edited variants (n=102 relative to the entire loop library (n=1015).

FIG. 23 shows the numbering of nucleotide positions used to indicate sequence changes in Tables 2-6.

FIG. 24 shows the additive effect of combining an optimized loop sequence in the recruitment domain and a beneficial mismatch in the antisense region. The constructs shown in the figure were individually cloned and transfected into FlpIn T-REx cells expressing only endogenous ADAR protein. The editing level was determined by Sanger sequencing.

FIG. 25 shows the sequence of the human IDUA gene. Note that this sequence does not contain the W402X mutation seen in patients with Hurler Syndrome.

### DETAILED DESCRIPTION OF THE INVENTION

The present disclosure is directed to methods for identifying, guide RNAs for use in site-directed RNA editing. In particular, the present invention relates to a high-throughput screening method for identifying guide RNAs effective for site directed A-to-I RNA editing.

### 1. Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0 6,1, 6.2, 6,3, 6.4, 6.5, 6.6, 6,7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclature used in connection with, and techniques of, cell and tissue culture, biochemistry, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine, N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As used herein, the term “artificial” refers to compositions and systems that are designed or prepared by humankind, and are not naturally occurring. For example, an artificial peptide or nucleic acid is one comprising a non-natural sequence (e.g., a nucleic acid or a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K); and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidontimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

The term “amino acid analog” refers to a natural or unnatural amino acid Where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0,1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

The term “adenosine deaminases acting on RNA” or “ADARs” are used herein to refer to a class of enzymes that naturally catalyze A-to-I editing of sites within double-stranded RNA (dsRNA) regions of the transcriptome of higher organisms. ADARs can play important roles in the regulation of protein function, RNA splicing, immunity and RNA interference.

The term “ADAR fusions” as used herein refers to engineered enzymes that comprise an ADAR deaminase domain and a domain which is able to bind a guide RNA.

The term “donor nucleic acid molecule” refers to a nucleotide sequence that is inserted into the target DNA (e.g., genomic DNA). As described above the donor DNA may include, for example, a gene or part of a gene, a sequence encoding a tag or localization sequence, or a regulating element. The donor nucleic acid molecule may be of any length. In some embodiments, the donor nucleic acid molecule is between 10 and 10,000 nucleotides in length. For example, between about 100 and 5,000 nucleotides in length, between about 200 and 2,000 nucleotides in length, between about 500 and 1,000 nucleotides in length, between about 500 and 5,000 nucleotides in length, between about 1,000 and 5,000 nucleotides in length, or between about 1,000 and 10,000 nucleotides in length.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine (C), thymine (T), and uracil (U), and adenine (A) and guanine (G), respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97: 5633-5638 (2000), incorporated herein by reference), cyclohexenyl nucleic acids (see Wang, J. Am. Chem. Soc., 122: 8595-8602 (2000), incorporated herein by reference), and/or a ribozyme. Hence, the term “nucleic acid” or “nucleic acid sequence” may also encompass a chain comprising non natural nucleotides, modified nucleotides, and/or non-nucleotide building blocks that can exhibit the same function as natural nucleotides (i.e., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The term “linker,” as used herein, refers to a bond (e.g., covalent bond), chemical group, or a molecule linking two molecules or moieties, e.g., two domains of a fusion protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20-30, 40-50, 50-60, 60-70, 70-80, 80-90, 90-100, 100-150, or 150-200 amino acids in length. Longer or shorter linkers are also contemplated herein.

The term “mutation,” as used herein, refers to a substitution of a residue within a sequence, e.g., a. nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to, the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “guide RNA,” as used herein refers to a nucleic acid designed to be complementary to the “target sequence”. The terms “target RNA sequence;” “target nucleic acid,” “target sequence,” and “target site” are used interchangeably herein to refer to a polynucleotide (nucleic acid, gene, chromosome, genome, etc.) to which a guide RNA sequence is designed to have complementarity. Typically, the gRNA and target RNA form a dsRNA duplex structure with a central A:C mismatch at the targeted site to induce efficient and precise editing by the ADAR deaminase domain.

In some embodiments, the guide RNAs (also referred to herein as ASOs) described herein comprise two components: an antisense domain and a recruitment domain. The terms “antisense domain” and “antisense sequence” are used interchangeably herein. The antisense domain (i.e., antisense sequence) of the gRNA binds to the target RNA. The recruitment domain (also referred to herein as the ADAR-recruiting part), enables the interaction with the ADAR or ADAR fusion protein. In some embodiments, the guide RNAs described herein comprise only the antisense domain (i.e., lack a recruitment domain). In some embodiments, the guide RNAs described herein may be optimized for RNA editing. For example, a guide RNA may contain one or more mutations to optimize RNA editing. Suitable locations for the mutations and types of mutations are described herein.

The target sequence and guide sequence need not exhibit complete complementarity, provided that there is sufficient complementarity to cause hybridization. Suitable gRNA:RNA binding conditions include physiological conditions normally present in a cell. Other suitable binding conditions (e.g., conditions in a cell-free system) are known in the art; see, e.g., Sambrook, referenced herein and incorporated by reference.

The target RNA sequence may be a gene product. The term “gene product,” as used herein, refers to any biochemical product resulting from expression of a gene. Gene products may be RNA or protein. RNA gene products include non-coding RNA, such as tRNA, rRN, micro RNA (miRNA), and small interfering RNA (siRNA), and coding RNA, such as messenger RNA (mRNA).

A “vector” or “expression vector” is a replicon, such as plasmid, phage, virus, or cosmid, to which another DNA segment, e.g. an “insert,” may be attached or incorporated so as to bring about the replication of the attached segment in a cell. For example, the “insert” may be a construct as described herein. For example, the “insert” may be a construct comprising a target sequence and a guide RNA sequence as described herein.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally-occurring mutants can be isolated; these are identified by the fact that they have altered characteristics when compared to the wild-type gene or gene product.

### 2. Fusion Constructs

In some embodiments, provided herein are fusion constructs. In some embodiments, provided herein are fusion constructs comprising a guide RNA sequence and a target sequence. The fusion constructs provided herein find use in various methods, including methods of high-throughput screening for selecting guide RNAs for use in site-directed RNA editing.

In some embodiments, the fusion construct possesses a stem loop secondary structure. The terms “hairpin,” “hairpin loop,” “stem loop,” and/or “loop” are used interchangeably herein to refer to a structure formed in a single stranded oligonucleotide when sequences within the single strand which are complementary when read in opposite directions base pair to form a region whose conformation resembles a hairpin or loop.

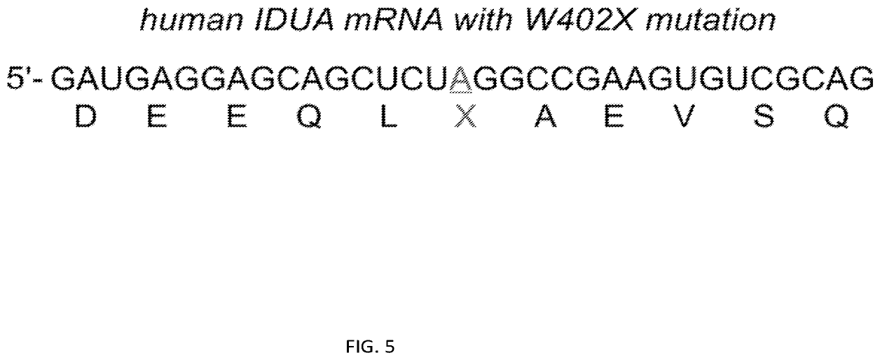

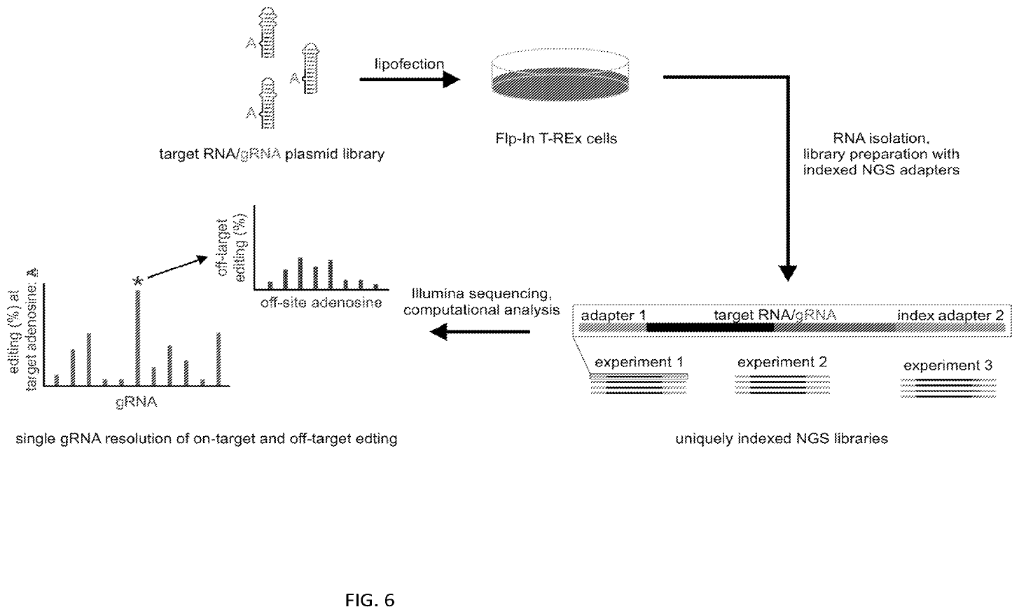

In some embodiments, the fusion construct comprises a target sequence. The target sequence is selected based upon the gene of interest (i.e., the gene for which site-directed A-to-I RNA editing is desired). In some embodiments, the target sequence comprises a mutated sequence. For example, the target sequence may comprise a nucleotide sequence possessing one or more mutations, wherein said one or more mutations result in a disease phenotype. In some embodiments, the gene of interest is IDUA. The sequence of the human IDUA gene is shown in FIG. 5. In some embodiments, the gene of interest is IDUA and the target sequence comprises or is derived from a portion of the sequence of IDUA containing a G to A mutation that results in a premature IDUA W402X stop codon causing Hurler Syndrome. However, this is not intended to be a limiting example and the constructs described herein may comprise any suitable target sequence to be used in high-throughput methods for selecting guide RNA sequences with optimized RNA editing capability for any desired gene.

In some embodiments, the target sequence comprises a nucleotide sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to GAGCAGCUCUAGGCCGAA (SEQ ID NO: 1), provided that nucleotide at position 11 relative to SEQ ID NO: 1 is an adenine (A).

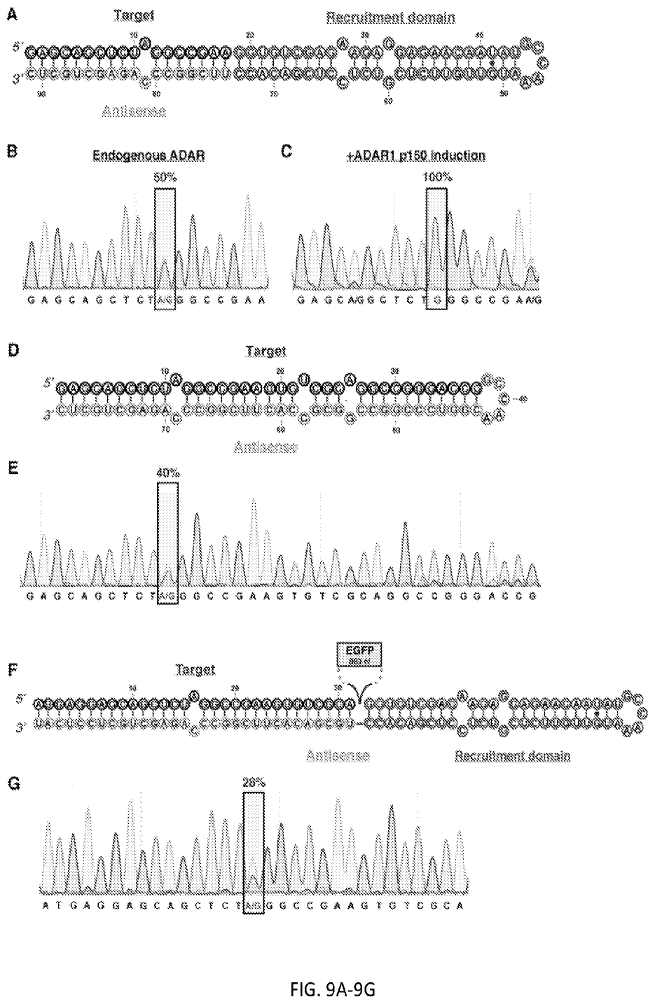

in some embodiments, the guide RNA sequence comprises an antisense domain. The antisense domain of the gRNA binds to the target RNA. Accordingly, selection of the sequence of the antisense domain depends on the sequence of the target RNA of interest (i.e., the desired RNA to be edited). The antisense domain may comprise any suitable number of nucleotides. In some embodiments, the antisense domain comprises 10-50 nucleotides. For example, in some embodiments the antisense domain comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, the antisense domain comprises more than 50 nucleotides. in some embodiments, the antisense domain comprises 10-30 nucleotides. In some embodiments, the antisense domain comprises 15-25 nucleotides. In some embodiments, the length of the antisense domain depends on whether the guide RNA additionally comprises a recruitment domain. For example, guide RNA sequences lacking a recruitment domain may contain antisense domains of longer length compared to guide RNA sequences containing both a recruitment domain and an antisense domain. This concept is exemplified in FIG. 9. For example, as shown in FIG. 9A the length of the antisense domain is 18 nucleotides in a guide RNA comprising a recruitment domain, whereas in FIG. 9D the length of the antisense domain is 37 nucleotides in a guide RNA lacking a recruitment domain.

In some embodiments, the guide RNA described herein lacks a recruitment domain. For example, in some embodiments the guide RNA comprises a target sequence and an antisense domain, and does not comprise a recruitment domain. In some embodiments, the target sequence and the antisense domain are linked by a loop structure, such that the construct forms a stem-loop secondary structure. The loop structure may comprise any suitable number of nucleotides. In some embodiments, the loop structure comprises 3-50 nucleotides. In some embodiments, the loop structure comprises 3-50 nucleotides, 3- 45 nucleotides, 3-40 nucleotides, 3-35 nucleotides, 3-30 nucleotides, 3-25 nucleotides, 3-20 nucleotides, 3-15 nucleotides, 3-10 nucleotides, or 3-7 nucleotides. In some embodiments, the loop structure is a pentaloop (i.e., comprises 5 nucleotides). In some embodiments, the loop structure comprises a sequence set forth in Table 1. In some embodiments, the loop structure comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, OR SEQ ID NO: 18.

In some embodiments, the guide RNA comprises an antisense domain and a recruitment domain. The guide RNA sequence may be optimized for RNA editing, such as by making one or more mutations in the antisense domain and/or recruitment domain as described herein.

In some embodiments, the antisense domain is intended to target a portion of the human IDUA gene. However, the high-throughput sequencing methods described herein may be applied to any suitable target to identify optimized gRNAs for site directed editing of any desired gene. In some embodiments, the anti sense domain is substantially complementary to the target sequence. Accordingly, nucleotides within the antisense domain base pair with corresponding nucleotides on the target sequence, thus forming the secondary structure of the construct (i.e., the stem loop structure of the construct). The base pairing need not be 100%. For example, in some embodiments one or more nucleotides in the antisense domain do not base pair with the nucleotide in the corresponding location in the target sequence. In some embodiments, the antisense domain comprises one or more mutations that disrupt perfect complementarity (i.e., disrupt base pairing). For example, the antisense domain may comprise one or more mutations that disrupt base pairing with the target sequence, which may result in mismatches within the stem of the stem loop structure. In some embodiments, the antisense domain comprises a nucleotide sequence having at least 50% sequence identity to UUCGGCCCAGAGCUGCUC (SEQ ID NO: 2). For example, the antisense domain may comprise a nucleotide sequence having at least 50%, at least 60%, at least 70%, at least 75%, at least 80%, at least 85%, at least 90%. at least 91%, at least 92%, at least 93%, at least 94%, at least 95%, at least 96%, at least 97%, at least 98%, at least 99%, or 100% sequence identity to SEQ ID NO: 2. In some embodiments, the nucleotide at position 8 relative to SEQ ID NO: 2 (i.e., the position opposite from the target adenosine residue in the target strand) is a cytidine. The nucleotides 3′ of position 8 (i.e., 3′ of the cytidine at position 8) are denoted herein as “−” followed by the number of nucleotides away from position 8, whereas the nucleotides 5′ of position 8 are denoted herein as “+” followed by the number of nucleotides away from position 8. In some embodiments, the antisense domain comprises a nucleotide sequence as shown in Table 4. In some embodiments, the antisense domain comprises a nucleotide sequence of SEQ ID NO: 195.

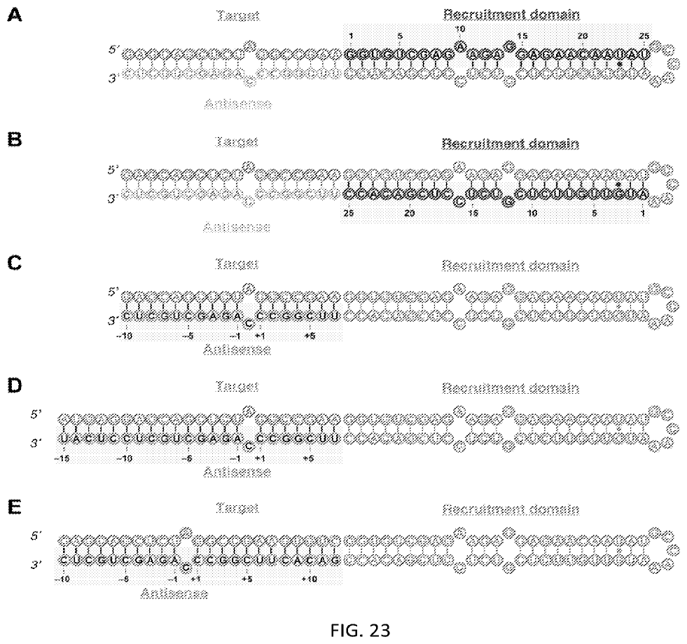

In some embodiments, the antisense domain possesses more than 18 nucleotides. For example, the antisense domain may comprise additional nucleotides in addition to those present in the sequence having at least 50% identity to SEQ ID NO: 2. Such additional oligonucleotides may be present at the 3′ end or the 5′ end of the antisense domain. Exemplary such antisense domains are highlighted in FIG. 23D and FIG. 23E, each of which show additional nucleotides (e.g., 5 nucleotides in addition to the 18-nt antisense domain used in the original construct) added to the 3′ end or the 5′ end of an antisense strand. In some embodiments, the antisense domain comprises a sequence as shown in Table 5 or Table 6.

In some embodiments, the antisense domain comprises a sequence shown in Table 5. In some embodiments, the antisense domain comprises a nucleotide sequence of SEQ ID NO: 202. In some embodiments, the antisense domain comprises a nucleotide sequence shown in Table 6. In some embodiments, the antisense domain comprises a nucleotide sequence of SEQ ID NO: 303. In some embodiments, the antisense domain comprises a nucleotide sequence of SEQ ID NO: 304.

In some embodiments, the guide RNA sequence comprises a recruitment domain. The recruitment domain (also referred to herein as the ADAR-recruiting part), facilitates the interaction with the ADAR or ADAR fusion protein. The recruitment domain is configured to bind (i.e., recruit) one or more ADAR proteins or fusions thereof. For example, the recruitment domain may be configured to recruit an ADAR1, an ADAR2 protein or a fusion thereof. In some embodiments, the recruitment domain recruits at least an ADAR2 protein. The recruitment domain may comprise any suitable number of nucleotides. For example, the recruitment domain may comprise 15-100 nucleotides. In some embodiments, the recruitment domain comprises about 15, about 20, about 25, about 30, about 35, about 40, about 45, about 50, about 55, about 60, about 65, about 70, about 75, about 80, about 85, about 90, about 95, or about 100 nucleotides. In some embodiments, the recruitment domain is part of a construct that possesses a stem-loop secondary structure. In some embodiments, the recruitment domain forms a part of a stem loop structure. In some embodiments, the loop portion of the stem loop structure consists of 5 nucleotides (i.e., pentaloop).

In some embodiments, the recruitment domain is based upon the sequence of an endogenous (i.e., naturally occuring) ADAR target. The recruitment domain may possess one or more modifications compared to the endogenous ADAR target, which may enhance ADAR recruitment or interactions. For example, the recruitment domain may be based upon the sequence of the GRIA2 R/G site, an endogenous target for ADAR2.

In some embodiments, the recruitment domain comprises a first strand (i.e., a 5′ strand) and a second strand (i.e., a 3′ strand) connected by a loop structure (also referred to herein as a loop sequence). The first strand and the second strand exhibit complementary base pairing, thus assisting in the formation of the stem loop structure of the construct. In some embodiments, this base pairing is disrupted by one or more mutations within the first strand and/or the second strand of the recruitment domain. In some embodiments, an unmodified recruitment domain refers to a recruitment domain that exhibits base pairing with no disruptions (i.e., perfect complementarity), whereas a mutated recruitment domain refers to a domain comprising one or more mutations in the first strand or the second strand that disrupt base pairing. In other words, an unmodified recruitment domain comprises a first strand with perfect complementarity to a second strand, whereas a mutated recruitment domain comprises a first strand and a second strand with substantial (i.e., at least 60%), but not perfect complementarity.

In some embodiments the recruitment domain comprises a first strand and a second strand connected by a loop structure. The loop structure may comprise any suitable number of nucleotides. In some embodiments, the loop structure comprises 3-50 nucleotides. In some embodiments, the loop structure comprises 3-50 nucleotides, 3-45 nucleotides, 3-40 nucleotides, 3-35 nucleotides, 3-30 nucleotides, 3-25 nucleotides, 3-20 nucleotides, 3-15 nucleotides, 3-10 nucleotides, or 3-7 nucleotides. In some embodiments, the loop structure is a pentaloop structure. Suitable sequences of a pentaloop structure are shown in Table 1. Any of the sequences shown in Table 1 may be used for a fusion construct as described herein. In some embodiments, the loop structure comprises SEQ ED NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, OR SEQ ID NO: 18.

In some embodiments, the first strand (i.e., the 5′ strand) comprises a nucleotide sequence having at least 50% sequence identity to GGUGUCGAGAAGAGGAGAACAAUAU (SEQ ID NO: 3). For example, the first strand may comprise a nucleotide sequence having at least 50%, at least 60%, at least 70%, at least 75%, at least 80%, at least 85%, at least 90%, at least 91%, at least 92%, at least 93%. at least 94%, at least 95%, at least 96%, at least 97%, at least 98%, at least 99%, or 100% sequence identity to SEQ ID NO: 3. In some embodiments, the first strand (i.e., the 5′ strand) comprises a sequence as shown in Table 2. In some embodiments, the first strand comprises a nucleotide sequence of SEQ ID NO: 108. In some embodiments, the first strand comprises a nucleotide sequence of SEQ ID NO: 109.

In some embodiments, the second strand comprises nucleotide sequence having at least 50% sequence identity to AUGUUGUUCUCGUCUCCUCGACACC (SEQ ID NO: 4). For example, the second strand may comprise a nucleotide sequence having at least 50%, at least 60%, at least 70%, at least 75%, at least 80%. at least 85%, at least 90%, at least 91%, at least 92%, at least 93%, at least 94%, at least 95%, at least 96%, at least 97%, at least 98%, at least 99%, or 100% sequence identity to SEQ ID NO: 4. In some embodiments, the second strand (i.e., 3′ strand) comprises a sequence as shown in Table 3. In some embodiments, the second strand comprises a nucleotide sequence of SEQ ID NO: 144. In some embodiments, the second strand comprises a nucleotide sequence of SEQ ID NO: 145. In some embodiments, the second strand comprises a nucleotide sequence of SEQ ID NO: 146.

In some embodiments, the first strand comprises a nucleotide sequence having at least 50% sequence identity to SEQ ID NO: 3 and the second strand comprises a nucleotide sequence having at least 50% sequence identity to SEQ ID NO: 4, and the first and second strand are connected by a loop structure. In some embodiments, the loop structure is a pentaloop structure. Suitable sequences of a pentaloop structure are shown in Table 1. Any of the sequences shown in Table 1 may be used for a fusion construct as described herein. In some embodiments, the loop structure comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, OR SEQ ID NO: 18.

In some embodiments, the fusion construct comprises a combination of mutations. The combination of mutations may be in one or more regions within the construct. For example, the fusion construct may comprise multiple mutations in the guide RNA. For example, the construct may comprise one or more mutations within the antisense domain (i.e., one or more mutations that disrupt a given base pairing with a corresponding nucleotide in the target sequence) of the guide RNA and one or more mutations within the recruitment domain of the guide RNA (i.e., one or more mutations that disrupt or restore base pairing between the first strand and the second strand of the recruitment domain). For example, in some embodiments the construct comprises an antisense domain as set forth in Table 4, Table 5, or Table 6, and a loop sequence set forth in Table 1. In some embodiments, the construct comprises an antisense domain as set forth in Table 4, Table 5, or Table 6, and a recruitment domain comprising a first sequence as set forth in Table 2 and/or a second sequence as set forth in Table 3. In some embodiments, the construct comprises an antisense domain as set forth in Table 4, Table 5, or Table 6, a loop sequence as set forth in Table 1, and recruitment domain comprising a first sequence as set forth in Table 2 and/or a second sequence as set forth in Table 3.

In some embodiments, the fusion construct comprises one or more components in addition to the guide RNA sequence and the target sequence. For example, the fusion construct may additionally comprise one or more components to facilitate determination of whether the construct is effectively expressed in a cell of interest. For example, the fusion construct may additionally comprise sequences encoding a fluorescent protein, which enables visualization of whether a construct is expressed in a cell of interest. In some embodiments, the fusion construct comprises intervening sequences between the guide RNA sequence and the target sequence. Such intervening sequences may comprise any suitable number of nucleic acids. For example, the fusion construct may comprise a sequence encoding a fluorescent protein, which may assist in determining that the construct is expressed in a cell of interest. Such an. embodiment is shown, for example, in FIG. 9F.

### 3. High-Throughput Screening Methods

Great efforts have been made to develop tools that enable precise manipulation of genetic information. Besides various applications in the life sciences, these tools have a great potential to be used for the treatment of diseases, especially those where classical therapeutic approaches, using antibodies or small molecules, would fail. One approach to precisely changing genetic information is the targeted manipulation of the genome. The CRISPR-Cas system has made genome engineering a mainstream method that is widely used in basic research to study gene function in vitro and in vivo.^{1,2} Intense efforts are currently being undertaken to translate this technology to the clinic. However, the way to its therapeutic use remains challenging, which is highlighted by recent reports showing that the CRISPR-Cas system can induce cell cycle arrest³, cell death⁴ or an immune response⁵⁻⁷. The fact that changes introduced into DNA persist permanently is both a blessing and a curse at the same time. On the one hand, genome engineering offers a chance for permanent cure of challenging diseases. On the other hand, this is accompanied with enormous safety risks since potentially harmful off-target mutations, occurring as unintentional by-products, might be stably installed in the genome.

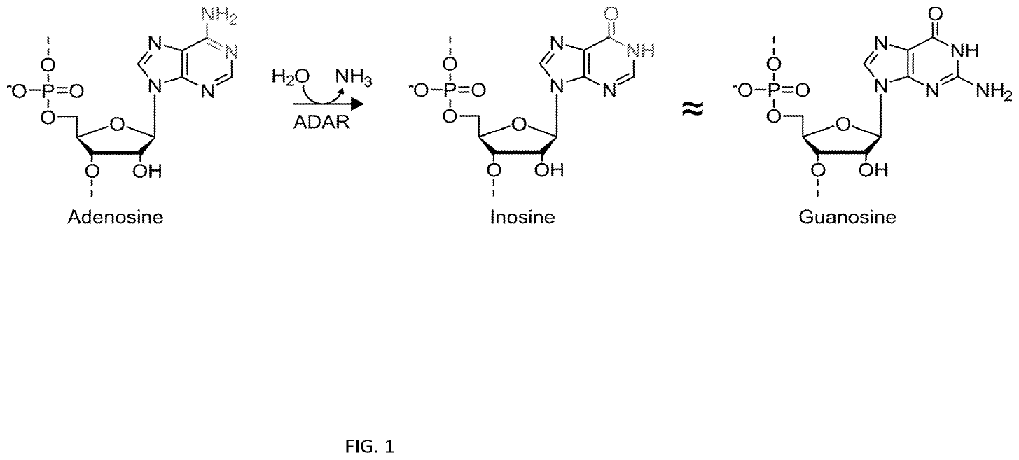

The manipulation of genetic information without the safety concerns that are associated with genome engineering might be achieved by tools enabling transcriptome engineering, as changes made in RNA are transient. The reversibility of RNA modification offers the opportunity to temporarily manipulate essential biological processes, such as cell signaling or inflammation, whose permanent alteration would otherwise have serious consequences. Additionally, the tunability of introducing a change in RNA (potentially from 0% to 100%) allows the precise regulation of the biological outcome. In the recent years, several tools have been developed enabling the site-specific conversion of adenosine to inosine (FIG. 1) in target RNAs, known as site-directed A-to-I RNA editing.^{8,9} Since inosine is biochemically interpreted as guanosine by the cellular machinery, A-to-I editing formally introduces A-to-G point mutations in RNA, which offers the opportunity to manipulate or restore genetic information. So far, all tools for site-specific A-to-I editing use the catalytic activity of adenosine deaminases acting on RNA (ADARs).^{8,9} These enzymes naturally catalyze A-to-I editing at millions of sites within double-stranded RNA (dsRNA) regions of the transcriptome of higher organisms and play important roles in the regulation of protein function, RNA splicing, immunity and RNA interference.¹⁰⁻¹⁴ To direct the catalytic activity of ADARs to a specific site within the transcriptome, there are several strategies using either engineered ADAR fusions or endogenous ADAR enzymes.

ADARs share common structural features which include multiple dsRNA-binding domains (dsRBDs) at the N-terminus and a C-terminal deaminase domain. The dsRBDs largely contribute to the promiscuity of ADARs as they enable the binding to various dsRNA structures. To engineer a specific editing machine (i.e., ADAR fusion protein), the dsRBDs are removed, and the ADAR deaminase domain is fused to a protein domain allowing the interaction with a guide RNA (gRNA), leading to the formation of a deaminase-gRNA complex. By applying simple base pairing rules, the gRNA directs the engineered deaminase to any chosen target RNA. Typically, the gRNA and target RNA form a dsRNA duplex structure with a central A:C mismatch at the targeted site to induce efficient and precise editing by the deaminase domain^{8,9}

Several deaminase-gRNA complexes have been engineered whose assemblies are mediated by the MS2-MCP^{15,16}, CRISPR-Cas13^{17,70}, λN-boxB¹⁸⁻²⁰ or SNAP-tag²¹⁻²³ system. For example, the ADAR fusion protein may comprise an ADAR deaminase domain fused to a Cas enzyme. For example, ADAR fusion proteins have been shown to carry out C-to-U editing when fused with Cas13b¹⁷.

To perform site-directed RNA editing, the engineered ADAR fusion and the gRNA have to be ectopically introduced into the cell. Under optimized conditions, ADAR-fusion-gRNA complexes can edit transcripts with almost quantitative yield.^{17,20,23} However, it is recurrently found that efficient editing typically comes along with numerous off-target editing all over the transcriptome (up to several tens of thousands of off target sites), which arises from the high levels of the engineered ADAR fusions in the cell after ectopic expression.^{16,17,23,27}

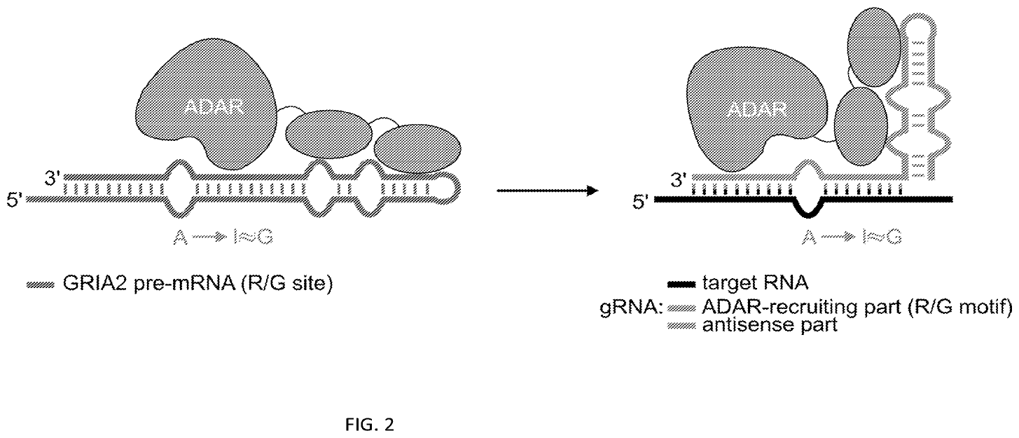

One possibility to perform site-directed RNA editing without the risk of off-target editing associated with the ectopic expression of a deaminase, is by harnessing endogenous ADAR enzymes. The first evidence that human ADARs can indeed be used for site-directed editing was provided by the groups of Stafforst and Fukuda.²⁸⁻³⁰ However, successful editing was still dependent on the ectopic expression of the ADAR enzymes. In those reports, ADARs were recruited towards target RNAs by plasmid-derived gRNAs containing two functional domains. The first domain, the antisense domain of the gRNA, binds to the target RNA, while the second domain, the ADAR-recruiting part, is intended to facilitate the interaction with the ADAR dsRBDs (FIG. 2). ADAR-mediated editing takes place at the target site once the target RNA and gRNA form a duplex which mimics natural dsRNA editing targets.³² Site-directed RNA editing in cell culture can be performed with endogenous ADARs.³² In contrast to the former studies, the described gRNAs were given as chemically modified antisense oligonucleotides (ASOs) instead of being expressed from a plasmid. Targeting of several endogenous transcripts with chemically modified gRNAs yielded efficient RNA editing in a wide variety of cell types. Additionally, the editing has been shown to be precise and not to disturb the natural editing homeostasis as only a few differently edited off-target sites (14 sites with significantly increased or attenuated editing) were found.³²

Endogenous ADARs require highly potent gRNAs to perform site-directed RNA editing with sufficient efficiency. However, cell culture experiments with ADAR-recruiting gRNAs with the current state-of-the art design showed that many target sites were only edited under 50%.³² Given that ADARs naturally edit sites in the human transcriptome with yields up to 100%,⁴⁶ there is still potential for improving the gRNA design for maximum site-directed RNA editing. However, rational gRNA engineering for highly selective and efficient editing within the formed target RNA/gRNA duplex remains challenging.

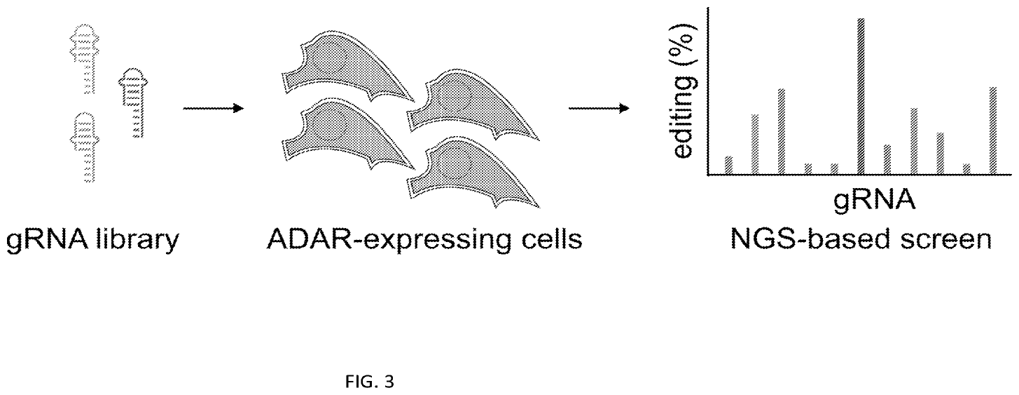

In some embodiments, provided herein are systems and methods that find use to identify, select, produce, and utilize gRNAs that maximize the RNA editing yield. The platform allows the high-throughput screening of gRNA sequences for their ability to mediate site-directed RNA editing in mammalian cells (FIG. 3). The results obtained from the screen provide a better understanding of effective site-directed RNA editing with ADARs and engineered ADAR fusions. The platform provides a powerful approach to optimize the gRNA sequence for an individual target site. Additionally, the platform is able not only to quantify the editing yield at the target site, but at all other surrounding off-site adenosines which are located within the duplex between target RNA and gRNA. This provides an impression of how (off-site/target) editing is regulated by the duplex sequence and structure. This information is not only useful for site-directed RNA editing, but also to understand the editing outcome at known sites in the human transcriptome.

In some embodiments, provided herein is a high-throughput screening method for selecting guide RNAs for use in site-directed RNA editing. In some embodiments, the method comprises generating a plurality of fusion constructs as described herein. The fusion constructs comprise a target sequence and a guide RNA sequence as described herein. In some embodiments, the target sequence is derived from a gene for which site-directed A-to-I RNA editing is desired. For example, in some embodiments, the gene comprises a G to A point mutation, a T to A point mutation, or a C to A point mutation. In some embodiments, correction of such a mutation is desired. For example, correction of a G to A point mutation, correction of a T to A point mutation, or correction of a C to A point mutation may be desired. In some embodiments, the point mutation is associated with development of a disease or condition in a subject expressing the gene. For example, the subject may suffer from Hurler Syndrome. In some embodiments, point mutation is present in the target sequence. For example, the target sequence may contain the G to A point mutation, T to A point mutation, or C to A point mutation which causes a disease or condition in a subject expressing the gene. In some embodiments, the mutation is a G to A point mutation, and the mutation is present in the target sequence.

The methods further comprise inducing expression of the fusion construct in a suitable cell. For example, the method may further comprise transfecting cells expressing adenosine deaminases acting on RNA (ADARs) or cells expressing ADAR fusion proteins with the fusion constructs. The method further comprises determining whether a fusion construct effectively induces one or more mutations in nucleic acid isolated from the cells relative to a control. Any suitable cells expressing ADARs or ADAR fusion proteins may be used. Suitable cells include eukaryotic cells including but not limited to yeast cells, higher plant cells, animal cells, insect cells, and mammalian cells. Non-limiting examples of eukaryotic cells include simian, bovine, porcine, murine, rat, avian, reptilian and human cells.

Transfection methods may be assisted by the use of suitable cell permeabilizing agents (e.g., lipofectamine) or may be performed by other suitable techniques such as electroporation. The fusion constructs may be housed in a suitable vector prior to delivery to the cell. Suitable vectors include viral vectors (e.g., lentiviral vectors, retroviral vectors, adenovirus vectors, adeno-associated virus vectors, alphavirus vectors, etc.) and non-viral vectors (e.g., plasmids, cosmids, phages, etc.). Following achieving the desired expression of the construct within the cell, the method further comprises determining whether a given fusion construct effectively induces one or more modifications in nucleic acid isolated from the cells relative to a control. Accordingly, in some embodiments the method further comprises isolating nucleic acid from the cells. The isolated nucleic acid may be RNA.

In some embodiments, determining whether a fusion construct induces one or more modifications in nucleic acid isolated from the population of cells expressing the fusion construct comprises sequencing the isolated nucleic acid. In sonic embodiments, the one or more modifications in nucleic acid isolated from the population of cells comprises a correction of the mutation (e.g. G to A point mutation, C to A point mutation, or to A point mutation) initially present in the target sequence. For example, RNA may be isolated from the cells and sequencing may be performed to determine whether the G to A point mutation initially present in the target sequence has been corrected. For example, successful recruitment of ADARs enables modification of selected adenine residues to inosine. Since inosine is biochemically interpreted as guanosine by the cellular machinery, A-to-I editing introduces A-to-G point mutations in RNA. Accordingly, point mutations present in the target sequence, such as a G to A point mutation present in the target sequence, may be corrected. For example, the adenosine residue originally present in the target sequence may be corrected to a guanine residue. Correction of the G to A point mutation indicates that the guide RNA sequence effectively induces site-directed RNA editing (i.e., site-directed A-to-I RNA editing).

In some embodiments, the method further comprises determining whether expression of the construct effectively induced a modification in the RNA compared to a control. For example, the method may comprise determining the sequence of the isolated nucleic acid (e.g., RNA). A variety of suitable sequencing methods and technologies may be used to determine the sequence of the nucleic acid strands. For example, the sequencing method may be Sanger sequencing. As another example, the sequencing method may be a next generation sequencing technology (e.g., next generation RNA sequencing technology). The term next generation sequencing, or “NGS”, refers to a variety of sequencing techniques that permit simultaneous sequencing of millions of nucleic acid sequences, and is otherwise referred to as high-throughput sequencing or massively parallel sequencing. In some embodiments, RNA may be isolated from the cells and cDNA of the target RNA/gRNA fusions may be prepared for subsequent sequencing with NGS (such as by using a platform commercially available from Illumina). For the sequencing library preparation, NGS adapters with different indexes may be used, which allows the concurrent analysis of multiple constructs. To analyze the sequencing data, a computational pipeline may be used which enables the detection of editing levels within the target RNA sequences and the identification of the corresponding gRNAs.

In some embodiments, the methods described herein may be used to identify gRNAs comprising one or more optimized features such that a guide RNA comprising the optimized feature(s) effectively induces site-directed RNA editing. The optimized features may be selected from the antisense domain, the recruitment domain, and the loop sequence. For example, the methods described herein may be used to identify optimized antisense domains, target sequences, loop sequences, and/or recruitment domain sequences. In some embodiments, the methods described herein may be used to identify optimized antisense domains. Accordingly, such optimized antisense domains may be used in circular guide RNAs or in guide RNAs lacking a recruitment domain. For example, optimized antisense domains may be used in circular guide RNAs or in guide RNAs lacking a recruitment domain for methods of site-directed gene editing. Alternatively, optimized antisense domains may be used in combination with another optimized feature in a guide RNA, such as an optimized recruitment domain and/or an optimized loop sequence. In some embodiments, the methods described herein may be used to identify gRNAs containing an optimized recruitment domain. For example, the methods may identify gRNAs containing optimized first strand sequences and/or optimized second strand sequences for a recruitment domain. In some embodiments, the methods may identify optimized loop sequences. Accordingly, the methods described herein may be used to assist in the generation of guide RNAs containing one or more optimized features, including an optimized antisense domain, an optimized target sequence, and optimized loop sequence and/or an optimized recruitment domain sequence.

### 4. Guide RNAs and Therapeutic Methods

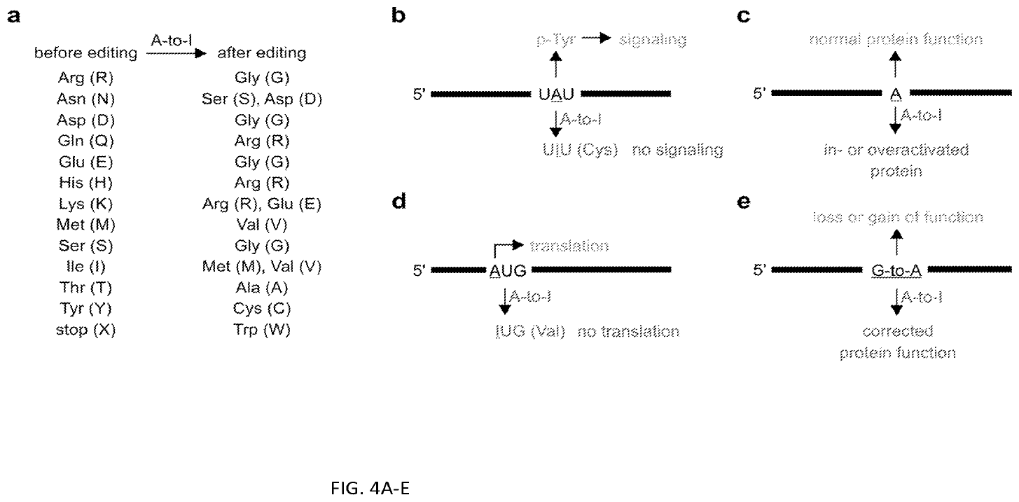

The therapeutic capability of site-directed A-to-I RNA editing results from its ability to produce a change in codon meaning by formally introducing an A-to-G point mutation. All three stop codons and 12 out the 20 canonical amino acids can he recoiled by A-to-I editing (FIG. 4A). This includes tyrosine, serine and threonine residues which typically serve as phosphorylation sites in signaling proteins (FIG. 4B). Editing of those phosphorylation sites finds use to correct aberrant signaling in diseases, such as cancer. Indeed, site-directed A-to-I editing has been successfully applied to efficiently edit the 5′-UAU triplet in STAT1 mRNA,^{23,32} coding for Y701 whose phosphorylation is essential for signal transduction.³³ Besides the recoding of amino acid residues serving for phosphorylation, A-to-I editing finds use to induce amino acid substitutions at other sites which are functionally important (FIG. 4C). This is useful to alter the function of proteins whose inactivation or overactivation has a beneficial effect in the treatment of diseases. Furthermore, inhibiting the function of disease-causing proteins is also feasible by targeting the 5′-AUG start codon, whose editing leads to a valine codon (5′-IUG), which prevents translation initiation (FIG. 4D).

A particularly appealing application of therapeutic A-to-I RNA editing is the repair of pathogenic G-to-A point mutations (FIG. 4D). According to the ClinVar database (http://www.ncbi.nlm.nih.gov/clinvar/), there are thousands of disease-causing G-to-A point mutations that modulate protein function (gain or loss of function) or alter RNA splicing. Several reports have been published indicating that site-directed A-to-I RNA editing finds use as a powerful approach in medicine to correct pathogenic G-to-A point mutations.^{16,18,20,22,32}

Site-directed A-to-I RNA editing finds use to reverse the above-described and other disease phenotypes caused by G-to-A point mutations without the safety concerns which are associated with genome engineering. In a therapeutic context, harnessing endogenous ADARs for site-directed RNA editing is promising since this approach is currently much more precise than those applying ectopically expressed engineered ADAR fusions.^{17,23,32,43} Furthermore, successful editing with endogenous ADARs requires only the administration of the gRNA as a chemically modified nucleic acid, which enormously simplifies the therapeutic application of site-directed RNA editing. Suitable modifications include, but are not limited to, 2′-O-methyl (2′-OMe), phosphorothioate (PS), 2′-O-methyl thioPACE (MSP), 2′-O-methyl-PACE (MP), 2′-fluoro RNA (2′,-F-RNA), and constrained ethyl (S-cEt). Alternatively, the gRNA can be expressed from a plasmid, e.g., with adeno-associated virus (AAV) delivery.

In some embodiments, provided herein are methods for harnessing endogenous ADARs for the correction of the premature IDUA W402X stop codon causing Hurler Syndrome (FIG. 5). Such methods may significantly benefit from highly efficient repair of the disease-causing G-to-A point mutation. Accordingly, methods for treating Hurler syndrome are preceded by optimization of gRNA using the systems and methods described herein. Subsequently to identifying optimized gRNA(s), said gRNA(s) may be used in the methods for treating disease as described herein.

In some embodiments, provided herein are methods for site-directed RNA editing. The methods comprise selecting a gRNA by a method/platform as described herein, and providing a construct comprising the guide RNA to a cell or a subject. In some embodiments, the guide RNA is a gRNA as described herein. In some embodiments, the construct may additionally comprise a targeting domain, as described herein.

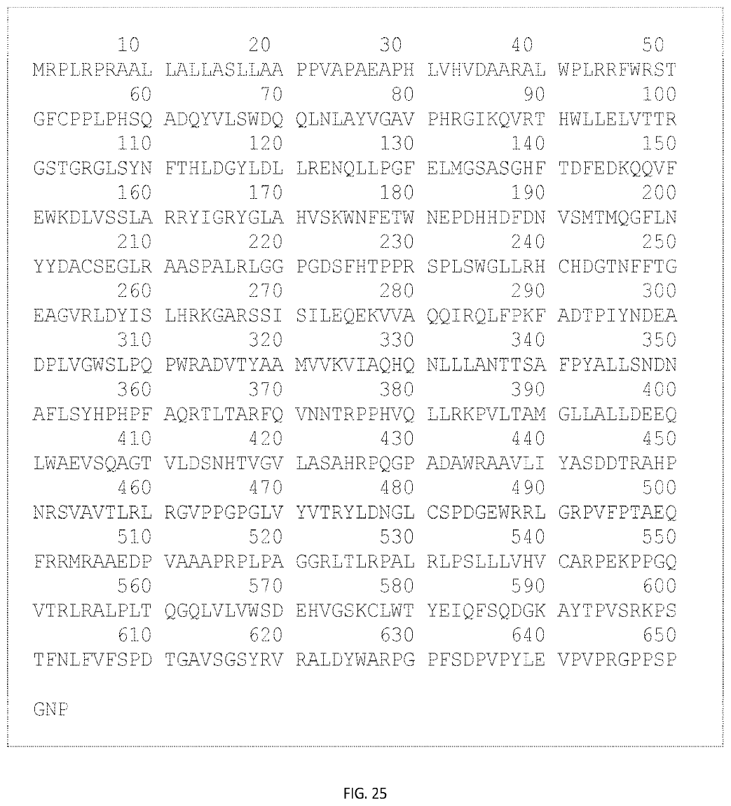

In some embodiments, provided herein are guide RNAs for use in site-directed RNA editing,. The guide RNA may be any suitable guide RNA described herein. The guide RNA may be identified using a high-throughput screening method as described herein. In some embodiments, the guide RNA comprises an antisense domain that is substantially complementary or perfectly complementary to a target gene sequence. The target gene sequence may be any gene sequence for which site-directed RNA editing is desired. In some embodiments, the target gene sequence is present within the IDUA gene. For example, the target gene sequence may be present within the human IDUA gene. The sequence of the human IDUA gene is shown in FIG. 25. As shown in FIG. 25, the amino acid at position 402 is tryptophan (W). However, a W402X mutation is seen in the gene in subjects with Hurler Syndrome. Accordingly, in some embodiments, the target gene sequence comprises the W402X mutation present in the human IDUA mRNA. The target gene sequence may comprise this W402X mutation, along with any suitable number of nucleotides in either direction of the W402X mutation. In some embodiments, the target gene sequence may comprise

(SEQ ID NO: 5)
GAUGAGGAGCAGCUCUAGGCCGAAGUGUCGCAG.

Selection of an appropriate antisense domain sequence depends on the target gene of interest. In some embodiments, the antisense domain is intended to target a portion of the human IDUA gene, however other genes of interest may be targeted. In some embodiments, the antisense domain is designed such that nucleotides within the antisense domain base pair with corresponding nucleotides on the target sequence. In some embodiments, the antisense domain is perfectly complementary to the target gene sequencing. In other embodiments, one or more nucleotides in the antisense domain are mutated such that they do not base pair with the nucleotide in the corresponding location in the target sequence (i.e., the antisense domain is substantially, but not perfectly, complementary with the target sequence). In some embodiments, the antisense domain comprises a nucleotide sequence having at least 50% sequence identity to UUCGGCCCAGAGCUGCUC (SEQ ID NO: 2). For example, the antisense domain may comprise a nucleotide sequence having at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ I NO: 2. In some embodiments, the nucleotide at position 8 relative to SEQ ID NO: 2 (i.e., the nucleotide opposite from the target adenosine within the target-antisense duplex) is a cytidine. In some embodiments, the antisense domain comprises a nucleotide sequence as shown in Table 4. The nucleotides 3′ of position 8 (i.e., 3′ of the cytidine at position 8) are denoted herein as “−” followed by the number of nucleotides away from position 8, whereas the nucleotides 5′ of position 8 are denoted herein as “+” followed by the number of nucleotides away from position 8. In some embodiments, the antisense domain comprises a nucleotide sequence set forth in SEQ ID NO: 195.

In some embodiments, the antisense domain possesses more than 18 nucleotides. For example, the antisense domain may comprise additional nucleotides in addition to those present in the sequence having at least 50% identity to SEQ NO: 2. Such additional oligonucleotides may be present at the 3′ end or the 5′ end of the antisense domain. Exemplary such antisense domains are highlighted in FIG. 23D and FIG. 23E, each of which show additional nucleotides (e.g., 5 nucleotides in addition to the 18-nt antisense domain used in the original construct) added to the 3′ end or the 5′ end of an antisense strand. In some embodiments, the antisense domain comprises a sequence as shown in Table 5 or Table 6.

In some embodiments, the antisense domain comprises a sequence shown in Table 5. In some embodiments, the antisense domain comprises a nucleotide sequence of SEQ ID NO: 202. In some embodiments, the antisense domain comprises a nucleotide sequence shown in Table 6. In some embodiments, the antisense domain comprises a nucleotide sequence of SEQ ID NO: 303. In some embodiments, the antisense domain comprises a nucleotide sequence of SEQ ID NO: 304.

In some embodiments, the guide RNA sequence comprises a recruitment domain. The recruitment domain (also referred to herein as the ADAR-recruiting part), facilitates the interaction with the ADAR or ADAR fusion protein. The recruitment domain is configured to bind (i.e., recruit) one or more ADAR proteins or fusions thereof. For example, the recruitment domain may be configured to recruit an ADAR1, or an ADAR2 protein or a fusion thereof. In some embodiments, the recruitment domain recruits at least an ADAR2 protein. The recruitment domain may comprise any suitable number of nucleotides. For example, the recruitment domain may comprise 15-100 nucleotides. In some embodiments, the recruitment domain comprises about 15, about 20, about 25, about 30, about 35, about 40, about 45, about 50, about 55, about 60, about 65, about 70, about 75, about 80, about 85, about 90, about 95, or about 100 nucleotides. In some embodiments, the recruitment domain is part of a construct that possesses a stem-loop secondary structure. In some embodiments, the recruitment domain forms a part of a stem-loop structure, wherein the loop portion of the stem loop structure consists of 5 nucleotides (i.e., a pentaloop).

In some embodiments, the recruitment domain comprises a first strand and a second strand that are substantially complementary or perfectly complementary to each other. In some embodiments, the first strand and the second strand are linked by a loop sequence. The loop structure may comprise any suitable number of nucleotides. In some embodiments, the loop structure comprises 3-50 nucleotides. In some embodiments, the loop structure comprises 3-50 nucleotides, 3-45 nucleotides, 3-40 nucleotides, 3-35 nucleotides, 3-30 nucleotides, 3-25 nucleotides, 3-20 nucleotides, 3-15 nucleotides, 3-10 nucleotides, or 3-7 nucleotides. In some embodiments, the loop structure is a pentaloop structure. Suitable sequences of a pentaloop structure are shown in Table 1. Any of the sequences shown in Table 1 may be used for a fusion construct as described herein. In some embodiments, the loop structure comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, OR SEQ ID NO: 18.

In some embodiments, the recruitment domain is based upon the sequence of an endogenous (i.e., naturally occurring) ADAR target. The recruitment domain may possess one or more modifications compared to the endogenous ADAR target, which may enhance ADAR recruitment or interactions. For example, the recruitment domain may be based upon the sequence of the GRIA2 R/G site, an endogenous target for ADAR2.

In some embodiments, the recruitment domain comprises a first strand (i.e., a 5′ strand) and a second strand (i.e., a 3′ strand) connected by a loop structure (also referred to herein as a loop sequence). The first strand and the second strand exhibit complementary base pairing, thus assisting in the formation of the stem loop structure of the construct. In some embodiments, this base pairing is disrupted by one or more mutations within the first strand and/or the second strand of the recruitment domain. In some embodiments, an unmodified recruitment domain refers to a recruitment domain that exhibits base pairing with no disruptions (i.e., perfect complementarity), whereas a mutated recruitment domain refers to a domain comprising one or more mutations in the first strand or the second strand that disrupt base pairing. In other words, an unmodified recruitment domain comprises a first strand with perfect complementarily to a second strand, whereas a mutated recruitment domain comprises a first strand and a second strand with substantial (i.e., at least 60%), but not perfect complementarity.

In some embodiments the recruitment domain comprises a first strand and a second strand connected by a pentaloop structure. In some embodiments, the first strand (i.e., the 5′ strand) comprises a nucleotide sequence haying at least 50% sequence identity to GGUGUCGAGAAGAGGAGAACAAUAU (SEQ ID NO: 3). For example, the first strand may comprise a nucleotide sequence having at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 3. In some embodiments, the first strand (i.e., the 5′ strand) comprises a sequence as shown in Table 2. In some embodiments, the first strand comprises a nucleotide sequence of SEQ ID NO: 108. In some embodiments, the first strand comprises a nucleotide sequence of SEQ ID NO: 109.

In some embodiments, the second strand comprises nucleotide sequence having at least 50% sequence identity to AUGUUGUUCUCGUCUCCUCGACACC (SEQ ID NO: 4). For example, the second strand may comprise a nucleotide sequence having at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4. In some embodiments, the second strand (i.e., 3′ strand) comprises a sequence as shown in Table 3. In some embodiments, the second strand comprises a nucleotide sequence of SEQ ID NO: 144. in some embodiments, the second strand comprises a nucleotide sequence of SEQ ID NO: 145. In some embodiments, the second strand comprises a nucleotide sequence of SEQ ID NO: 146.

In some embodiments, the first strand comprises a nucleotide sequence having at least 50% sequence identity to SEQ ID NO: 3 and the second strand comprises a nucleotide sequence having at least 50% sequence identity to SEQ ID NO: 4, and the first and second strand are connected by a loop structure. The loop structure may comprise any suitable number of nucleotides. In some embodiments, the loop structure comprises 3-50 nucleotides. In some embodiments, the loop structure comprises 3-50 nucleotides, 3-45 nucleotides, 3-40 nucleotides, 3-35 nucleotides, 3-30 nucleotides, 3-25 nucleotides, 3-20 nucleotides, 3-15 nucleotides, 3-10 nucleotides, or 3-7 nucleotides. In some embodiments, the loop structure is a pentaloop (i.e., comprises 5 nucleotides). In some embodiments, the loop structure comprises a sequence set forth in Table 1. In some embodiments, the loop structure comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, OR SEQ ID NO: 18.

In some embodiments, the guide RNA comprises a combination of mutations. In some embodiments, the guide RNA comprises at least 2 mutations (i.e., 2, 3, 4, 5, or more than 5) mutations. For example, the guide RNA may comprise one or more mutations within the antisense domain (i.e., one or more mutations that disrupt a given base pairing with a corresponding nucleotide in the target sequence) and one or more mutations within the recruitment domain of the guide RNA (i.e., one or more mutations that disrupt or restore base pairing between the first strand and the second strand of the recruitment domain). In some embodiments, the guide RNA comprises multiple mutations in the recruitment domain. In some embodiments the guide RNA comprises an antisense domain as set forth in Table 4, Table 5, or Table 6, and a loop sequence set forth in Table 1. In some embodiments, the guide RNA comprises an antisense domain as set forth in Table 4, Table 5, or Table 6, and a recruitment domain comprising a first sequence as set forth in Table 2 and/or a second sequence as set forth in Table 3. In some embodiments, the construct comprises an antisense domain as set forth in Table 4, Table 5, or Table 6, a loop sequence as set forth in Table 1, and recruitment domain comprising a first sequence as set forth in Table 2 and/or a second sequence as set forth in Table 3.

The guide RNAs described herein find use in methods of site directed RNA editing (e.g., site directed A-to-I RNA editing) in a cell or a subject. For example, RNA editing may be performed to treat a disease or condition in a subject. For example, the guide RN As described herein may be used in methods of treating diseases or conditions characterized by G to A point mutations in a gene expressed by the subject. In some embodiments, the disease is Hurler Syndrome.

In some embodiments, the guide RNA or construct comprising the same may be formulated into a composition for delivery to the cell or subject. For example, the construct may be formulated into a composition for parenteral administration. The term “parenteral” refers to any suitable non-oral route of administration, including subcutaneous, intramuscular, intravenous, intrathecal, intracisternal, intraarterial, intraspinal, intraepidural, intradermal, and the like. The construct may be formulated with any suitable excipients, stabilizers, preservatives, and the like. In some embodiments, the composition may be provided to a subject suffering from Hurler Syndrome. Accordingly, in some embodiments provided herein are methods for treating Hurler Syndrome, comprising providing to a subject in need thereof a composition comprising a gRNA as described herein (i.e., an optimized gRNA). The gRNA may he identified using a high-throughput screening method as described herein.

It is understood that endogenous ADARs and/or engineered ADAR fusions may be suitable for use in the methods for site-directed RNA editing described herein. For example, the guide RNAs (including optimized guide RNAs) identified by a screening method described herein may be well suited for use with ADAR fusion proteins in the methods described herein.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1—Optimizing the gRNA Sequence

Overview on the screening platform: Efficient editing generally depends on many factors, such as substrate sequence and the length and structure of the gRNA/target duplex.^{48,49} The present knowledge does not allow conclusions how to design a gRNA which enables ADAR enzymes to edit a certain site with the highest possible efficiency. To overcome this hurdle, next-generation sequencing (NGS) may be used to screen gRNA library sequences for their ability to edit the G-to-A point mutations. In a real scenario, editing is performed in a target transcript when it is bound by the gRNA which is able to recruit the ADAR, enzyme. For the NGS-based screen, the target sequence and the ASO sequence are expressed in the same transcript, such that they may be identified on a single sequencing read to know which editing level is mediated by which ASO sequence. In order to achieve this, target regions, containing the pathogenic G-to-A point mutations, may be obtained from the full-length transcripts and fused to the ASO library sequences, resulting in hairpin structures which simulate the duplex between the target RNA and a transacting gRNA. The design of the target RNA/gRNA libraries is described in more detail in Example 2.

For screening experiments, target RNA/gRNA fusion libraries may be ordered as DNA oligonucleotides and ligated into an expression vector. For example, libraries may be ligated into an expression vector using a well-established clone-and-use strategy.^{50,51} The resulting plasmid libraries may be delivered to human ADAR-expressing cells by a suitable method, such as via lipofection. After the incubation with the plasmid library, RNA may be isolated from the cells and cDNA of the target RNA/gRNA fusions may be prepared for their subsequent sequencing with NGS (Illumina sequencing). For the sequencing library preparation, NGS adapters with different indexes may be used, which allows the concurrent analysis of multiple experiments. To analyze the sequencing data, a computational pipeline may be used which enables the detection of editing levels within the target RNA sequences and the identification of the corresponding gRNAs. Alternatively, target/gRNA fusions may be in-vitro transcribed and transfected into the cells without the need of a plasmid.

The comparison between the induced editing levels at the target site reveals which gRNA sequences can direct ADARs for efficient RNA editing. Additionally, examining the extent of editing of off-site adenosines within the target RNA/gRNA fusions shows how precisely the gRNAs mediate RNA editing. The impact of the target RNA/gRNA duplex structure and sequence on the editing efficiency and specificity may be also evaluated by the analysis.

### Example 2

### Design of Target RNA/gRNA Fusion Libraries

gRNAs that enable ADARs to catalyze site-directed RNA editing comprise two parts: an antisense domain for binding to the target sequence, and an imperfect double-stranded ADAR-recruiting part, which ensures the interaction with the ADAR enzyme (FIG. 2).

Since RNA editing can be influenced by multiple factors, it appears likely that maximum editing requires a tailored gRNA sequence for each site. In order to find those optimal gRNA sequences, screening the gRNA antisense and ADAR-recruiting part with every target of interest may be performed.

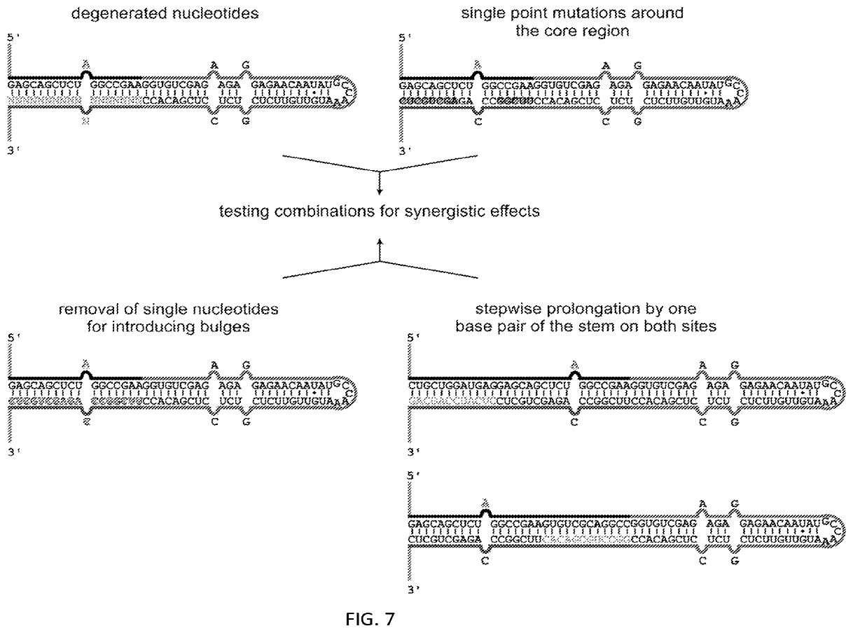

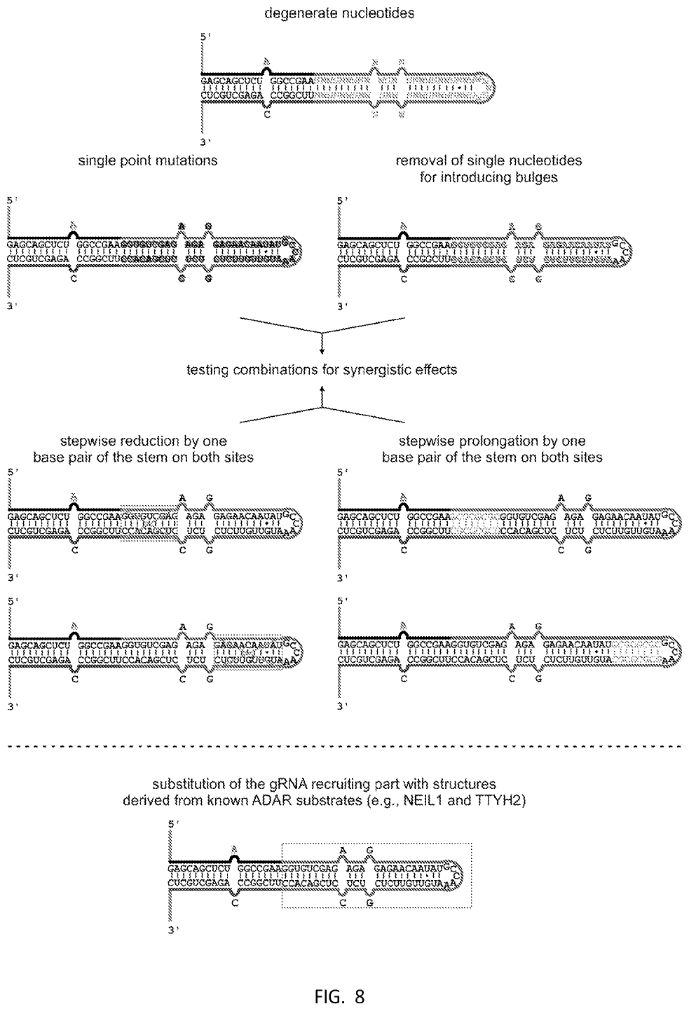

Target RNA/gRNA libraries for the identification gRNA sequences that maximize RNA editing may be designed. Single point mutations or a stretch of degenerate nucleotides may be introduced in both gRNA parts (antisense and recruitment domains), leading to mismatches, Watson-Crick base pairs or wobble base pairs in the target RNA/gRNA duplex structure and in the recruitment domain (FIG. 7, FIG. 8).

The methods described herein may be used to identify mismatches at certain positions, which increase the editing level at the target site. Additionally, single nucleotides may be removed (or inserted) to introduce bulges which might also improve the editing yield. Stepwise reduction (ADAR-recruiting part) or prolongation (antisense and ADAR-recruiting part) of the RNA stems may also be tested (FIG. 7, FIG. 8).

Furthermore, other ADAR-recruiting parts derived from known editing substrates (FIG. 8) may be used for improved editing capabilities. Multiple features that are found to enhance editing are combined as desired.

The optimized gRNA sequences identified by the methods described herein may be combined in a modular fashion with other guide designs known to enhance the efficiency and/or specificity of editing. For example, mismatches in the antisense region that are shown to enhance editing in the screen may be incorporated into circular guides or into guides consisting of a long antisense domain without a recruitment domain.

### REFERENCES

1 Jinek, M. et al. A Programmable Dual-RNA—Guided DNA Endonuclease in Adaptive Bacterial Immunity. Science 337 816 (2012).
2 Komor, A. C., Badran, A. H. & Liu, D. R. CRISPR-Based Technologies for the Manipulation of Eukaryotic Genomes. Cell 168, 20-36 (2017).
3 Haapaniemi, E., Botla, S., Persson, J., Schmierer, B. & Taipale, J. CRISPR-Cas9 genome editing induces a p53-mediated DNA damage response. Nat. Med. 24, 927-930 (2018).
4 Ihry, R. J. et al. p53 inhibits CRISPR-Cas9 engineering in human pluripotent stem cells. Nat. Med. 24, 939-946 (2018).
5 Wagner, D. L. et al. High prevalence of Streptococcus pyogenes Cas9-reactive T cells within the adult human population. Nat. Med. 25, 242-248 (2019).
6 Simhadri, V. L. et al. Prevalence of Pre-existing Antibodies to CRISPR-Associated Nuclease Cas9 in the USA Population. Molecular therapy. Methods & clinical development 10, 105-112 (2018).
7 Charlesworth, C. T. et al. Identification of preexisting adaptive immunity to Cas9 proteins in humans. Nat. Med., doi:10.1038/s41591-018-0326-x (2019).
8 Vogel, P. & Stafforst, T. Critical review on engineering deaminases for site-directed RNA editing. Curr. Opin. Biotechnol. 55, 74-80 (2019).
9 Montiel-Gonzalez, M. F., Diaz Quiroz, J. F. & Rosenthal, J. J. C. Current strategies for Site-Directed RNA Editing using ADARs. Methods, doi:10.1016/j.ymeth.2018.11.016 (2018).
10 Picardi, E. et al. Profiling RNA editing in human tissues: towards the inosinome Atlas. Sci. Rep. 5, 14941 (2015).
11 Bazak, L. et al. A-to-I RNA editing occurs at over a hundred million genomic sites, located in a majority of human genes. Genome Res. 24, 365-376 (2014).
12 Tan, M. H. et al. Dynamic landscape and regulation of RNA editing in mammals. Nature 550, 249-254 (2017).
13 Nishikura, A-to-I editing of coding and non-coding RNAs by ADARs Nat. Rev. Mol. Cell Biol. 17, 83-96 (2016).
14 Walkley, C. R. & Li, J. B. Rewriting the transcriptome: adenosine-to-inosine RNA editing by ADARs. Genome Biol. 18, 205 (2017).
15 Azad, M. T. A., Bhakta, S. & Tsukahara, T. Site-directed RNA editing by adenosine deaminase acting on RNA for correction of the genetic code in gene therapy. Gene Ther. 24, 779 (2017).
16 Katrekar, D. et al. in vivo RNA editing of point mutations via RNA-guided adenosine deaminases. Nat. Methods, doi.10.1038/s41592-019-03234) (2019).
17 Cox, D. B. T. et al. RNA editing with CRISPR-Cas13. Science 358, 1019-1027 (2017).
18 Montiel-Gonzalez, M. F., Vallecillo-Viejo, I., Yudowski, G. A. & Rosenthal J. J. C. Correction of mutations within the cystic fibrosis transmembrane conductance regulator by site-directed RNA editing. Proc. Natl. Acad. Sci. USA 110, 18285-18290 (2013).
19 Montiel-González, M. F., Vallecillo-Viejo, I. C. & Rosenthal, Joshua J. C. An efficient system for selectively altering genetic information within mRNAs. Nucleic Acids Res. 44, e157-e157 (2016).
20 Sinnamon, J. R. et al. Site-directed RNA repair of endogenous Mecp2 RNA in neurons. Proc. Natl. Acad. Sci. USA 114, E9395-E9402 (2017).
21 Stafforst, T. & Schneider, M. F. An RNA-deaminase conjugate selectively repairs point mutations. Angew. Chem. Int. Ed. 51, 11166-11169 (2012).
22 Vogel, P., Schneider, M. F., Wettengel, J. E Stafforst, T. Improving Site-Directed RNA Editing in Vitro and in Cell Culture by Chemical Modification of the GuideRNA. Angew. Chem. Int. Ed. 53, 6267-6271 (2014).
23 Vogel, P. et al. Efficient and precise editing of endogenous transcripts with SNAPtagged ADARs. Nat. Methods 15, 535-538 (2018).
24 Keppler, A. et al. A general method for the covalent labeling of fusion proteins with small molecules in vivo. Nat. Biotech. 21, 86-89 (2003).
25 Hanswillemenke, A., Kuzdere, T., Vogel, P., Jékely, G. & Stafforst, T. Site-Directed RNA Editing in Vivo Can Be Triggered by the Light-Driven Assembly of an Artificial Riboprotein. J. Am. Chem. Soc. 137, 15875-15881 (2015).
26 Vogel, P., Hanswillemenke, A. & Stafforst, T. Switching Protein Localization by SiteDirected RNA Editing under Control of Light. ACS Synth. Biol. 6, 1642-1649 (2017).
27 Vallecillo-Viejo, I. C., Liscovitch-Brauer, N. Montiel-Gonzalez, M. F., Eisenberg, E. Rosenthal, J. J. C. Abundant off-target edits from site-directed RNA editing can be reduced by nuclear localization of the editing enzyme. RNA Biol. 15, 104-114 (2018).
28 Wettengel, J., Reautschnig, P., Geisler, S., Kahle, P. J. & Stafforst, T. Harnessing human ADAR2 for RNA repair—Recoding a PINK1 mutation rescues mitophagy. Nucleic Acids Res. 45, 2797-2808 (2017).
29 Fukuda, M. et al. Construction of a guide-RNA for site-directed RNA mutagenesis utilising intracellular A-to-I RNA editing. Sci. Rep. 7, 41478 (2017).
30 Heep, M., Mach, P., Reautschnig, P., Wettengel, J. Stafforst, T. Applying Human ADAR1 p110 and ADAR1 p150 for Site-Directed RNA Editing—G/C Substitution Stabilizes GuideRNAs against Editing. Genes 8, 34 (2017).
32 Merkle, T. et al. Precise RNA editing by recruiting endogenous ADARs with antisense oligonucleotides. Nat. Biotechnol. 37, 133-138 (2019).
33 Miklossy, G., Hilliard, T. S. & Turkson, J. Therapeutic modulators of STAT signalling for human diseases, Nat. Rev. Drug Discov. 12, 611 (2013).
46 Kawahara, Y. et al. Glutamate receptors: RNA editing and death of motor neurons. Nature 427, 801-801 (2004).
47 Bennett, C. F., Baker, B. F., Pham, N., Swayze, E. & Geary, R. S. Pharmacology of Antisense Drugs. Annu. Rev. Pharmacol. Toxicol. 57, 81-105 (2017).
48 Eggington, J. M., Greene, T. & Bass, B. L. Predicting sites of ADAR editing in doublestranded RNA. Nat. Commun. 2, 319 (2011).
49 Wong, S. K., Sato, S. & Lazinski, D. W. Substrate recognition by ADAR1 and ADAR2. RNA 7, 846-858 (2001).
50 Bassik, M. C. et al. Rapid creation and quantitative monitoring of high coverage shRNA libraries. Nat. Methods 6, 443-445 (2009).
51 Shalom, O. et al. Genome-scale CRISPR-Cas9 knockout screening in human cells. Science 343, 84-87 (2014).
70 Jing X et al. Implementation of the CRISPR-Cas13a system in fission yeast and its repurposing for precise RNA editing. Nucleic Acids Res (2018)

### Example 3

### Screening Methods

Designing and testing the ASO library prototype: The ASO library prototype was based on the published ASO design ‘v9.4’³², with the key distinction that an 18-nucleotide (nt) region of the target sequence was included as part of a fusion construct mimicking the guide/target complex (FIG. 9A). This fusion construct uniquely enables capturing the guide RNA sequence and the associated editing events in the same sequencing read. Additionally, the hairpin loop sequence in the recruitment domain was changed from ‘GCUAA’ to ‘GCCAA’ to eliminate a stop codon.

The target sequence probed in the pilot screen comprised an 18-nt region from the human IDUA gene, containing the G-to-A mutation observed in Hider syndrome patients, flanked by 10 upstream and 7 downstream residues from the wild-type IDUA sequence. The guide RNA portion of the fusion construct comprised a recruitment domain, followed by an 18-nt antisense sequence. The recruitment domain was based on ADAR's endogenous GRIA2 R/G site, and included several sequence substitutions to suppress editing within the recruitment domain³². The antisense sequence was complementary to the target sequence, except for a C mismatch opposite from the editing site, which was previously found to increase editing⁴⁹.

Prior to screening, it was important to ensure that the library prototype was edited detectably, but not to completion under the screening conditions, to provide sufficient dynamic range for identifying enhancer variants. Thus, the editing of the prototype was first tested in Flp-In T-REx 293 cells with and without inducible ADAR1 p150 expression. The prototype was restriction-cloned into a pcDNA5 vector, as a spacer region between mCherry and EGFP coding sequences (see details in the Cloning section). Flp-In T-REx 293 cells with integrated ADAR1 p150 were seeded in a 24-well tissue culture plate (350,000 cells/well), in the presence or absence of 10 ng/ml doxycycline (Dox). After 20 h, 500 ng plasmid was transfected with 2.5 μL Lipofectamine 2000 by pipetting dropwise. 24 h later, the total RNA was isolated and purified using an RNeasy MinElute kit (Qiagen) and reverse-transcribed with an mCherry-specific primer using the M-MuLV reverse transcriptase (NEB). The PCR-amplified, agarose gel-purified cDNA was Sanger-sequenced to determine the editing level. The observed editing was ˜50% in the presence of endogenous ADAR only (no Dox induction), and 100% with Dox induction (FIG. 9B, C). Thus, FlpIn T-REx cells expressing only endogenous ADAR protein were used for subsequent screening.

To obtain appropriate baseline editing level for other prototypes (i.e., detectable, but <<100%), many variables can be manipulated, including prototype design, cell type, doxycycline concentration, knock out of endogenous ADAR proteins, or time. Several variations of the guide/target fusion have been tested. For example, the recruitment domain can be omitted, instead using longer target and antisense sequences that are connected by a short loop (FIG. 9D, E). This design allows to probe the target-specific sequence features that affect editing across a longer region without creating excessively stable RNA structures that may interfere with the screening protocol. In an extension of this design, the target and guide sequences are separated by the EGFP coding sequence (720 nt+short linkers) instead of a short loop (FIG. 9F). This design, in which the target and guide sequences are spatially separated by a translated sequence more closely mimics editing with trans guides.

To expedite identification of one or more prototypes for a new target, to be used as reference sequence for subsequent high-throughput library design, a small initial screen may be performed by using an oligonucleotide pool containing different prototype designs. Such a pool of 10 s or 100 s of designs could include systematic variation of the following parameters: length of the target and antisense regions; position of the editing site within the construct; identity of the recruitment domain (if present). The oligonucleotide pool could be obtained, e.g., as an IDT oPool or a small Twist/Agilent oligonucleotide library. The oligos could be cloned and screened analogous to the full-scale screening procedure below, scaled down appropriately.

Library design—To obtain a library of antisense variants targeting the IDUA W402X mutation, the antisense region in FIG. 9A was randomized, such that at each position the ‘consensus’ base, displayed in the prototype, was present 82% of the time, and each of the other 3 bases was present 6% of the time. This degeneracy level was chosen to give complete representation of single and double mutants of the antisense region in a ˜10,000 variant library, while still sampling a substantial number of higher-order mutants. This degeneracy level should be adjusted depending on the length of the randomized sequence, the size of the desired library, and the desired mutant coverage. Randomized residues can be introduced anywhere in the guide sequence, spanning the entire guide sequence or, e.g., only including residues near the editing site, and the number of randomized residues can be varied.

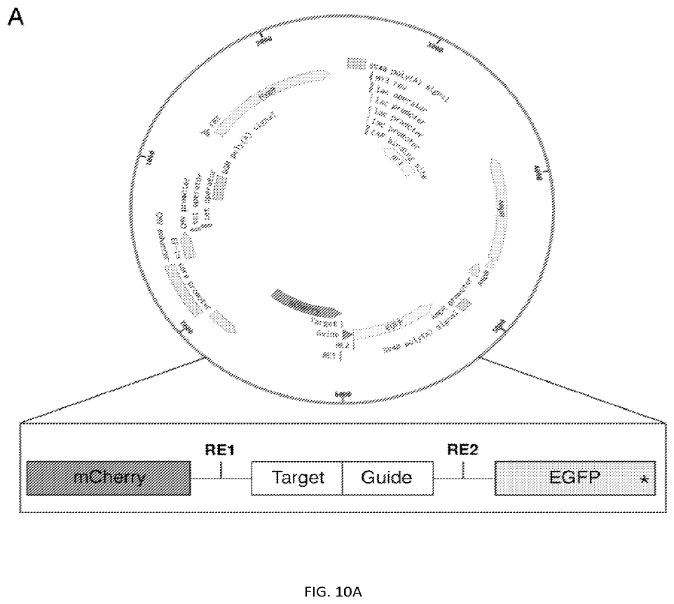

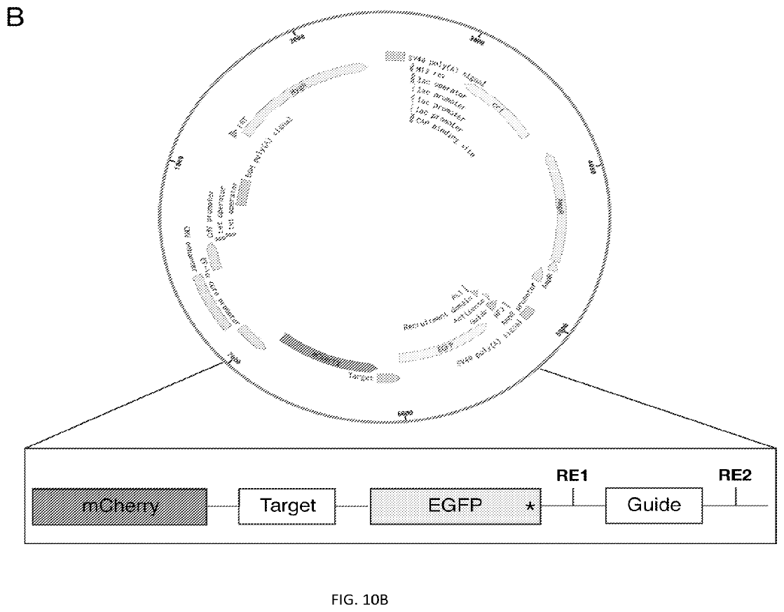

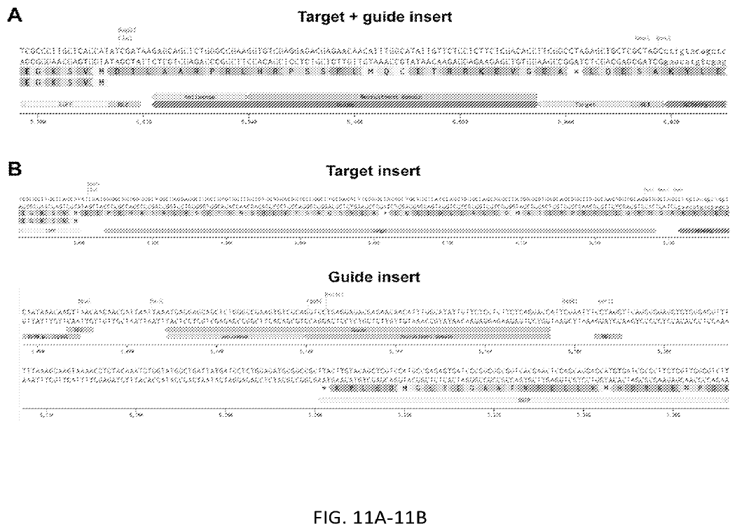

Cloning—The ASO library based on the prototype in FIG. 9 was cloned into a pcDNA5 vector between the mCherry and EGFP coding sequences (FIG. 10). To mimic editing within a translated region (as most therapeutic editing will likely target coding sequences), the mCherry stop codon was removed upstream from the target sequence. Alternative vectors, in which the guide-target fusion is expressed within the 3′UTR of an EGFP mRNA or as an RNA polymerase III-transcribed small-RNA library, may also be used. FIG. 10 shows exemplary vectors and arrangements that may be used, but they are not to be construed as limiting in any way. The vectors used for cloning are not limited to any particular order or arrangement of coding sequences (e.g., mCherry, EGFP, target RNAs, or guide RNAs).

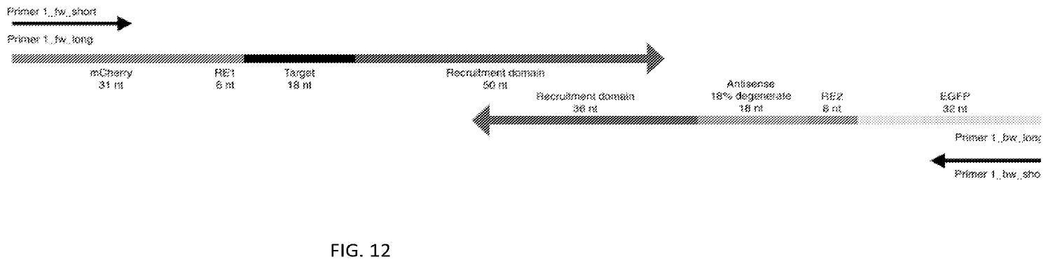

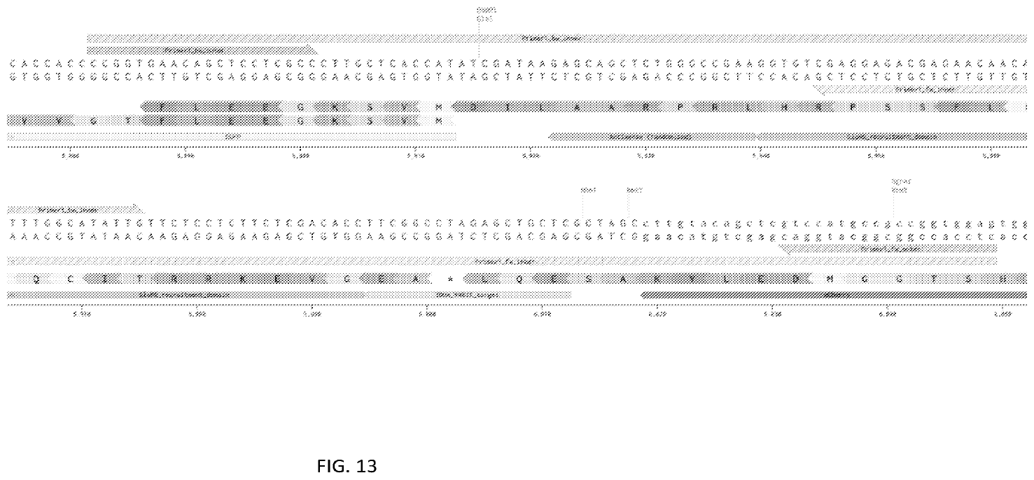

Prior to cloning, the ASO library insert was PCR-assembled from two single-stranded DNA oligonucleotides, partially overlapping in the recruitment domain and containing either the target or the randomized antisense region (FIG. 12, FIG. 13). The primer containing the randomized region (‘Primer1_bw_inner’ in FIG. 12, FIG. 13) was produced by Stanford's PAN facility, using brand-mixed bases to obtain 18% degeneracy. The primers are also available commercially, such as from IDT. All other oligonucleotides mentioned below were obtained from IDT. The PCR assembly was performed with 1.5 nM of the long primers and 500 nM of the short terminal primers using the KOD Xtreme™ Hot Start DNA polymerase (Novagen.). The annealing temperature was 62° C. (30 s), and the extension step was performed for 15 s at 68° C. The library was amplified for 16 cycles, corresponding to half-saturation, as determined by quantitative real-time PCR. (qRT-PCR). The KOD Xtreme polymerase is optimized for highly structured templates and is therefore strongly recommended for library preparation. Alternatively, double-stranded (ds) DNA fragments encompassing the full ASO fusion construct and flanking regions, with a limited number of randomized positions, can be commercially obtained, e.g., from IDT.

To prevent PCR byproducts and to eliminate the need for gel purification, here and below, all PCR reactions were performed for a number of cycles corresponding to half-saturation, as determined by qRT-PCR. The purity of all PCR products was evaluated by polyacrylamide gel electrophoresis (PAGE, Novex 6% acrylamide gels with TBE; Invitrogen; post-stained with 1× SYBR-Gold).

The dsDNA product was purified with the Macherey-Nagel PCR purification kit and restriction-cloned into the pcDNA5 vector between mCherry and EGFP coding sequences, using ClaI and NheI restriction enzymes and T4 DNA ligase. The ligation reaction was performed using a 5-fold molar excess of insert over vector, as determined with NEBioCalculator. After a 30 min incubation at room temperature and a 3-h incubation at 16° C., the reaction was heat-inactivated for 10 min at 65° C., and the DNA was purified and concentrated using the Macherey-Nagel PCR purification kit. To obtain a ˜10,000 variant library, 50 ng of DNA (in a 2 μL volume) was transformed into 25 μL of TOP10 competent cells (Invitrogen). The cells were plated on two 15-cm LB-Carb 100 plates (Teknova) and incubated overnight at 37° C. To obtain larger libraries, the amount of ligated DNA, the cell volume, and the number of plates should be increased proportionally.

Approximately 10,000 colonies were harvested from LB-Carb plates by gently scraping the plate with a razor blade and washing with LB-broth. The plasmid DNA was purified on a HiSpeed Plasmid Midi column (Qiagen).

To achieve higher-throughput, on the scale of 100,000 colonies, electrocompetent cells (such as Lucigen Endura) should be used, and the cells can be plated on 245 mm×245 mm LB-Carb plates. The plasmid DNA should be isolated using a Maxi prep (e.g., HiSpeed Plasmid Maxi kit from Qiagen).

Cell culture—Flp-In T-REx 293 cells with an integrated empty pcDNA5 vector were maintained in DMEM medium (Gibeo), supplemented with 10% FBS, 100 μg/ml Hygromycin B, 15 μg/ml blasticidin, and 100 U/ml Gibco™ Penicillin-Streptomycin. It was found that inducible ADAR1 expression in Flp-In T-REx cells with integrated ADAR1 p150 was unnecessary to observe sufficient editing levels (FIG. 9B, C); consequently, Flp-In T-REx 293 cells containing an empty pcDNA5 vector and thus expressing only endogenous ADAR proteins were used for screening. Use of Flp-In T-REx cells is not required for the screening protocol, and any other cell lines that express sufficient ADAR protein for detectable editing and are amenable to transfection can be used for screening.

Screening procedure—One and a half million 293 Flp-In T-REx cells with an integrated empty pcDNA5 vector were seeded per well of a 6-well tissue-culture coated plate and incubated at 37° C. Twenty two hours later (corresponding to ˜70% cell confluency), the plasmid library (2.75 μg) and Lipofectamine 2000 (8.25 μL) were separately diluted in OptiMEM (550 μL final volume) and were incubated at room temperature for 5 min. The two solutions were mixed and incubated for 20 min, and 1 ml of the mix was added dropwise to the plated cells, 24 h later, the media was removed, and the cells were harvested by pipetting up and down. The screening results were unaffected by changing the transfection scale to 10 μg DNA, transfected into 5 million cells seeded on a 10 cm plate. The time between library transfection and harvesting the cells also did not affect screening outcomes, when varied between 7 h and 48.5 h.

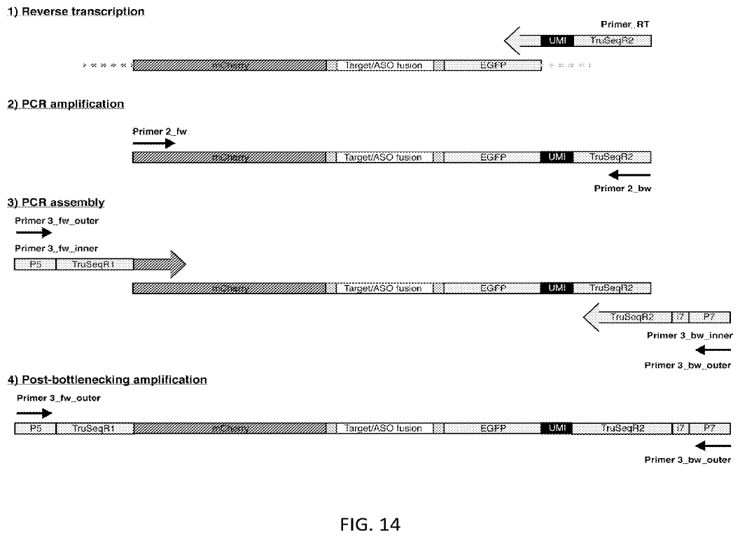

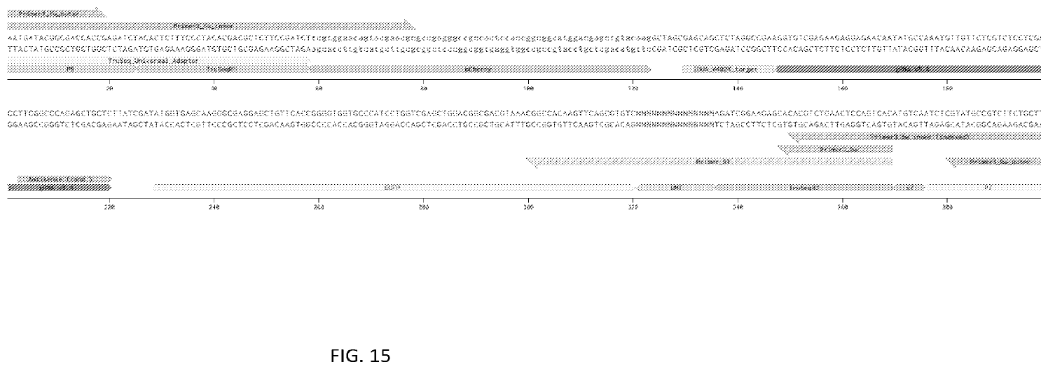

Total RNA was purified on a single RNeasy Mini column (Qiagen). For larger-scale transfections, multiple RNeasy Mini columns or an RNeasy Midi column may be required, as determined by the cell type and number, and the column capacity stated in the manual. Total RNA (150 ng/μL) was treated with Turbo DNase (Invitrogen) for 30 min at 37° C., and the reaction was stopped with 1/10th volume of DNase Inactivating reagent (Invitrogen), following the manufacturer's protocol. Reverse transcription (RT) was performed with the TGIRT III enzyme (InGex), which is optimized for highly structured RNA templates. Comparable performance was achieved with the WarmStart RTx Reverse Transcriptase (NEB). Other reverse transcriptases may lead to the loss of library variants with the most stable secondary structures and distorted editing measurements due to truncated reverse transcription products. The TGIRT reaction (20 μL) included 9.7 μL of Turbo DNase-treated total RNA, 10 mM dithiothreitol (DTT), 0.1 μM barcoded RT primer (FIG. 14, FIG. 15), 1× TGIRT buffer, 1 μL TGIRT enzyme, and 1.25 mM dNTPs (added after 30 min pre-incubation of the other components at room temperature). The no-RT control was prepared identically except for 1 μL water being used instead of the TGIRT enzyme. Both the RT and no-RT reactions were incubated at 60° C. for 1 h. After cooling to room temperature, 1 μL of 5 M NaOH was added, followed by incubation at 95° C. for 3 minutes. After cooling to room temperature, the reaction was neutralized with 2.5 μL of 2 M HCl, and the volume was adjusted to 50 μL with water, followed by purification with the Macherey-Nagel PCR purification kit. Including the no-RT control is essential for ensuring that plasmid DNA has been effectively removed by the DNase treatment and for the detection and troubleshooting of possible primer byproducts in the subsequent PCR step. The purified cDNA and identically treated no-RT control were amplified using the KOD Xtreme DNA polymerase, which was also used for all subsequent PCR steps (FIG. 14). 0.3 μM each of Primer_2_fw and Primer_2_bw and 1/10th volume of purified RT or no-RT product was used for the PCR reaction, with an annealing temperature of 57° C. and an extension step of 20 s at 68° C. The number of PCR cycles was determined by qRT-PCR (corresponding to ˜50-75% of saturating signal), and the purity of the DNA product was confirmed by 6% PAGE. The efficiency of plasmid DNA removal was confirmed by comparing the Cₜ values (as determined by qRT-PCR) between the PCR reactions using the RT and no-RT reaction as template. A Cₜ difference of at least ˜7 is desired, corresponding to at least a ˜100-fold difference in abundance of cDNA and plasmid DNA. Additionally, the PCR products of the RT and no-RT reactions were compared on a gel, by running both PCR reactions for the same number of cycles, corresponding to mid-saturation of the reaction with the RT template (as determined by qRT-PCR); then analyzing aliquots of both PCR reactions by 6% PAGE. The no-RT reaction should give no detectable signal. The PCR-amplified cDNA library was purified using the Macherey-Nagel PCR purification kit and the DNA concentration was determined with Qubit. Illumina sequencing adapters were subsequently added through PCR assembly, as shown in FIG. 14, by including 0.5 nM template, 1.5 nM of each of the long inner primers (‘Primer3_fw_inner’ and ‘Primer3_bw_inner’) and 0.3 μM each of the short outer primers (‘Primer3_fw_outer’ and ‘Primer3_bw_outer’). The annealing temperature was 55° C. and the extension step was performed for 30 s at 68° C. Primer3_bw_inner contained a 6-nt i7 index, and a different i7 index was used for every unique library to enable pooled sequencing. The purity of the assembled product was confirmed by 6% PAGE, and the library was purified with the Macherey-Nagel PCR purification kit.

The RT primer contains a unique molecular identifier (UMI), which is essential for accurate quantification of editing levels (FIG. 14, FIG. 15). To ensure that each UMI, signifying a unique cDNA, is represented by multiple reads during subsequent sequencing, the library was bottlenecked, such that each library variant was represented by, on average, 100 UMIs. To achieve this, the concentration of the assembled cDNA was measured by Qubit, and the sample was serially diluted until it contained 1,000,000 (=100 UMIs×10,000 variants) molecules per μL. One μL of the diluted sample was then used as template in the bottlenecking PCR reaction (FIG. 14; annealing temperature of 57° C., extension for 30 s at 68° C.), and the reaction was purified with the Macherey-Nagel PCR purification kit^{71, 72}. To avoid loss of DNA due to sticking to the tubes and pipette tips at the low DNA concentrations used during the bottlenecking step, serial dilutions were performed in a 100 nM solution (in 0.1% Tween 20) of primers used for the subsequent PCR amplification instead of water/TE buffer (‘Primer_3_fw_outer’ and ‘Primer_3_bw_outer’ in FIG. 14, FIG. 15). Bottlenecking to an average of 100 UMIs, corresponding to 100 unique cDNAs, per variant allows for accurate quantification of edited and unedited RNAs associated with the same antisense variant. The library was sequenced using HiSeq (Illlumina) with paired-end 150 by reads. The IDUA W402X library was multiplexed with other individually indexed libraries in a single HiSeq lane, with an average of 20 reads allotted per UMI. Alternatively, an Illumina MiSeq kit can be used for sequencing a single 10,000 variant library. In contrast to HiSeq and MiSeq, we found that Illumina NextSeq and NovaSeq platforms yielded insufficient sequencing quality in the hairpin region of the library constructs, preventing reliable sequence identification and quantification of editing levels. Consequently, NextSeq and NovaSeq should not be used for screening.

To improve the sequencing quality, sequence diversity was increased by mixing the cDNA library with about 40% of PhiX Sequencing Control V3 (Illumina). To rigorously distinguish between real editing events and unintended A-to-G mutations at the DNA level, the plasmid DNA library was also sequenced. The DNA library was prepared for sequencing by using the same primers as those used for cDNA library preparation, starting with the ‘PCR amplification’ step (FIG. 14). At this step, 0.2 ng/μL plasmid library was amplified using 0.3 μM Primer2_fw and Primer2_fw, as well as 1.5 nM of a truncated version of the barcoded Primer_RT (FIG. 14), shortened by 2 nt at the 3′ end to match the melting temperature of Primer2_fw and Primer2_bw (57° C.), which was different from the optimal RT temperature (60° C.). The following steps were identical to those in cDNA library preparation, including the bottlenecking step. An exemplary construct and primers for cDNA and DNA library preparation are shown in FIG. 15.

Analysis-Paired-end reads were merged using FLASH-1.2.11, truncated reads were removed, and the UMI sequence, as well as the library variant sequence in each read were identified based on their positions relative to the constant mCherry and EGFP sequence regions. Reads containing non-redundant UMIs (i.e., UMIs present in a single read) were removed from further analysis. The remaining reads were grouped by their respective UMI sequence, and a consensus sequence of the target-guide fusion was determined, based on the sequence observed in two or more reads containing the same UMI. Alternatively, more stringent criteria could be used for consensus determination, requiring, e.g., that at least half of the reads feature the same variable sequence (Buenrostro et al., 2014). If all reads containing a given UMI had distinct sequences in the target-guide fusion region, no consensus was available and the corresponding reads were discarded. Since errors are unlikely to simultaneously occur both in the UMI and in the variable guide RNA region, this consensus-based procedure allows to reliably identify library variants and edited residues even in the presence of sequencing or PCR errors. These and subsequent analyses were performed using custom Python scripts.

After identifying the UMI consensus, editing levels associated with each guide RNA variant were quantified as follows. Sequences with non-A-to-G changes in the target sequence or in the recruitment domain were removed from further analysis. Only guide RNA variants (including variants of the antisense or recruitment domain regions) that were represented by at least 10 UMIs were propagated to further analysis to ensure accurate quantification. For each guide RNA sequence, UMIs were counted for each of the following versions of the target sequence: (1) intact target sequence (‘Unedited’); (2) target sequence with A-to-G change at the intended site, regardless of any additional off-target editing (‘Edited’); (3) target sequence featuring only unintended A-to-G changes, without on-target editing (‘Off-target’).The fraction of variants edited at the intended site was calculated as follows:

$$Fraction edited = \frac{\# {UMI}_{Edited}}{\# {UMI}_{Edited} + \# {UMI}_{Unedited} + \# {UMI}_{Off - target}}$$

By counting UMIs (which signify unique cDNAs), rather than analyzing raw sequencing reads, this quantification method reduces the effects of potentially uneven sequence representation, arising from PCR bias or other technical artifacts.

While off-target editing was rare in the case of IDUA, it may he more prevalent for A-rich. target sequences (or recruitment domains). In these cases, detailed analysis of variants with unintended editing events should be performed, as it can inform efforts to design more specific guides and strategic positioning of chemical modifications.

To account for spurious editing events arising from A to-G mutations at the DNA level (within the target sequence or guide RNA), the cDNA library was cross-referenced against the parallelly sequenced plasmid DNA library. A-to-G mutation rates observed in the DNA library were subtracted from the corresponding editing levels for each antisense variant. Sequencing the DNA library may also allow distinguishing between real antisense variants featuring G mutations and rare A-to-G editing events in the antisense region, as the relative representation of such variants would differ between cDNA and DNA libraries.

Exemplary guide RNA variants (i.e., ASOs) that may be selected and/or optimized by a platform described herein, such as the methods described in Example 3, are shown in the following figures and tables.

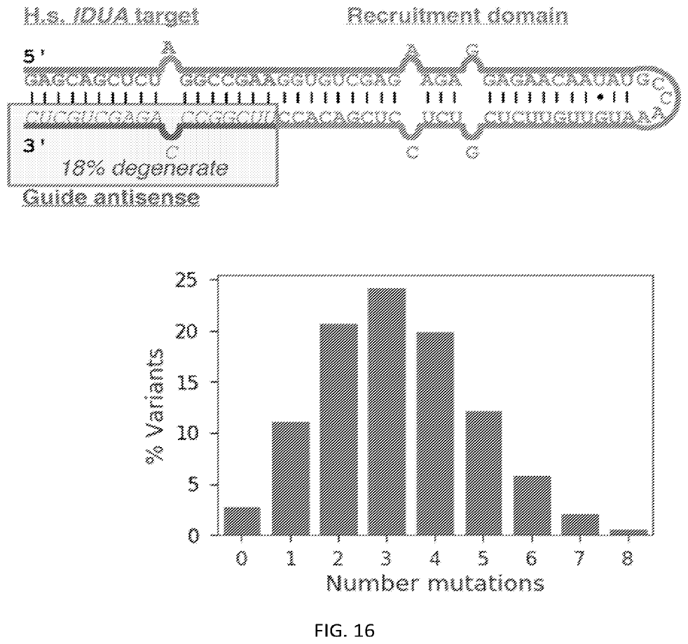

FIG. 16 shows an exemplary hairpin construct (comprising a recruitment domain, a target sequence, and a guide antisense oligonucleotide) targeting IDUA W402X, which may be generated by methods described herein in particular as described in Example 3.

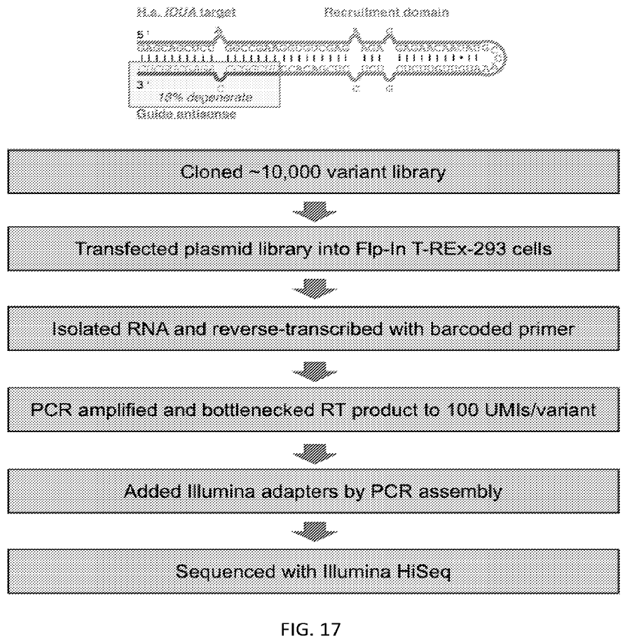

FIG. 17 shows an exemplary workflow, as described herein and in particular in Example 3.

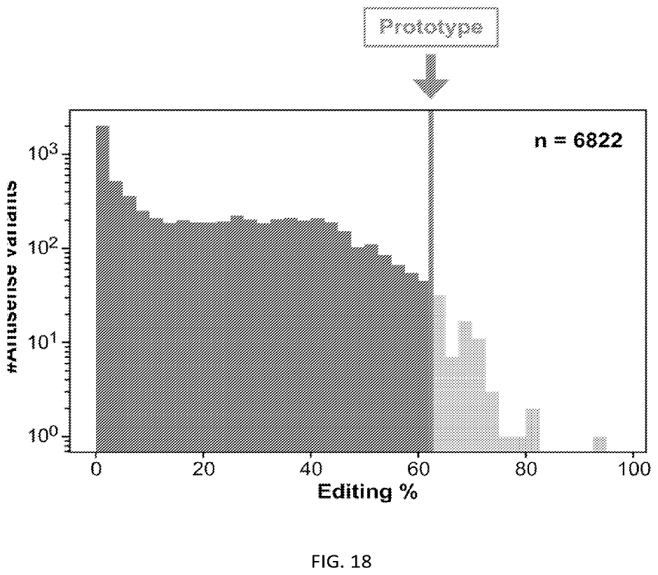

FIG. 18 is a bar graph showing that approx. 1% of antisense oligonucleotide variants increase editing at the target site compared to prototype constructs.

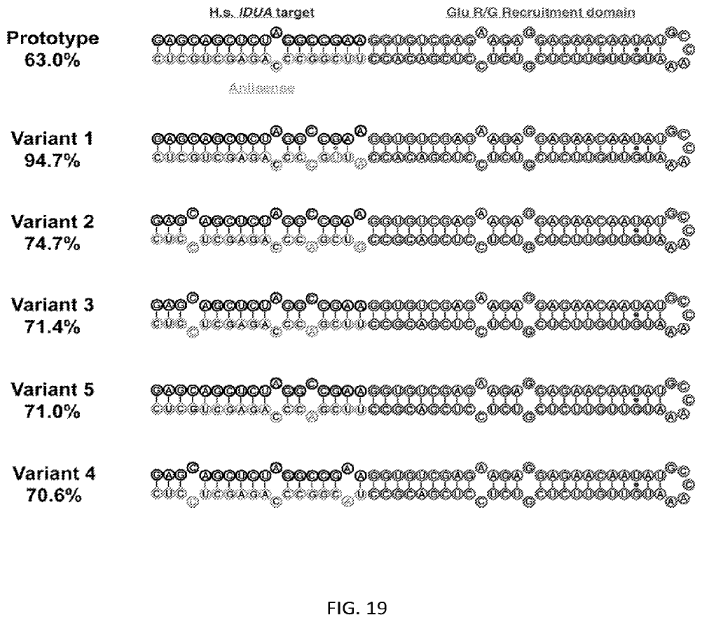

FIG. 19 shows antisense oligonucleotide variants containing modifications compared to the prototype.

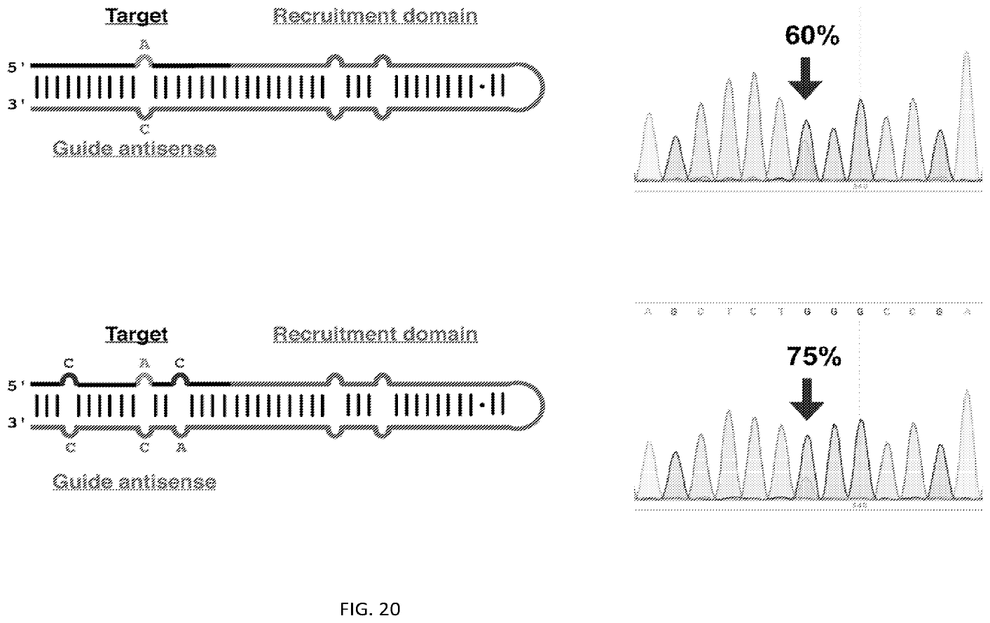

FIG. 20 shows validation of a highly edited variant identified in the screen (bottom left) by Sanger sequencing (bottom right); the prototype sequence (top left) and the corresponding editing level (top right) are also shown.

### Example 4

### Categorization of gRNA Variants With Enhanced Editing Efficacy

Following the methods described herein, various categories of mutations that enhance editing efficiency were identified. In particular, by screening >200,000 constructs targeting the human IDUA W402X mutation, the following features that enhance editing in target-ASO fusion. libraries were identified. We have also successfully applied the screening method to >10 other targets of therapeutic interest.

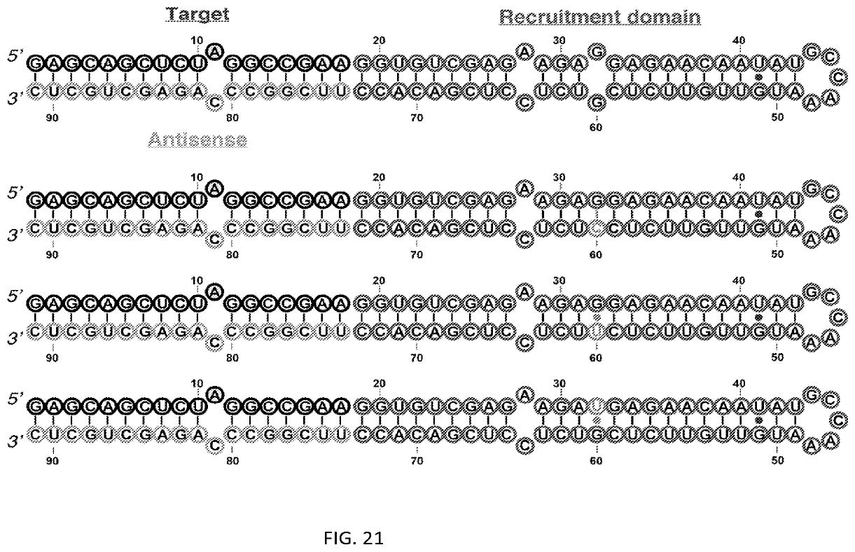

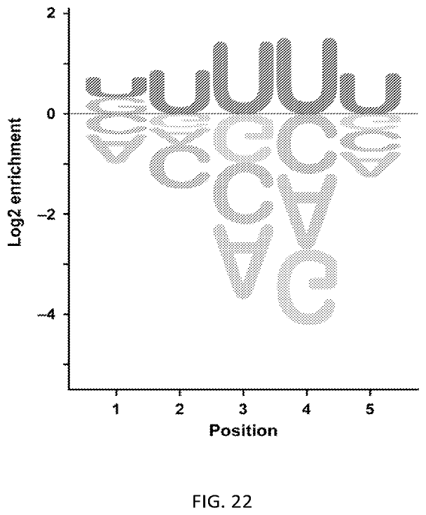

Category 1: Recruitment domain mutations. Because the recruitment domain constitutes a target-independent portion of the guide RNA, the below improvements should be universally applicable. Suitable mutations include replacing a mismatch in the original recruitment domain with a Watson-Crick or wobble base-pair (FIG. 21). Other suitable mutations include loop sequence mutations. 1015 of 1024 possible pentaloop sequences were screened, revealing a range of editing values of 44-95%. The top 10% of most highly edited sequences showed a strong enrichment for U-rich sequences, especially at loop positions 3 and 4 (FIG. 22).

Examples of guide sequences with Category 1 mutations are listed in Tables 1-3.

TABLE 1
The top 10% sequences of the recruitment
domain loop with the highest editing
levels. The recruitment domain stem and
the antisense region were kept constant.

| Loop sequence | % Edited |
|---|---|
| GAUUA (SEQ ID NO: 6) | 95.4 |
| GAUUG (SEQ ID NO: 7) | 94.3 |
| GGUUC (SEQ ID NO: 8) | 92.6 |
| GUUUG (SEQ ID NO: 9) | 92.0 |
| GUUUC (SEQ ID NO: 10) | 91.3 |
| UGAUU (SEQ ID NO: 11) | 91.1 |
| GUUUU (SEQ ID NO: 12) | 90.4 |
| GGUUA (SEQ ID NO: 13) | 90.2 |
| UUUCC (SEQ ID NO: 14) | 90.2 |
| UUUUU (SEQ ID NO: 15) | 90.1 |
| GCUUU (SEQ ID NO: 16) | 90.0 |
| AAUUG (SEQ ID NO: 17) | 90.0 |
| CCAUU (SEQ ID NO: 18) | 90.0 |
| CCUUU (SEQ ID NO: 19) | 89.6 |
| GGUUG (SEQ ID NO: 20) | 89.5 |
| UGUUU (SEQ ID NO: 21) | 89.1 |
| GCUUG (SEQ ID NO: 22) | 89.0 |
| CUUUA (SEQ ID NO: 23) | 88.7 |
| UCUUC (SEQ ID NO: 24) | 88.5 |
| GUUGA (SEQ ID NO: 25) | 88.4 |
| GUGUU (SEQ ID NO: 26) | 88.4 |
| UCUUU (SEQ ID NO: 27) | 88.3 |
| GGUUU (SEQ ID NO: 28) | 88.2 |
| GGAUU (SEQ ID NO: 29) | 88.1 |
| GUAUU (SEQ ID NO: 30) | 88.1 |
| UGUUC (SEQ ID NO: 31) | 88.0 |
| CUUUU (SEQ ID NO: 32) | 87.8 |
| UGUUG (SEQ ID NO: 33) | 87.8 |
| AUUUG (SEQ ID NO: 34) | 87.6 |
| GUCUU (SEQ ID NO: 35) | 87.4 |
| UUUCA (SEQ ID NO: 36) | 87.4 |
| UUCCC (SEQ ID NO: 37) | 87.2 |
| CGUUU (SEQ ID NO: 38) | 87.0 |
| ACUUU (SEQ ID NO: 39) | 87.0 |
| GAUUU (SEQ ID NO: 40) | 87.0 |
| UAUUU (SEQ ID NO: 41) | 86.8 |
| UUCAG (SEQ ID NO: 42) | 86.6 |
| AUUUC (SEQ ID NO: 43) | 86.6 |
| GUUGC (SEQ ID NO: 44) | 86.6 |
| UUUUA (SEQ ID NO: 45) | 86.4 |
| CAUUU (SEQ ID NO: 46) | 86.3 |
| UCUUA (SEQ ID NO: 47) | 86.2 |
| UUUUG (SEQ ID NO: 48) | 86.1 |
| CUUCC (SEQ ID NO: 49) | 86.0 |
| GUUUA (SEQ ID NO: 50) | 85.8 |
| CUUUC (SEQ ID NO: 51) | 85.8 |
| GUUAA (SEQ ID NO: 52) | 85.7 |
| AGUUG (SEQ ID NO: 53) | 85.6 |
| CUUUG (SEQ ID NO: 54) | 85.6 |
| CUCUU (SEQ ID NO: 55) | 85.6 |
| CACUU (SEQ ID NO: 56) | 85.6 |
| CGGUU (SEQ ID NO: 57) | 85.5 |
| GGGUU (SEQ ID NO: 58) | 85.5 |
| AGGUU (SEQ ID NO: 59) | 85.3 |
| GUUGU (SEQ ID NO: 60) | 85.2 |
| AAUUU (SEQ ID NO: 61) | 85.1 |
| UUUGU (SEQ ID NO: 62) | 85.0 |
| GGAAC (SEQ ID NO: 63) | 85.0 |
| CUGUU (SEQ ID NO: 64) | 84.9 |
| AUUUU (SEQ ID NO: 65) | 84.9 |
| AUUUA (SEQ ID NO: 66) | 84.9 |
| UUGAG (SEQ ID NO: 67) | 84.7 |
| AGUUC (SEQ ID NO: 68) | 84.6 |
| GUUGG (SEQ ID NO: 69) | 84.6 |
| CAAUU (SEQ ID NO: 70) | 84.6 |
| CUUGG (SEQ ID NO: 71) | 84.6 |
| UACUU (SEQ ID NO: 72) | 84.4 |
| AAUUC (SEQ ID NO: 73) | 84.3 |
| UUGAU (SEQ ID NO: 74) | 84.2 |
| GAUUC (SEQ ID NO: 75) | 84.0 |
| UAUUC (SEQ ID NO: 76) | 83.9 |
| AUUCA (SEQ ID NO: 77) | 83.9 |
| GUUCG (SEQ ID NO: 78) | 83.8 |
| UUUCU (SEQ ID NO: 79) | 83.5 |
| UUCUU (SEQ ID NO: 80) | 83.4 |
| CAGUU (SEQ ID NO. 81) | 83.3 |
| UAUUG (SEQ ID NO: 82) | 83.3 |
| UGUUA (SEQ ID NO: 83) | 83.3 |
| UUAUU (SEQ ID NO: 84) | 83.3 |
| UUUAA (SEQ ID NO: 85) | 83.2 |
| UGGUU (SEQ ID NO: 86) | 83.1 |
| AAGUU (SEQ ID NO: 87) | 83.1 |
| GUCCC (SEQ ID NO: 88) | 83.0 |
| GACUU (SEQ ID NO: 89) | 82.8 |
| UUUCG (SEQ ID NO: 90) | 82.7 |
| UUUGG (SEQ ID NO: 91) | 82.6 |
| GAGUU (SEQ ID NO: 92) | 82.6 |
| CCUUA (SEQ ID NO: 93) | 82.6 |
| GUUAC (SEQ ID NO: 94) | 82.5 |
| AUUGA (SEQ ID NO: 95) | 82.5 |
| GCUUA (SEQ ID NO: 96) | 82.5 |
| AGCUU (SEQ ID NO: 97) | 82.4 |
| AGUUA (SEQ ID NO: 98) | 82.4 |
| UCCAU (SEQ ID NO: 99) | 82.4 |
| GGUCG (SEQ ID NO: 100) | 82.4 |
| CCUUC (SEQ ID NO: 101) | 82.3 |
| CCGUU (SEQ ID NO: 102) | 82.2 |
| UUUGC (SEQ ID NO: 103) | 82.2 |
| UUGAA (SEQ ID NO: 104) | 82.1 |
| UCAUU (SEQ ID NO: 105) | 81.8 |
| CUUGA (SEQ ID NO: 106) | 81.8 |
| AAAUU (SEQ ID NO: 107) | 81.8 |

TABLE 2
Examples of guide sequences with optimized
sequence of the 5′ strand of the recruitment
domain stem. Sequences with greater than 5%
change in editing level above the prototype
design (FIG. 23A; 67.3% editing) are shown
and sequence changes relative to the proto-
type sequence are indicated (see FIG. 23A
for numbering). The 3′ strand of the re-
cruitment domain, the loop, and the anti-
sense region were kept constant.

| | Sequence | |
|---|---|---|
| | changes | |
| | relative | |
| Sequence of the 5′ strand | to pro- | % |
| of the recruitment domain | totype | Edited |
| GGUGGCUAGAAGAGGAGAGCAAAAU | 5G, 7U, | 96.0 |
| (SEQ ID NO: 108) | 19G, 23A | |
| GGAGUCGAGAUGAGGAGGGCCAUAU | 3A, 11U, | 91.8 |
| (SEQ ID NO: 109) | 18G, 19G, | |
| | 21C | |
| GAUGUCGAGAAGAGGAGAACUAUAU | 2A, 21U | 89.2 |
| (SEQ ID NO: 110) | | |
| GGUGUUAAGAAGAGGAGAACAAUUU | 6U, 7A, | 88.0 |
| (SEQ ID NO: 111) | 24U | |
| GGUGACGAGAAGAUGAGAACAAUAG | 5A, 14U, | 85.7 |
| (SEQ ID NO: 112) | 25G | |
| GGUGUGGAGAAGAGGAGAACACUAU | 6G, 22C | 84.8 |
| (SEQ ID NO: 113) | | |
| GAUGUCUAGAAGAGGAGAACACUAU | 2A, 7U, | 83.3 |
| (SEQ ID NO: 114) | 22C | |
| GGUCUGGAGAACAGGAGAACAAUAU | 4C, 6G, | 82.0 |
| (SEQ ID NO: 115) | 12C | |
| GGAGUCGAGCAGAGGAAAACCAUAU | 3A, 10C, | 81.8 |
| (SEQ ID NO: 116) | 17A, 21C | |
| GAUGUCUAGAAGAGGAGAACAAUGU | 2A, 7U, | 81.6 |
| (SEQ ID NO: 117) | 24G | |
| GGUUUCGGGAAGAGGAGAACAAUAG | 4U, 8G, | 81.5 |
| (SEQ ID NO: 118) | 25G | |
| GGUGCCGAGAAGACGAGAACAAUAA | 5C, 14C, | 81.4 |
| (SEQ ID NO: 119) | 25A | |
| GAUGUCGAGAAGAUGAGAACAAUAU | 2A, 14U | 80.6 |
| (SEQ ID NO: 120) | | |
| GGUGUCGAGAAGCCGAGAACAAUAU | 13C, 14C | 80.0 |
| (SEQ ID NO: 121) | | |
| GAUGUCGAGAAGUGGAGAACAAUAU | 2A, 13U | 80.0 |
| (SEQ ID NO: 122) | | |
| GGUAUCAAGAAGAUGAGAACAAUAA | 4A, 7A, | 79.4 |
| (SEQ ID NO: 123) | 14U, 25A | |
| GGUGACGAGAAGUUGAGAACAAUAU | 5A, 13U, | 79.2 |
| (SEQ ID NO: 124) | 14U | |
| GGCGUCGAGAAGAGGAGAACAAGGU | 3C, 23G, | 78.6 |
| (SEQ ID NO: 125) | 24G | |
| GGUGUUGAGAAGACGAGAACAAUAU | 6U, 14C | 76.7 |
| (SEQ ID NO: 126) | | |
| GGUGUCGAGAAGUUGAGAACAAUCU | 13U, 14U, | 76.2 |
| (SEQ ID NO: 127) | 24C | |
| GGUGUCGCGAAGCGGAGAACAUUAU | 8C, 13C, | 76.1 |
| (SEQ ID NO: 128) | 22U | |
| GUUGUCGAGAAGGGGAGAACAAUAU | 2U, 13G | 75.8 |
| (SEQ ID NO: 129) | | |
| GGUGUCGAGAAGAUGAGAACAAUAU | 14U | 75.6 |
| (SEQ ID NO: 130) | | |
| GGUGUCGAGAAUACGAGAAAAAUUG | 12U, 14C, | 75.0 |
| (SEQ ID NO: 131) | 20A, 24U, | |
| | 25G | |
| GUUUUCGACAAGAGGCAAACAUUGU | 2U, 4U, | 75.0 |
| (SEQ ID NO: 132) | 9C, 16C, | |
| | 17A, 22U, | |
| | 24G | |
| GGUGUGGAGAAGACGAGAGCAAUUU | 6G, 14C, | 74.3 |
| (SEQ ID NO: 133) | 19G, 24U | |
| GGUGUCGAGAAGAGGAGAACAAUAG | 25G | 74.0 |
| (SEQ ID NO: 134) | | |
| GAUGUCGAGAAGAGGAGAACAAUUU | 2A, 24U | 73.7 |
| (SEQ ID NO: 135) | | |
| GGUGUCGAGAAGAGGAGAACAACAU | 23C | 73.6 |
| (SEQ ID NO: 136) | | |
| GGUGUCGAGAAGAGGAGAAUAACAU | 20U, 23C | 73.3 |
| (SEQ ID NO: 137) | | |
| GAUGUCGAGAAUAUGAGAACAAUAU | 2A, 12U, | 73.2 |
| (SEQ ID NO: 138) | 14U | |
| GAUGUCGAGAAGAGGAGAACAAUAU | 2A | 73.1 |
| (SEQ ID NO: 139) | | |
| GGUGAUGAGAAGAGGAGAACAAUAU | 5A, 6U | 73.1 |
| (SEQ ID NO: 140) | | |
| GAUGUCGAGAAGAGGAGAACAAUAA | 2A, 25A | 72.9 |
| (SEQ ID NO: 141) | | |
| GAUGUCUAGAAGAUGAGAACAAUAU | 2A, 7U, | 72.4 |
| (SEQ ID NO: 142) | 14U | |
| GGUGUCGAGAAGAUGAGAACAAUGU | 14U, 24G | 72.3 |
| (SEQ ID NO: 143) | | |

TABLE 3
Examples of guide sequences with optimized
sequence of the 3′ strand of the recruitment
domain stem. Sequences with greater than 5%
change in editing level above the prototype
design (FIG. 23B; 63.0% editing) are shown
and sequence changes relative to the proto-
type sequence are indicated (see FIG. 23B
for numbering). The 5′ strand of the re-
cruitment domain, the loop, and the anti-
sense region were kept constant.

| | Sequence | |
|---|---|---|
| | changes | |
| | relative | |
| Sequence of the 3' strand | to pro- | % |
| of the recruitment domain | totype | Edited |
| AUGUUGUGCUCGUCUCCUCGACGCC | 8G, 23G | 100.0 |
| (SEQ ID NO: 144) | | |
| AUGUUCUUCUCGUCUGCUGGACACU | 6C, 16G, | 93.8 |
| (SEQ ID NO: 145) | 19G, 25U | |
| AUGUUGUUCUCGCCCCCUCGGCACC | 13C, 15C, | 91.3 |
| (SEQ ID NO: 146) | 21G | |
| AUGUUGUUCUCCUCUCCUCGACACC | 12C | 88.2 |
| (SEQ ID NO: 147) | | |
| AGGUUGUUCUCGUCUCCUCAACACC | 2G, 20A | 85.7 |
| (SEQ ID NO: 148) | | |
| GUGUUGUUCUCGUCUCCGCGACAAC | 1G, 18G, | 85.0 |
| (SEQ ID NO: 149) | 24A | |
| AUGUUGUUCUCCUCUUCUCGACACC | 12C, 16U | 83.4 |
| (SEQ ID NO: 150) | | |
| AUGUUGUUCUCCUCUCGUCGACAUC | 12C, 17G, | 80.5 |
| (SEQ ID NO: 151) | 24U | |
| AUGCUGUUCUCCUCUCCUCGACACC | 4C, 12C | 80.5 |
| (SEQ ID NO: 152) | | |
| AUGUUGUUCUCCUCUCUUCGACACC | 12C, 17U | 79.2 |
| (SEQ ID NO: 153) | | |
| AUAUUGCUCUCGUCUCCUGGUCACC | 3A, 7C, | 76.9 |
| (SEQ ID NO: 154) | 19G, 21U | |
| AUGUUGUUCUUCUCUCCUCGACACC | 11U, 12C | 76.5 |
| (SEQ ID NO: 155) | | |
| GUGUUGUUCUCGUCUCCUCAACACC | 1G, 20A | 75.0 |
| (SEQ ID NO: 156) | | |
| AACUUGUUCUCGUCUCCUCCACACC | 2A, 3C, | 75.0 |
| (SEQ ID NO: 157) | 20C | |
| AUGAUGUUCUCGUCUGCUCGACCCC | 4A, 16G, | 75.0 |
| (SEQ ID NO: 158) | 23C | |
| GUGUUGUUCUCGUCUCCUCGACAGC | 1G, 24G | 74.4 |
| (SEQ ID NO: 159) | | |
| AUGUUGUUCUCUUCUCCUCGACACC | 12U | 74.1 |
| (SEQ ID NO: 160) | | |
| AUGUUGCUCUCCUCUCCUCGAGACC | 7C, 12C, | 73.8 |
| (SEQ ID NO: 161) | 22G | |
| ACGUUGUUCUCUUCUCCUCGACACC | 2C, 12U | 73.4 |
| (SEQ ID NO: 162) | | |
| AUGUUGUUCUCAUCUCCUCGACAGC | 12A, 24G | 72.7 |
| (SEQ ID NO: 163) | | |
| AUGUUGUCCUCCUCUCCUCGACACC | 8C, 12C | 72.4 |
| (SEQ ID NO: 164) | | |
| AUGCUGUUCUCGUCUCCUCGGCACC | 4C, 21G | 72.0 |
| (SEQ ID NO: 165) | | |
| AUCUUGUUCUCGUCUCCUCGACAUC | 3C, 24U | 71.7 |
| (SEQ ID NO: 166) | | |
| AGGUUGUUCUCGUCUCCUCGACAUC | 2G, 24U | 71.4 |
| (SEQ ID NO: 167) | | |
| UGGUUGUUCUCGUCUCCUAGACAAC | 1U, 2G, | 71.4 |
| (SEQ ID NO: 168) | 19A, 24A | |
| GUGUUGUUCUCGUCUCCUCGAUACC | 1G, 22U | 71.1 |
| (SEQ ID NO: 169) | | |
| UUGUUGUCCUCGUCUCCUCGACACC | 1U, 8C | 70.8 |
| (SEQ ID NO: 170) | | |
| CUGUUGUUCUCGUCUCCUCGACACC | 1C | 70.8 |
| (SEQ ID NO: 171) | | |
| GUGUUGUUCUCGUCUCCUCGAGACC | 1G, 22G | 70.5 |
| (SEQ ID NO: 172) | | |
| UGGUUGUUCUCGUCUCCUCGAAAAC | 1U, 2G, | 70.4 |
| (SEQ ID NO: 173) | 22A, 24A | |
| GUGUUGUUCUCGUCUGCUCGACACC | 1G, 16G | 70.2 |
| (SEQ ID NO: 174) | | |
| AUGUUGUUCUCUUCUCCUCAACACC | 12U, 20A | 70.1 |
| (SEQ ID NO: 175) | | |
| UUGUUGUUCUCGCCUCCUCGAGACC | 1U, 13C, | 69.8 |
| (SEQ ID NO: 176) | 22G | |
| AUGUUGUUCUCAUCUACUCGACAUC | 12A, 16A, | 69.6 |
| (SEQ ID NO: 177) | 24U | |
| AUGUUGUUCUCUUCUCCUCGAGACC | 12U, 22G | 69.6 |
| (SEQ ID NO: 178) | | |
| CUAUUGUUCUCGUCUCCUCGACACC | 1C, 3A | 69.5 |
| (SEQ ID NO: 179) | | |
| AUGUUGUUCUCUUCUCCUCGGCCUC | 12U, 21G, | 69.5 |
| (SEQ ID NO: 180) | 23C, 24U | |
| AUGUUGUUCUCCUCCCCUCGACACC | 12C, 15C | 69.4 |
| (SEQ ID NO: 181) | | |
| AUGUUGUUCUCACCUUCUCGACACC | 12A, 13C, | 69.4 |
| (SEQ ID NO: 182) | 16U | |
| AGGUUGUUCUCGUCUCCUUGUCACC | 2G, 19U, | 69.4 |
| (SEQ ID NO: 183) | 21U | |
| AUGUUGUUCUCCUCUCCUUGACACC | 12C, 19U | 69.4 |
| (SEQ ID NO: 184) | | |
| AGGUUGUUCUCGUCUCCUCGACACC | 2G | 69.3 |
| (SEQ ID NO: 185) | | |
| AUGUUGUUCUCGUCUCCUCGACGGC | 23G, 24G | 69.3 |
| (SEQ ID NO: 186) | | |
| CUGUUGUUCUCUUCUCCUCGACACC | 1C, 12U | 69.0 |
| (SEQ ID NO: 187) | | |
| AUGUUGUUCUCGUCUCCUGGCCACC | 19G, 21C | 69.0 |
| (SEQ ID NO: 188) | | |
| AUGUUGUUCUCCUCUCCACGAAUCC | 12C, 18A, | 68.8 |
| (SEQ ID NO: 189) | 22A, 23U | |
| GUGUUGAUCUCUUCUCCUCGACACC | 1G, 7A, | 68.4 |
| (SEQ ID NO: 190) | 12U | |
| UUGUUGUUCUCGUCUCCUCGAGACC | 1U, 22G | 68.2 |
| (SEQ ID NO: 191) | | |
| AUGUUGUUCUCGUCUCCUCGAACCC | 22A, 23C | 68.2 |
| (SEQ ID NO: 192) | | |
| ACGUUGUUCUCGUCUCCUCGAAAUC | 2C, 22A, | 68.2 |
| (SEQ ID NO: 193) | 24U | |
| AUGUUGUUCUCGUCUCCUGGACUCC | 19G, 23U | 68.1 |
| (SEQ ID NO: 194) | | |

Category 2: Mismatches in the target:antisense duplex. Mismatches and wobble base-pairs in the antisense region can enhance editing of the IDUA W402X target (Tables 4-6). Certain mismatches or combinations thereof are enriched in antisense variants that give the most efficient editing (FIG. 19). The positions of beneficial mismatches relative to the editing site appear to be independent of variation in the length of the target:antisense duplex and of the recruitment domain, such as when the target:antisense duplex is extended by 5 bp upstream or downstream (FIG. 23D, E). The same beneficial mismatch positions (relative to the target site) persist when the hIDUA editing site is shifted by 5 bp towards the 5′ end or when the recruitment domain is replaced with downstream IDUA sequence.

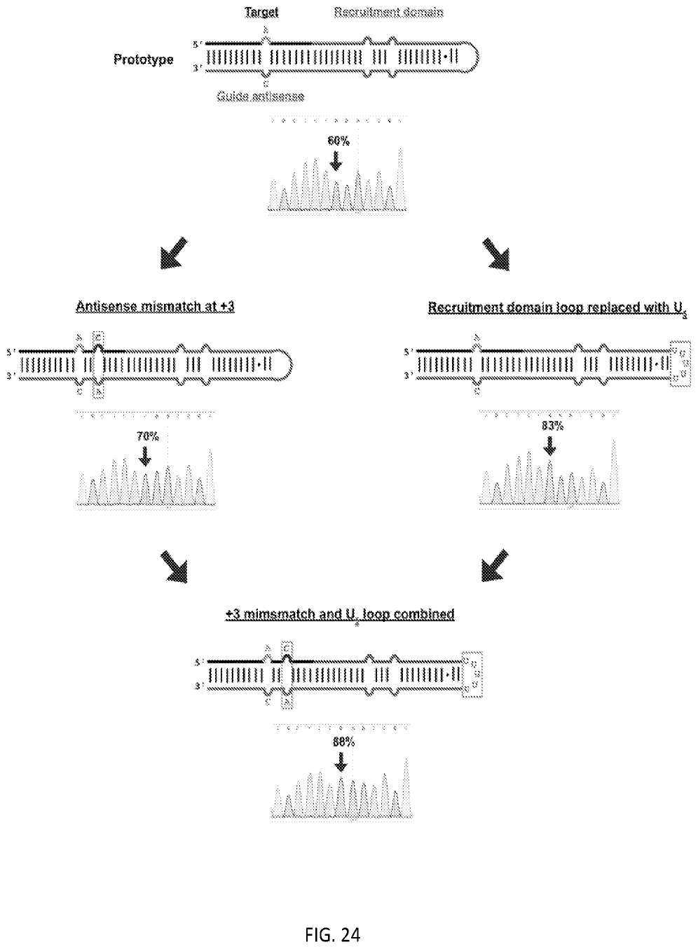

Combinations of individual guide features, such as a combination of a mismatch in the antisense region and a substitution of the recruitment domain loop, or a combination of several mismatches in the antisense region, tend to have additive effects on editing (FIG. 24). In trans guides, these additive effects should be balanced with potential destabilizing effects of multiple mutations on guide/target binding.

TABLE 4
Examples of guide sequences with optimized
antisense domains. Sequences with greater
than 5% change in editing level above the
prototype design (63.0%) are shown and
sequence changes relative to the prototype
sequence are indicated (see FIG. 23C for
numbering). The recruitment domain was
kept constant. Only variants that showed
no greater than 5% relative standard
deviation between biologicalreplicates
are shown.

| | Sequence | |
|---|---|---|
| | changes | |
| | relative | |
| | to pro- | % |
| Antisense sequence | totype | Edited |
| AUUGCCCCAGAGCUGCUC | +7A, +5U, | 94.7 |
| (SEQ ID NO: 195) | +3C | |
| GUCGACCCAGAGCUCCUC | +7G, +3A, | 74.7 |
| (SEQ ID NO: 196) | −7C | |
| UUCGACCCAGAGCUCCUC | +3A, −7C | 71.4 |
| (SEQ ID NO: 197) | | |
| UUCGACCCAGAGCUGCUC | +3A | 71.0 |
| (SEQ ID NO: 198) | | |
| UACGGCCCAGAGCUCCUC | +6A, −7C | 70.6 |
| (SEQ ID NO: 199) | | |
| UUCGACCCAAAGCUGCUC | +3A, −2A | 69.7 |
| (SEQ ID NO: 200) | | |
| UUAGACCCAGAGCUUCUC | +5A, +3A, | 68.5 |
| (SEQ ID NO: 201) | −7U | |

TABLE 5
Examples of guide sequences with optimized antisense sequences
from a library in which the target-antisense duplex was ex-
tended by 5 bp at the 5′ end of the target sequence. Sequences
with greater than 5% change in editing level above the proto-
type design (FIG. 23D; edited at 56.6%) are shown and sequence
changes relative to the prototype sequence are indicated (see
FIG. 23D for numbering). The recruitment domain was kept constant.

| | Sequence changes | |
|---|---|---|
| | relative to | % |
| Antisense sequence | prototype | Editing |
| GUCGACCCAGAGCUGCUCAUCAU (SEQ ID NO: 202) | +7G, +3A, −11A | 98.5 |
| UUCGGUGCAGAGCUGCUCCUCAU (SEQ ID NO: 203) | +2U, +1G | 87.5 |
| UUCGCCCCAGAGCUGCUCCCCAA (SEQ ID NO: 204) | +3C, −12C, −15A | 86.7 |
| UUCGACCCAGAGCUGCUCCUGAA (SEQ ID NO: 205) | +3A, −13G, −15A | 79.0 |
| UUCGCCCCCGAGCAGCACGUCAU (SEQ ID NO: 206) | +3C, −1C, −6A, −9A, | 78.6 |
| | −11G | |
| UACGGCCCAGAGCUCCUCCUCGU (SEQ ID NO: 207) | +6A, −7C, −14G | 75.7 |
| UGAGGCCCAGAGCUCCUCCUCAC (SEQ ID NO: 208) | +6G, +5A, −7C, −15C | 75.0 |
| UUCGGCCCAGAGUUGCUCCCCAU (SEQ ID NO: 209) | −5U, −12C | 74.7 |
| UCCGGUCCAGAGCUGUUCCUCAU (SEQ ID NO: 210) | +6C, +2U, −8U | 74.1 |
| UUCGGCCCAGAGGUGCUCGUCAU (SEQ ID NO: 211) | −5G, −11G | 73.3 |
| UUCGGCCCGGAGCUGCUGCUCAU (SEQ ID NO: 212) | −1G, −10G | 73.3 |
| UUCGCUCCAGAGCUGCUCCUCAA (SEQ ID NO: 213) | +3C, +2U, −15A | 72.6 |
| GUUGGCCCAGAUCUUCUCCUCAU (SEQ ID NO: 214) | +7G, +5U, −4U, −7U | 72.4 |
| UGCGGUCCAGAGCUGCUCCUCAU (SEQ ID NO: 215) | +6G, +2U | 72.4 |
| CUCGGCCCAGAGCUGACCCUCAU (SEQ ID NO: 216) | +7C, −8A, −9C | 71.4 |
| UUCGGCCCAGAGCUGCUCCUCCC (SEQ ID NO: 217) | −14C, −15C | 71.1 |
| CACGGCCCAGAGCUGCUCCUCAU (SEQ ID NO: 218) | +7C, +6A | 71.0 |
| UACGGCCCAGAGCUCCUCCUCAU (SEQ ID NO: 219) | +6A, −7C | 70.7 |
| UUCGGCCCAGAGCUUCUUCUCAU (SEQ ID NO: 220) | −7U, −10U | 69.5 |
| UUGGGCCCAGAGCUGCUCCUCUU (SEQ ID NO: 221) | +5G, −14U | 69.1 |
| UUCGGCCCAGAGCUCCUCCUAAU (SEQ ID NO: 222) | −7C, −13A | 69.0 |
| UUCGACCCAGAGCUGCUUCUCAU (SEQ ID NO: 223) | +3A, −10U | 68.3 |
| UUCGGCCCAGAGCUCCUCCUUAU (SEQ ID NO: 224) | −7C, −13U | 68.1 |
| UUCGACCCAGAGCUGCCCCUCAU (SEQ ID NO: 225) | +3A, −9C | 68.1 |
| CUCGGCCCAGAGCUUCUCCUCAU (SEQ ID NO: 226) | +7C, −7U | 68.0 |
| UUCGACCCAGAGCUGCUCCUCCG (SEQ ID NO: 227) | +3A, −14C, −15G | 67.8 |
| UUGGGCCCAGAGCUGCCCCUCAU (SEQ ID NO: 228) | +5G, −9C | 67.4 |
| UCCGGCCCAGAGCUCCUCCUCAU (SEQ ID NO: 229) | +6C, −7C | 67.4 |
| UUGGGCCCAGAGCUCCUCCUCAU (SEQ ID NO: 230) | +5G, −7C | 67.3 |
| UUCGGCCCAGAGCUCCUCUUCAU (SEQ ID NO: 231) | −7C, −11U | 67.3 |
| UUAGGCCCAGAGCUCCUCCUCAA (SEQ ID NO: 232) | +5A, −7C, −15A | 67.1 |
| UUCGACCCAGAGCUGCUCAUCAU (SEQ ID NO: 233) | +3A, −11A | 67.1 |
| GUCGCCCCAGAGCUGCUCGUCAU (SEQ ID NO: 234) | +7G, +3C, −11G | 66.7 |
| UUCGGCCCAGUGCUGCUCCUUAU (SEQ ID NO: 235) | −3U, −13U | 66.7 |
| UUCAGCCCAAAGCUGCUCCUCAU (SEQ ID NO: 236) | +4A, −2A | 66.7 |
| UUCGCCCCAGAGCUGCUCCUAAU (SEQ ID NO: 237) | +3C, −13A | 66.7 |
| UUCUGCCCAGAGCUGCUCUUCAU (SEQ ID NO: 238) | +4U, −11U | 66.7 |
| UUCGGGCCAGAGCUCCUCCUCAU (SEQ ID NO: 239) | +2G, −7C | 66.7 |
| UCCGGCCCAGAGCUGCUCCUUAU (SEQ ID NO: 240) | +6C, −13U | 66.7 |
| UGCGGCCCAGAGCUGCUCCUCUA (SEQ ID NO: 241) | +6G, −14U, −15A | 66.5 |
| UUCGACCCAGAGCUGCUCCUUAU (SEQ ID NO: 242) | +3A, −13U | 66.4 |
| UUCGACCCAGAGCUGAUCCUCAU (SEQ ID NO: 243) | +3A, −8A | 66.3 |
| UUCGACCCAGAGCUGCUCCCCAG (SEQ ID NO: 244) | +3A, −12C, −15G | 66.2 |
| UUCGGCCCAGAGCUGCUUCUAUU (SEQ ID NO: 245 | −10U, −13A, −14U | 66.1 |
| GACGGCCCAGAGCUGCUCAUCAU (SEQ ID NO: 246) | +7G, +6A, −11A | 66.0 |
| UUCGGCCCAGAGCUGCUUCUCUA (SEQ ID NO: 247) | −10U, −14U, −15A | 65.8 |
| UGCGGCCCAGAGCUGCUCCGCAU (SEQ ID NO: 248) | +6G, −12G | 65.8 |
| UUCGCCCCAGAGCUGCUCCCCAU (SEQ ID NO: 249) | +3C, −12C | 65.7 |
| UUCGGCCCAGAGCUUCUCCUAAU (SEQ ID NO: 250) | −7U, −13A | 65.6 |
| UUCUGUCCAGAGCUGCUCCUCAU (SEQ ID NO: 251) | +4U, +2U | 65.4 |
| UUCGGCCCAGAGCUGCCCCCCAU (SEQ ID NO: 252) | −9C, −12C | 65.4 |
| UUCUACCCAGAGCUGCUCCUCAU (SEQ ID NO: 253) | +4U, +3A | 65.4 |
| UUGGGUCCAGAGCUGCUCCUCAU (SEQ ID NO: 254) | +5G, +2U | 65.3 |
| UUCGGCCCAGAGCUGCUCCUACU (SEQ ID NO: 255) | −13A, −14C | 65.3 |
| UUCGUCCCAGAGCUGCCCCUCAU (SEQ ID NO: 256) | +3U, −9C | 65.2 |
| UUCGGCCCAGAGCUACUCAUCAU (SEQ ID NO: 257) | −7A, −11A | 65.2 |
| UUCGACCCAGAGCUGCUCCUCAA (SEQ ID NO: 258) | +3A, −15A | 65.1 |
| CUCGGCCCAGAGCUGCUCCUCUU (SEQ ID NO: 259) | +7C, −14U | 65.1 |
| UUCGGCGCAGGGCUGCUCCUCAU (SEQ ID NO: 260) | +1G, −3G | 65.0 |
| UUCGGCCCAGAUCUUCUCCUUAU (SEQ ID NO: 261) | −4U, −7U, −13U | 65.0 |
| AUCGACCCAGAGCUGCUCCUCAU (SEQ ID NO: 262) | +7A, +3A | 64.8 |
| UACGACCCAGAGCUGCUCCUCAA (SEQ ID NO: 263) | +6A, +3A, −15A | 64.8 |
| UGCGACCCAGAGCUGCUCCUCAU (SEQ ID NO: 264) | +6G, +3A | 64.4 |
| UUCGGCCCAGAGCUCCUCCUCAC (SEQ ID NO: 265) | −7C, −15C | 64.4 |
| GUCGGUCCAGAGGUGCUCCUCAU (SEQ ID NO: 266) | +7G, +2U, −5G | 64.3 |
| UUCGGCCCAGAGCUGCUUCUCAG (SEQ ID NO: 267) | −10U, −15G | 64.2 |
| UUCGACCCAGAGCUGCUAAUCAU (SEQ ID NO: 268) | +3A, −10A, −11A | 64.0 |
| UUAGGUCCAGAGCUGCUACUCAU (SEQ ID NO: 269) | +5A, +2U, −10A | 63.9 |
| UUAGGCCCAGAGCUCCUCCUCAU (SEQ ID NO: 270) | +5A, −7C | 63.9 |
| UUGGGCCCAGAGCUGCUCCUCAA (SEQ ID NO: 271) | +5G, −15A | 63.8 |
| UUCGACCCAGAGCUGCUCCUCAU (SEQ ID NO: 272) | +3A | 63.8 |
| UUCGACCCAGAGCUGCUACUCAU (SEQ ID NO: 273) | +3A, −10A | 63.7 |
| UUCGGCCCAGAGCUGGUCCACAU (SEQ ID NO: 274) | −8G, −12A | 63.6 |
| CUCGGCCCAGAGCUGAUCCUCAU (SEQ ID NO: 275) | +7C, −8A | 63.6 |
| UUCGGCCCAGAGCUCCUGCUCAU (SEQ ID NO: 276) | −7C, −10G | 63.6 |
| UUCGGCCCAGAGCUGCUCCCCUU (SEQ ID NO: 277) | −12C, −14U | 63.5 |
| UUCGGCCCAGAGCUCCUCCGCAU (SEQ ID NO: 278) | −7C, −12G | 63.4 |
| UGCGGCCCAGAGCUGCUUCUCAC (SEQ ID NO: 279) | +6G, −10U, −15C | 63.3 |
| UUCGGCCCAGAGCUCCUCCACAU (SEQ ID NO: 280) | −7C, −12A | 63.3 |
| UUCGGCCCAGAGCUGCUUUUCAU (SEQ ID NO: 281) | −10U, −11U | 63.2 |
| UUCGACCCAGAGCUCCUCCUCAA (SEQ ID NO: 282) | +3A, −7C, −15A | 63.2 |
| GACGACCCAGAGCUGCUCCUCAU (SEQ ID NO: 283) | +7G, +6A, +3A | 63.1 |
| UGCGACCCAGAGCUGCUCCCCAU (SEQ ID NO: 284) | +6G, +3A, −12C | 63.0 |
| UUCGGCCCAGAGCUCCUCCUCAU (SEQ ID NO: 285) | −7C | 62.9 |
| UUCUGCCCAGAGCUGCUCGUCAU (SEQ ID NO: 286) | +4U, −11G | 62.9 |
| UGCGGCCCAGAACUCCUCCUCAU (SEQ ID NO: 287) | +6G, −4A, −7C | 62.7 |
| UUCGGCCCAAAGCUGCUCCCCAU (SEQ ID NO: 288) | −2A, −12C | 62.6 |
| UUCGGUCCAGAGCUCCUCCUCAU (SEQ ID NO: 289) | +2U, −7C | 62.6 |
| UUCGUCCCAGAGCUGCUCCUCAG (SEQ ID NO: 290) | +3U, −15G | 62.5 |
| UUCGGCCCAGAUCUGCUCCUCCU (SEQ ID NO: 291) | −4U, −14C | 62.5 |
| UUCGGCCCAGAGCACCUCCUCAU (SEQ ID NO: 292) | −6A, −7C | 62.5 |
| UUCGGCGCAGAGGUGCUCCUCAU (SEQ ID NO: 293) | +1G, −5G | 62.5 |
| UUCGGCCCAGAGCUUACCCUCUU (SEQ ID NO: 294) | −7U, −8A, −9C, −14U | 62.5 |
| UUCGGCCCAGAGCUGAUCUUCAU (SEQ ID NO: 295) | −8A, −11U | 62.5 |
| UUAUGCCCAGAGCUGCUCCUCAU (SEQ ID NO: 296) | +5A, +4U | 62.2 |
| UUCGGCCCAGAGGUGCUCUUCAU (SEQ ID NO: 297) | −5G, −11U | 62.0 |
| CUCGGCCCAGAGCUCCUCCUCAU (SEQ ID NO: 298) | +7C, −7C | 61.9 |
| UUCGGCCCAGAGCUCCGCCUCAU (SEQ ID NO: 299) | −7C, −9G | 61.8 |
| UUCGGCCCAGAGGUCCUCCUCUU (SEQ ID NO: 300) | −5G, −7C, −14U | 61.7 |
| UUCGGCCCAGAGGUGCUCCUCUU (SEQ ID NO: 301) | −5G, −14U | 61.7 |
| UGCGGCCCAGAGCUGCUCCUCAU (SEQ ID NO: 302) | +6G | 61.6 |

TABLE 6
Examples of guide sequences with optimized antisense sequences
from a library in which the target-antisense duplex was ex-
tended by 5 bp at the 3′ end of the target sequence. Sequences
with greater than 5% change in editing level above the proto-
type design (FIG. 23E; edited at 56.0%) are shown and sequence
changes relative to the prototype sequence are indicated (see
FIG. 23E for numbering). The recruitment domain was kept constant.

| | Sequence changes | |
|---|---|---|
| | relative to | % |
| Antisense sequence | prototype | Editing |
| GACGCAUCCGCCCAGAGCUACUC (SEQ ID NO: 303) | +9G, +7A, +4C, −7A | 95.6 |
| CACACUUCGGGCCAGAGCAACUC (SEQ ID NO: 304) | +12C, +2G, −6A, −7A | 93.8 |
| GACGCUUGGGCCCAGAGCUGCUC (SEQ ID NO: 305) | +9G, +5G | 87.3 |
| GACGCUGCGGCCCAGAGCUGCUC (SEQ ID NO: 306) | +9G, +6G | 86.1 |
| GGCACCUCGGCCCAGAGCUGCUC (SEQ ID NO: 307) | +11G, +7C | 84.9 |
| CACACUUCUGCCCAGAGCUGCUC (SEQ ID NO: 308) | +12C, +4U | 84.6 |
| GAGGCUUCGGCCCAGAGCUGCUC (SEQ ID NO: 309) | +10G, +9G | 82.8 |
| GACGCUUCCGCCCAGAGCUGCUC (SEQ ID NO: 310) | +9G, +4C | 79.4 |
| GAGCCUUCGACCCAGAGCUGCUC (SEQ ID NO: 311) | +10G, +9C, +3A | 77.6 |
| GACACUUCGGUCCAGAGCUCCUC (SEQ ID NO: 312) | +2U, −7C | 77.6 |
| GACUCUUCGGCCCAGGGCUCCUC (SEQ ID NO: 313) | +9U, −3G, −7C | 76.9 |
| GACGCUUCGACCCAGAGCUGCUC (SEQ ID NO: 314) | +9G, +3A | 75.8 |
| GUCACCUCGACCCAGAGCUGCUC (SEQ ID NO: 315) | +11U, +7C, +3A | 75.6 |
| GACCCUUUGACCCAGAGCUGCUC (SEQ ID NO: 316) | +9C, +5U, +3A | 75.0 |
| GACCCUUCGGCCCAGAGCUCCUC (SEQ ID NO: 317) | +9C, −7C | 74.5 |
| GGCACUUCGACCCAGAGCUGCUC (SEQ ID NO: 318) | +11G, +3A | 74.4 |
| GCCACUUCGACCCAGAGCUGCUC (SEQ ID NO: 319) | +11C, +3A | 74.4 |
| GACGCUUCAGCCCAGAGCUGCUC (SEQ ID NO: 320) | +9G, +4A | 74.3 |
| GACAAUUCGGUCCAGAGCUGCUC (SEQ ID NO: 321) | +8A, +2U | 73.7 |
| GACGCUUCGGCCCAGUGCUGCUC (SEQ ID NO: 322) | 19G, −3U | 73.6 |
| GGCACUUCGUCCCAGAGCUGCUC (SEQ ID NO: 323) | +11G, +3U | 73.2 |
| GAAACAUCGACCCAGAGCUGCUC (SEQ ID NO: 324) | +10A, +7A, +3A | 72.9 |
| GACAUUUCGACCCAGAGCUGCUC (SEQ ID NO: 325) | +8U, +3A | 72.9 |
| GGCGCUUCGGCCCAGAGCUGCUC (SEQ ID NO: 326) | +11G, +9G | 72.8 |
| GACACUUCGUCCCAGAGCUGCUU (SEQ ID NO: 327) | +3U, −10U | 72.7 |
| GACGCCUCGGCCCAGAGCUGCUC (SEQ ID NO: 328) | +9G, +7C | 72.5 |
| GACGCUUCUGCCCAGAGCUGCUC (SEQ ID NO: 329) | +9G, +4U | 72.4 |
| GAGAUUUCGGCCCAGAGCUGCUC (SEQ ID NO: 330) | +10G, +8U | 72.3 |
| GACACUUGGGCCCAGAGCUCCUC (SEQ ID NO: 331) | +5G, −7C | 72.3 |
| GCGACUUCGACCCAGAGCUGCUC (SEQ ID NO: 332) | +11C, +10G, +3A | 72.2 |
| GGCAGUUCGGCCCAGAGCUGCUC (SEQ ID NO: 333) | +11G, +8G | 72.0 |
| UAUACUACGGCCCAGAGCUGCUC (SEQ ID NO: 334) | +12U, +10U, +6A | 71.9 |
| GAGACUUCGACCCAGAGCUGCUC (SEQ ID NO: 335) | +10G, +3A | 71.7 |
| GACACUGCGGCGCAGAGCUGCUC (SEQ ID NO: 336) | +6G, +1G | 71.4 |
| GUCACUACGGCCCAGAGCUGCUC (SEQ ID NO: 337) | +11U, +6A | 71.4 |
| GACACAACGGCCCAGAGCUGCUC (SEQ ID NO: 338) | +7A, +6A | 71.4 |
| GACAUAUCGACCCAGAGCUGCUC (SEQ ID NO: 339) | +8U, +7A, +3A | 71.2 |
| GAUCCUUCGGCCCAGAGCUGCUC (SEQ ID NO: 340) | +10U, +9C | 71.1 |
| GAAACUUCGACCCAGAGCUGCUC (SEQ ID NO: 341) | +10A, +3A | 70.8 |
| GAAAAUUCGGUCCAGAGCUGCUC (SEQ ID NO: 342) | +10A, +8A, +2U | 70.4 |
| GGCACUUCCGCCCAGAGCUGCUC (SEQ ID NO: 343) | +11G, +4C | 70.0 |
| GACCCUUCGGCCCAGAGCUGGUU (SEQ ID NO: 344) | +9C, −8G, −10U | 70.0 |
| GAAGCUUCGGCCCAGAGCUGCUC (SEQ ID NO: 345) | +10A, +9G | 69.6 |
| GAUACUUCGACCCAGAGCUGCUC (SEQ ID NO: 346) | +10U, +3A | 69.6 |
| GGGACUUCGGCCCAGAGCUGCUC (SEQ ID NO: 347) | +11G, +10G | 69.3 |
| GACGCUUCGGCCCAGAGAUGCUC (SEQ ID NO: 348) | +9G, −5A | 69.2 |
| GACACAUCGACCCAGAGCUGCUC (SEQ ID NO: 349) | +7A, +3A | 69.2 |
| GACAGAUCGGCCCAGAGCUGCUC (SEQ ID NO: 350) | +8G, +7A | 69.1 |
| GUGACUUCGGCCCAGAGCUGCUC (SEQ ID NO: 351) | +11U, +10G | 69.0 |
| GGCACUGCGGCCCAGAGCUGCUC (SEQ ID NO: 352) | +11G, +6G | 68.8 |
| GACGCUUAGGCCCAGAGCUGCUC (SEQ ID NO: 353) | +9G, +5A | 68.8 |
| GACGCUUCGGUCCAGAGCUGCUC (SEQ ID NO: 354) | +9G, +2U | 68.4 |
| GACGCUACGGCCCAGAGCUGCUC (SEQ ID NO: 355) | +9G, +6A | 68.3 |
| GAUAUUUCGACCCAGAGCUGCUC (SEQ ID NO: 356) | +10U, +8U, +3A | 68.2 |
| GACUCUUCGACCCAGAGCUGCUC (SEQ ID NO: 357) | +9U, +3A | 68.2 |
| GACACGACGGCCCAGAGCUGCUC (SEQ ID NO: 358) | +7G, +6A | 68.1 |
| UACACUUUGACCCAGAGCUGCUC (SEQ ID NO: 359) | +12U, +5U, +3A | 68.0 |
| GACACUUCGACCCAGAGCUGUUC (SEQ ID NO: 360) | +3A, −8U | 68.0 |
| GAGACUGCGGCCCAGAGCUGCUC (SEQ ID NO: 361) | +10G, +6G | 67.9 |
| GACAUUUUGACCCAGAGCUGCUC (SEQ ID NO: 362) | +8U, +5U, +3A | 67.8 |
| GUCACUUCGACCCAGAGCUGCUC (SEQ ID NO: 363) | +11U, +3A | 67.8 |
| GGAACUUGGGCCCAGAGCUGCUC (SEQ ID NO: 364) | +11G, +10A, +5G | 67.7 |
| GACGCUUCGGCCCAGAGCUGCUC (SEQ ID NO: 365) | +9G | 67.6 |
| GACACUACGACCCAGAGCUGCUC (SEQ ID NO: 366) | +6A, +3A | 67.4 |
| GACACAGCGGCCCAGAGCUGCUC (SEQ ID NO: 367) | +7A, +6G | 67.4 |
| GACACUUCGCCCCGGAGCUGCUC (SEQ ID NO: 368) | +3C, −1G | 67.3 |
| GACACUUCGACCCAGAGCUGCUC (SEQ ID NO: 369) | +3A | 67.2 |
| GUCACUUGGGCCCAGAGCUGCUC (SEQ ID NO: 370) | +11U, +5G | 67.0 |
| GACGCUUCGGACCAGAGCUGCUC (SEQ ID NO: 371) | +9G, +2A | 67.0 |
| GACAGUUCGACCCAGAGCUGCUC (SEQ ID NO: 372) | +8G, +3A | 66.9 |
| GAGCCUUCGGCCCAGAGCUGCUC (SEQ ID NO: 373) | +10G, +9C | 66.8 |
| GACAUUUCGCCCCAGAGCUGCUC (SEQ ID NO: 374) | +8U, +3C | 66.8 |
| GACACUUGCACCCAGAGCUGCUC (SEQ ID NO: 375) | +5G, +4C, +3A | 66.7 |
| GACAGUCCGGCCCAGAGCUGCUC (SEQ ID NO: 376) | +8G, +6C | 66.7 |
| GACAUUGCGGCCCAGAGCUGCUC (SEQ ID NO: 377) | +8U, +6G | 66.7 |
| GAACCUUCGGCCCAGAGCUGCUC (SEQ ID NO: 378) | +10A, +9C | 66.7 |
| GAGACUUGGGCCCAGAGCUGCUC (SEQ ID NO: 379) | +10G, +5G | 66.7 |
| GCCACUUCGGCCCAGAGUUGCGC (SEQ ID NO: 380) | +11C, −5U, −9G | 66.7 |
| GACGCUUCGUCCCAGAGCUGCUC (SEQ ID NO: 381) | +9G, +3U | 66.3 |
| UGCACUUCGGCCCAGAGCUGCUC (SEQ ID NO: 382) | +12U, +11G | 66.1 |
| GAGACGUCGGCCCAGAGCUGCUC (SEQ ID NO: 383) | +10G, +7G | 66.1 |
| GAUACUUCUGCCCAGAGCUGCUC (SEQ ID NO: 384) | +10U, +4U | 66.0 |
| GACAUUUGGGUCCAGAGCUGCUC (SEQ ID NO: 385) | +8U, +5G, +2U | 65.8 |
| UGCACUUCGGCCCAGAGCUUCUC (SEQ ID NO: 386) | +12U, +11G, −7U | 65.8 |
| GACGCAUCGGCCCAGAGCUGCUC (SEQ ID NO: 387) | +9G, +7A | 65.8 |
| GAAACUUCGGUCCAGAGCUGCUC (SEQ ID NO: 388) | +10A, +2U | 65.8 |
| GACACUGGGGUCCAGAGCUGCUC (SEQ ID NO: 389) | +6G, +5G, +2U | 65.7 |
| GACACUGCGGCCCAGAGCUGCUU (SEQ ID NO: 390) | +6G, −10U | 65.7 |
| GUCACUUUGACCCAGAGCUGCUC (SEQ ID NO: 391) | +11U, +5U, +3A | 65.7 |
| GACACUGCGGCCCAGAGCUGCUG (SEQ ID NO: 392) | +6G, −10G | 65.6 |
| GACUCUUCGAUCCAGAGCUGCUC (SEQ ID NO: 393) | +9U, +3A, +2U | 65.4 |
| GAGACAUCGGCCCAGAGCUGCUC (SEQ ID NO: 394) | +10G, +7A | 65.4 |
| GAAACUUAGACCCAGAGCUGCUC (SEQ ID NO: 395) | +10A, +5A, +3A | 65.4 |
| GACCAGUCGGCCCAGAGCUGCUC (SEQ ID NO: 396) | +9C, +8A, +7G | 65.3 |
| GCCACUGCGGCCCAGAGCUGCUC (SEQ ID NO: 397) | +11C, +6G | 65.2 |
| GACACUCCGACCCAGAGCUGCUC (SEQ ID NO: 398) | +6C, +3A | 65.2 |
| GACACAUCGCCCCAGAGCUGCUC (SEQ ID NO: 399) | +7A, +3C | 65.2 |
| UACACUUCCGCCCAGAGCUGCUC (SEQ ID NO: 400) | +12U, +4C | 65.0 |
| GACAAUUCCGCCCAGAGGUGCUC (SEQ ID NO: 401) | +8A, +4C, −5G | 65.0 |
| GCAACUUCGGCCCAGAGCUGCUC (SEQ ID NO: 402) | +11C, +10A | 64.9 |
| GACAUUUCGACCCAGAGCUGCUU (SEQ ID NO: 403) | +8U, +3A, −10U | 64.9 |
| GACGCUACGACCCAGAGCUGCUC (SEQ ID NO: 404) | +9G, +6A, +3A | 64.9 |
| GAGACUUCGGCCCAGAGCUGCGC (SEQ ID NO: 405) | +10G, −9G | 64 9 |
| GACACUGCGACCCAGAGCUGUUC (SEQ ID NO: 406) | +6G, +3A, −8U | 64.8 |
| GAGACUUCGACCCAGAGCUGUUC (SEQ ID NO: 407) | +10G, +3A, −8U | 64.6 |
| GACGCUUCGGCCCAGAGCUACUC (SEQ ID NO: 408) | +9G, −7A | 64.6 |
| GAGACUUCGGCCCAGAGCUGCUA (SEQ ID NO: 409) | +10G, −10A | 64.5 |
| GACACAUCGGCCCAGAUCUCCUC (SEQ ID NO: 410) | +7A, −4U, −7C | 64.5 |
| GGCACAUCGGCCCAGAGCUGCUC (SEQ ID NO: 411) | +11G, +7A | 64.5 |
| GAGACUUCGCCCCAGAGCUGCUC (SEQ ID NO: 412) | +10G, +3C | 64.4 |
| GACACUACGUUCCAGAGCUGCUC (SEQ ID NO: 413) | +6A, +3U, +2U | 64.4 |
| GCCACUUCGGCCCAGAGCUCCUC (SEQ ID NO: 414) | +11C, −7C | 64.0 |
| GCCACAUCGGCCCAGAGCUGCUC (SEQ ID NO: 415) | +11C, +7A | 63.9 |
| GCCACUUCGACCCAGAGCUGGUC (SEQ ID NO: 416) | +11C, +3A, −8G | 63.9 |
| GACACUUUGACCCAGAGCUGCUC (SEQ ID NO: 417) | +5U, +3A | 63.8 |
| GGCACUUCGGCCCAGAGCUGCCC (SEQ ID NO: 418) | +11G, −9C | 63.6 |
| GACAAUUCGACCCAGAGCUGCUC (SEQ ID NO: 419) | +8A, +3A | 63.6 |
| GGCACUUCGACCCAGAGCUGCCC (SEQ ID NO: 420) | +11G, +3A, −9C | 63.5 |
| UACACUGCGGCCCAGAGCUGCUC (SEQ ID NO: 421) | +12U, +6G | 63.4 |
| GACACAUCGGCCCAGAGCUCCUC (SEQ ID NO: 422) | +7A, −7C | 63.4 |
| GACUUUUCUGCCCAGAGCUACUC (SEQ ID NO: 423) | +9U, +8U, +4U, −7A | 63.3 |
| GACCCUACGGUCCAGAGCUGCUC (SEQ ID NO: 424) | +9C, +6A, +2U | 63.3 |
| GUCAGUUCGACCCAGAGCUGCUC (SEQ ID NO: 425) | +11U, +8G, +3A | 63.2 |
| GACACUGCGGCCCAGAGCUGCUC (SEQ ID NO: 426) | +6G | 63.1 |
| GACAGUUCGUCCCAGAGCUGCUC (SEQ ID NO: 427) | +8G, +3U | 63.0 |
| GACAUGUCGGCCCAGAGCUGCUC (SEQ ID NO: 428) | +8U, +7G | 63.0 |
| GACACUUCGACCCAGAGCUGGUC (SEQ ID NO: 429) | +3A, −8G | 63.0 |
| GAGAAUUCUCCCCAGAGCUGCUC (SEQ ID NO: 430) | +10G, +8A, +4U, +3C | 63.0 |
| GCCACGUCUGCCCAGAGCUGCUC (SEQ ID NO: 431) | +11C, +7G, +4U | 63.0 |
| GACACGUCGCCCCAGAGCUGCUC (SEQ ID NO: 432) | +7G, +3C | 63.0 |
| GACAGUUCGGUCCAGAGCUGCUC (SEQ ID NO: 433) | +8G, +2U | 62.9 |
| GUCAUUUCGGCCCAGAGCUGCUC (SEQ ID NO: 434) | +11U, +8U | 62.9 |
| GACACUUCGGCCCAGAUCUCCUC (SEQ ID NO: 435) | −4U, −7C | 62.8 |
| GAGACUUCGGCCCAGAGCUGCUC (SEQ ID NO: 436) | +10G | 62.8 |
| GACACUACGGCCCAGAGCUCCUC (SEQ ID NO: 437) | +6A, −7C | 62.8 |
| GAGACUUCGGCCCAGAGCUGCUU (SEQ ID NO: 438) | +10G, −10U | 62.8 |
| GACACUUCGUUCCAGAGCUGUUC (SEQ ID NO: 439) | +3U, +2U, −8U | 62.7 |
| GAAACUUCGGCCCAGAGCUCCUC (SEQ ID NO: 440) | +10A, −7C | 62.7 |
| GACAGGUCGGCCCAGAGCUUCUC (SEQ ID NO: 441) | +8G, +7G, −7U | 62.7 |
| GACACUGCGCCCCAGAGCUGCUC (SEQ ID NO: 442) | +6G, +3C | 62.5 |
| GACUAUUCGGCCCAGAGCUGCUC (SEQ ID NO: 443) | +9U, +8A | 62.5 |
| GACCCUACGACCCAGAGCUGAUC (SEQ ID NO: 444) | +9C, +6A, +3A, −8A | 62.5 |
| GGCAUUUCGGCCCAGAGCUGCUC (SEQ ID NO: 445) | +11G, +8U | 62.5 |
| GACGGUUCCGCCCAGAGCUGCUC (SEQ ID NO: 446) | +9G, +8G, +4C | 62.5 |
| GACUCUACGGCCCAGAGCUGCUC (SEQ ID NO: 447) | +9U, +6A | 62.5 |
| GGCACUUCGGCCCAGAGCUCCUC (SEQ ID NO: 448) | +11G, −7C | 62.4 |
| GUCAGUUCGGCCCAGAGCUGCUC (SEQ ID NO: 449) | +11U, +8G | 62.4 |
| GAUACUUCGGCCCAGAGCUGCUC (SEQ ID NO: 450) | +10U | 62.4 |
| GACCCUUCGACCCAGAGCUGCUC (SEQ ID NO: 451) | +9C, +3A | 62.3 |
| GAGACUUCGACCCAGAGCUGCCC (SEQ ID NO: 452) | +10G, +3A, −9C | 62.3 |
| GAUACUUCGCCCCAGAGCUGCUC (SEQ ID NO: 453) | +10U, +3C | 62.3 |
| GAAACUUCGCCCCAGAGCUGCUC (SEQ ID NO: 454) | +10A, +3C | 62.1 |
| UACGCUUCGGCCCAGAGCUGCUC (SEQ ID NO: 455) | +12U, +9G | 62.0 |
| GACUGUUCGGCCCAGAGCUGCUC (SEQ ID NO: 456) | +9U, +8G | 61.8 |
| UACACUUCGACCCAGAGCUGCUC (SEQ ID NO: 457) | +12U, +3A | 61.8 |
| GGGACUUAGGCCCAGAGCUGCUC (SEQ ID NO: 458) | +11G, +10G, +5A | 61.8 |
| GACAGAUCGGUCCAGAGCUGCUC (SEQ ID NO: 459) | +8G, +7A, +2U | 61.8 |
| GAUACAGCGGCCCAGAGCUGCUC (SEQ ID NO: 460) | +10U, +7A, +6G | 61.8 |
| GGCACUUGGGCCCAGAGCUGCUC (SEQ ID NO: 461) | +11G, +5G | 61.6 |
| GACACUGCGACCCAAAGCUGCUC (SEQ ID NO: 462) | +6G, +3A, −2A | 61.6 |
| GACAUCUCCGCCCAGAGCUGCUC (SEQ ID NO: 463) | +8U, +7C, +4C | 61.5 |
| GACAUUUCCGCCCAGAGCUGCUC (SEQ ID NO: 464) | +8U, +4C | 61.5 |
| GACACUGCGACCCAGAGCUGCUC (SEQ ID NO: 465) | +6G, +3A | 61.4 |
| GACACUUCGACCCAGAGCUACUC (SEQ ID NO: 466) | +3A, −7A | 61.4 |
| GAUAGUCCGGCCCAGAGCUGCUC (SEQ ID NO: 467) | +10U, +8G, +6C | 61.3 |
| GACACAUCGACCCAGAGCUGGUC (SEQ ID NO: 468) | +7A, +3A, −8G | 61.3 |
| GACACGUCGGCCCAGAGUUGCUG (SEQ ID NO: 469) | +7G, −5U, −10G | 61.3 |
| GACACGACCGCCCAGAGCUGCUC (SEQ ID NO: 470) | +7G, +6A, +4C | 61.3 |
| GAGAUUCCGACCCAGAGCUGUUA (SEQ ID NO: 471) | +10G, +8U, +6C, +3A, | 61.2 |
| | −8U, −10A | |
| GCCACUUCGGCCCAGAGCUGCUC (SEQ ID NO: 472) | +11C | 61.2 |
| GAGACUACGGUCCAGAGCUCCUC (SEQ ID NO: 473) | +10G, +6A, +2U, −7C | 61.2 |
| GACGCUUCGGCCCAAAGCUGCUC (SEQ ID NO: 474) | +9G, −2A | 61.2 |
| GCCACUUCGGUCCAGAGCUGCUC (SEQ ID NO: 475) | +11C, +2U | 61.1 |
| GUCUCUUCGGCCCAGAGCUGCUC (SEQ ID NO: 476) | +11U, +9U | 61.1 |
| GCGACUUCGUCCCAGAGCUGCAC (SEQ ID NO: 477) | +11C, +10G, +3U, −9A | 61.1 |
| GGCACUUCGCCCCAGAGCUGCUC (SEQ ID NO: 478) | +11G, +3C | 61.1 |
| GUUACUUCGGCCCAGAGCUGCUC (SEQ ID NO: 479) | +11U, +10U | 61.1 |
| GGAACUUCGGCCCAGAGCUGCUC (SEQ ID NO: 480) | +11G, +10A | 61.0 |
| GACACUUCGGCCCAGAGCUCCUC (SEQ ID NO: 481) | −7C | 61.0 |

### REFERENCES

71 Buenrostro, J. D., Araya, C. L., Chircus, L. M., Layton, C. J., Chang, H. Y. Snyder, M. P., and Greenleaf, W. J. (2014). Quantitative analysis of RNA-protein interactions on a massively parallel array reveals biophysical and evolutionary landscapes. Nat Biotechnol 32, 562-568.
72 Kivioja, T., Vaharautio, A., Karlsson, K., Bonke, M., Enge, M., Linnarsson, S., and Taipale, J. (2011). Counting absolute numbers of molecules using unique molecular identifiers. Nat Methods 9, 72-74.
32 Merkle, T., Merz, S., Reautschnig, P., Blaha, A., Li, Q., Vogel., P., Wettengel, J.., Li, J. B., and Stafforst, T. (2019) Precise RNA editing by recruiting endogenous ADARs with antisense oligonucleotides. Nat Biotechnol 37, 133-138.
49 Wong, S. K., Sato, S., and Lazinski, D. W. (2001). Substrate recognition by ADAR1 and ADAR2. RNA 7, 846-858.

1.-23. (canceled)

24. A high-throughput screening method for selecting guide RNAs for use in site-directed RNA editing, the method comprising:
a. generating a plurality of fusion constructs, each fusion construct comprising a target sequence and a guide RNA sequence, wherein the guide RNA sequence comprises an antisense domain that is substantially complementary or perfectly complementary to the target sequence;
b. expressing each of the plurality of fusion constructs in a distinct population of cells; and
c. determining whether a fusion construct induces one or more modifications in nucleic acid isolated from the population of cells expressing the fusion construct.

25. The method of claim 24, wherein the cells express endogenous adenosine deaminases acting on RNA (ADARs) and/or at least one engineered ADAR fusion protein.

26. The method of claim 24, wherein the guide RNA sequence further comprises a recruitment domain that recruits endogenous adenosine deaminases acting on RNA (ADARs) and/or engineered ADAR fusion proteins.

27. The method of claim 26, wherein the recruitment domain comprises a first strand and a second strand that are substantially complementary or perfectly complementary to each other.

28. The method of claim 24, wherein the fusion construct further comprises a loop sequence, such that the construct forms a stem loop secondary structure.

29. The method of claim 28, wherein the loop sequence comprises 3-50 nucleotides.

30. The method of claim 29, wherein the loop sequence comprises 5 nucleotides.

31. The method of claim 30, wherein the loop sequence comprises a nucleotide sequence set forth in Table 1.

32. The method of claim 28, wherein the antisense domain and the target sequence are linked by the loop sequence.

33. The method of claim 28, wherein the first strand and the second strand of the recruitment domain are linked by the loop sequence.

34. The method of claim 24, wherein the guide RNA sequence comprises one or more mutations in the antisense domain that disrupt base pairing between the antisense domain and the target sequence in at least one nucleotide location.

35. The method of claim 27, wherein the guide RNA sequence comprises one or more mutations in the first strand and/or the second strand of the recruitment domain that disrupt base pairing between the first strand and the second strand in at least one nucleotide location.

36. (canceled)

37. (canceled)

38. The method of claim 35, wherein the first strand comprises a nucleotide sequence set forth in Table 2.

39. (canceled)

40. (canceled)

41. (canceled)

42. The method of claim 24, wherein the target sequence is derived from a gene for which site-directed A-to-I RNA editing is desired.

43. The method of claim 42, wherein the gene comprises a point mutation, wherein the point mutation is a G to A point mutation, a T to A point mutation, or C to A point mutation.

44. The method of claim 43, wherein the point mutation is associated with development of a disease or condition in a subject expressing the gene.

45. The method of claim wherein the point mutation is present in the target sequence.

46. The method of claim 45, wherein determining whether a fusion construct induces one or more modifications in nucleic acid isolated from the population of cells expressing the fusion construct comprises sequencing the isolated nucleic acid.

47. The method of claim 46, wherein the isolated nucleic acid comprises RNA.

48. The method of claim 46, wherein the one or more modifications in nucleic acid isolated from the population of cells comprises a correction of the point mutation initially present in the target sequence.

49. The method of claim 48, wherein correction of the point mutation indicates that the guide RNA sequence effectively induces site-directed RNA editing.

50. (canceled)

51. (canceled)

52. The method of claim 24, wherein the antisense domain comprises a sequence set forth in Table 5 or Table 6.

53. The method of claim 24, wherein the method identifies one or more optimized features of the guide RNA sequence that enable the guide RN sequence to induce one or more modifications in nucleic acid isolated from the population of cells expressing the fusion construct.

54. The method of claim 53, wherein the optimized features are selected from the antisense domain, the loop sequence, and the recruitment domain, if present in the guide RNA.

55.-71. (canceled)


SEQUENCE LISTING (481 sequences)

SEQ ID NO: 1 - RNA, 73 nt, Artificial sequence
  Synthetic
ggugucgagaagaggagaacaauaugccaaauguuguucuccucuccucgacaccuucggcccagagcugcuc

SEQ ID NO: 2 - RNA, 73 nt, Artificial sequence
  Synthetic
ggugucgagaagaugagaacaauaugccaaauguuguucucgucuccucgacaccuucggcccagagcugcuc

SEQ ID NO: 3 - RNA, 73 nt, Artificial sequence
  Synthetic
ggugucgagaagaggagaacaauaugccaaauguuguucucgucuucucgacaccuucggcccagagcugcuc

SEQ ID NO: 4 - RNA, 73 nt, Artificial sequence
  Synthetic
ggugucgagaagaggagaacaauaucuuucauguuguucucgucuccucgacaccuucggcccagagcugcuc

SEQ ID NO: 5 - RNA, 73 nt, Artificial sequence
  Synthetic
ggugucgagaagaggagaacaauauguuuuauguuguucucgucuccucgacaccuucggcccagagcugcuc

SEQ ID NO: 6 - RNA, 73 nt, Artificial sequence
  Synthetic
ggugucgagaagaggagaacaauaugauugauguuguucucgucuccucgacaccuucggcccagagcugcuc

SEQ ID NO: 7 - RNA, 73 nt, Artificial sequence
  Synthetic
ggugucgagaagaggagaacaauauuuuuuauguuguucucgucuccucgacaccuucggcccagagcugcuc

SEQ ID NO: 8 - RNA, 73 nt, Artificial sequence
  Synthetic
ggugucgagaagaggagaacaauaugguugauguuguucucgucuccucgacaccuucggcccagagcugcuc

SEQ ID NO: 9 - RNA, 73 nt, Artificial sequence
  Synthetic
ggugucgagaagaggagaacaauauuuuucauguuguucucgucuccucgacaccuucggcccagagcugcuc

SEQ ID NO: 10 - RNA, 73 nt, Artificial sequence
  Synthetic
ggugucgagaagaggagaacaauaugauuuauguuguucucgucuccucgacaccuucggcccagagcugcuc

SEQ ID NO: 11 - RNA, 33 nt, Homo sapiens
gaugaggagcagcucuaggccgaagugucgcag

SEQ ID NO: 12 - protein, 11 aa, Homo sapiens
  misc_feature (6)..(6): Xaa can be any naturally occurring amino acid
DEEQLXAEVSQ

SEQ ID NO: 13 - RNA, 91 nt, Artificial sequence
  Synthetic
  misc_feature (74)..(91): n is a, c, g, or u
gagcagcucuaggccgaaggugucgagaagaggagaacaauaugccaaauguuguucucgucuccucgacaccnnnnnnnnnnnnnnnnnn

SEQ ID NO: 14 - RNA, 91 nt, Artificial sequence
  Synthetic
gagcagcucuaggccgaaggugucgagaagaggagaacaauaugccaaauguuguucucgucuccucgacaccuucggcccagagcugcuc

SEQ ID NO: 15 - RNA, 73 nt, Artificial sequence
  Synthetic
gagcagcucuaggccgaaggugucgagaagaggagaacaauaugccaaauguuguucucgucuccucgacacc

SEQ ID NO: 16 - RNA, 103 nt, Artificial sequence
  Synthetic
gagcagcucuaggccgaaggugucgagaagaggagaacaauaugccaaauguuguucucgucuccucgacaccuucggcccagagcugcuccucauccagcag

SEQ ID NO: 17 - RNA, 103 nt, Artificial sequence
  Synthetic
gagcagcucuaggccgaaggugucgagaagaggagaacaauaugccaaauguuguucucgucuccucgacaccggccugcgacacuucggcccagagcugcuc

SEQ ID NO: 18 - RNA, 91 nt, Artificial sequence
  Synthetic
  misc_feature (19)..(73): n is a, c, g, or u
gagcagcucuaggccgaannnnnnnnnnnnnnnnnnnnnnnnnnnnnnnnnnnnnnnnnnnnnnnnnnnnnnnuucggcccagagcugcuc

SEQ ID NO: 19 - RNA, 91 nt, Artificial sequence
  Synthetic
  misc_feature (19)..(43): n is a, c, g, or u
  misc_feature (49)..(73): n is a, c, g, or u
gagcagcucuaggccgaannnnnnnnnnnnnnnnnnnnnnnnngccaannnnnnnnnnnnnnnnnnnnnnnnnuucggcccagagcugcuc

SEQ ID NO: 20 - RNA, 107 nt, Artificial sequence
  Synthetic
gagcagcucuaggccgaagcgcgcgcggugucgagaagaggagaacaauaugccaaauguuguucucgucuccucgacaccgcgcgcgcuucggcccagagcugcuc

SEQ ID NO: 21 - RNA, 107 nt, Artificial sequence
  Synthetic
gagcagcucuaggccgaaggugucgagaagaggagaacaauaugcgcgcgcgccaagcgcgcgcauguuguucucgucuccucgacaccuucggcccagagcugcuc

SEQ ID NO: 22 - RNA, 79 nt, Artificial sequence
  Synthetic
gagcagcucuaggccgaagugucgcaggccgggaccggccaacggucccggccggcgccacuucggcccagagcugcuc

SEQ ID NO: 23 - RNA, 117 nt, Artificial sequence
  Synthetic
augaggagcagcucuaggccgaagugucgcaggugucgagaagaggagaacaauaugccaaauguuguucucgucuccucgacaccugcgacacuucggcccagagcugcuccucau

SEQ ID NO: 24 - DNA, 133 bp, Artificial sequence
  Synthetic
tcgcccttgctcaccatatcgataagagcagctctgggccgaaggtgtcgaggagacgagaacaacatttggcatattgttctctcttctcgacaccttcggcctagagctgctcgctagccttgtacagctc

SEQ ID NO: 25 - DNA, 134 bp, Artificial sequence
  Synthetic
agcgggaacgactggtatagctattctcgtcgagacccggcttccacagctcctctgctcttgttgtaaaccgtataacaagaggagaagagctgtggaagccggatctcgacgagcgatcggaacatgtcgag

SEQ ID NO: 26 - protein, 44 aa, Artificial sequence
  Synthetic
EGKSVMDILAARPRLHRPSSFLMQCITRRKEVGEALQESAKYLE

SEQ ID NO: 27 - protein, 6 aa, Artificial sequence
  Synthetic
EFKSVM

SEQ ID NO: 28 - DNA, 194 bp, Artificial sequence
  Synthetic
tcgcccttgctcaccatatcgatggggcggtgggcgctggccaggacgcccaccgtgtggttgctgtccaggacggtcccggcctgcgacacttcggcctagagctgctcctcatccagcagcgccagcagccccatggccgtagagcaccggcttgcgcaacagctgcacgtggctagccttgtacagctcgt

SEQ ID NO: 29 - DNA, 193 bp, Artificial sequence
  Synthetic
agcgggaacgagtggtatagctaccccgccacccgcgaccggtcctgcgggtggcacaccaacgacaggtcctgccagggccggacgctgtgaagccggatctcgacgaggagtaggtcgtcgcggtcgtcggggtaccggcactcgtggccgaacgcgttgtcgacgtgcaccgatcggaacatgtcgagca

SEQ ID NO: 30 - protein, 63 aa, Artificial sequence
  Synthetic
EGKSVMDIPRHASALVGVTHNSDLVTGAQSVEALQEEDLLALGMATLVPKRLLQVHSAKYLED

SEQ ID NO: 31 - DNA, 163 bp, Artificial sequence
  Synthetic
ctgcaataaacaagttaacaacaacgattaattaaatgaggagcagctctgggccgaagtgtcgcaggtcctgaggagacgagaacaacatttggcatattgttctcctcttctcaggaccattcgaatttcctaggttcagggggaggtgtgggaggttttt

SEQ ID NO: 32 - DNA, 163 bp, Artificial sequence
  Synthetic
gacgttatttgttcaattgttgttgctaattaatttactcctcgtcgagacccggcttcacagcgtccaggactcctctgctcttgttgtaaaccgtataacaagaggagaagagtcctggtaagcttaaaggatccaagtccccctccacaccctccaaaaa

SEQ ID NO: 33 - DNA, 168 bp, Artificial sequence
  Synthetic
taaagcaagtaaaacctctacaaatgtggtatggctgattatgatcctctggagatgcggccgcttacttgtacttgtacagctcgtccatgccgagagtgatcccggcggcggtcacgaactccagcaggaccatgtgtcgcgcttctcgttggggtctttgctcag

SEQ ID NO: 34 - DNA, 163 bp, Artificial sequence
  Synthetic
atttcgttcattttggagatgtttacaccataccgactaatactaggagacctctacgccggcgaatgaacatgtcgagcaggtacggctctcactagggccgccgccagtgcttgaggtcgtcctggtacactagcgcgaagagcaaccccagaaacgagtc

SEQ ID NO: 35 - protein, 32 aa, Artificial sequence
  Synthetic
KYLEDMGLTIGAATVFELLVMHDRKENPDKSL

SEQ ID NO: 36 - DNA, 89 bp, Artificial sequence
  Synthetic
caccaccccggtgaacagctcctcgcccttgctcaccatatcgataagagcagctctgggccgaaggtgtcgaggagacgagaacaaca

SEQ ID NO: 37 - DNA, 89 bp, Artificial sequence
  Synthetic
gtggtggggccacttgtcgaggagcgggaacgagtggtatagctattctcgtcgagacccggcttccacagctcctctgctcttgttgt

SEQ ID NO: 38 - protein, 26 aa, Artificial sequence
  Synthetic
FLEEGKSVMDILAARPRLHRPSSFLM

SEQ ID NO: 39 - protein, 13 aa, Artificial sequence
  Synthetic
VVGTFLEEGKSVM

SEQ ID NO: 40 - DNA, 89 bp, Artificial sequence
  Synthetic
tttggcatattgttctcctcttctcgacaccttcggcctagagctgctcgctagccttgtacagctcgtccatgccgccggtggagtgg

SEQ ID NO: 41 - DNA, 89 bp, Artificial sequence
  Synthetic
aaaccgtataacaagaggagaagagctgtggaagccggatctcgacgagcgatcggaacatgtcgagcaggtacggcggccacctcacc

SEQ ID NO: 42 - protein, 28 aa, Artificial sequence
  Synthetic
QCITRRKEVGEALQESAKYLEDMGGTSH

SEQ ID NO: 43 - DNA, 199 bp, Artificial sequence
  Synthetic
aatgatacggcgaccaccgagatctacactctttccctacagacgctcttccgatcttcgtggaacagtacgaacgcgccgagggccgccactccaccggcggcatggacgagctgtacaaggctagcgagcagctctaggccgaaggtgtcgagaagaggagaacaatatgccaaatgttgttctcgtctcctcgaca

SEQ ID NO: 44 - DNA, 199 bp, Artificial sequence
  Synthetic
ttactatgccgctggtggctctagatgtgagaaagggatgtgctgcgagaaggctagaagcaccttgtcatgcttgcgcggctcccggcggtgaggtggccgccgtacctgctcgacatgttccgatcgctcgtcgagatccggcttccacagctcttctcctcttgttatacggtttacaacaagagcgaggagctgt

SEQ ID NO: 45 - DNA, 199 bp, Artificial sequence
  Synthetic
  misc_feature (121)..(135): n is a, c, g, or t
ccttcggcccagagctgctcttatcgatatggtgagcaagggcgaggagctgttcaccggggtggtgcccatcctggtcgagctggacggcgacgtaaacggccacaagttcagcgtgtcnnnnnnnnnnnnnnnagatcggaagagcacacgtctgaactccagtcacatgtcaatctcgtatgccgtcttctgcttg

SEQ ID NO: 46 - DNA, 197 bp, Artificial sequence
  Synthetic
  misc_feature (119)..(133): n is a, c, g, or t
ggaagccgggtctcgacgagaatgctataccactcgttcccgctcttcgacaagtggccccaccacgggtaggaccagctcgacctgccgctgcatttgccggtgttcaagtcgacagnnnnnnnnnnnnnnntctagccttctcgtgtgcagacttgaggtcagtgtacagttagagcatacggcagaagacgaac

SEQ ID NO: 47 - RNA, 91 nt, Artificial sequence; identical to SEQ ID NO: 14
  Synthetic

SEQ ID NO: 48 - RNA, 92 nt, Artificial sequence
  Synthetic
gagcagcucuaggccgaaggugucgagaagaggagaacaauaugccaaauguuguucucgucuccucgacaccaauugccccagagcuccuc

SEQ ID NO: 49 - RNA, 91 nt, Artificial sequence
  Synthetic
gagcagcucuaggccgaaggugucgagaagaggagaacaauaugccaaauguuguucucgucuccucgacaccuucgacccagagcuccuc

SEQ ID NO: 50 - RNA, 91 nt, Artificial sequence
  Synthetic
gagcagcucuaggccgaaggugucgagaagaggagaacaauaugccaaauguuguucucgucuccucgacaccuucgacccagagcugcuc

SEQ ID NO: 51 - RNA, 91 nt, Artificial sequence
  Synthetic
gagcagcucuaggccgaaggugucgagaagaggagaacaauaugccaaauguuguucucgucuccucgacaccuacggcccagagcuccuc

SEQ ID NO: 52 - RNA, 91 nt, Artificial sequence
  Synthetic
gagcagcucuaggccgaaggugucgagaagaggagaacaauaugccaaauguuguucuccucuccucgacaccuucggcccagagcugcuc

SEQ ID NO: 53 - RNA, 91 nt, Artificial sequence
  Synthetic
gagcagcucuaggccgaaggugucgagaagaggagaacaauaugccaaauguuguucucuucuccucgacaccuucggcccagagcugcuc

SEQ ID NO: 54 - RNA, 91 nt, Artificial sequence
  Synthetic
gagcagcucuaggccgaaggugucgagaauaggagaacaauaugccaaauguuguucucgucuccucgacaccuucggcccagagcugcuc

SEQ ID NO: 55 - RNA, 92 nt, Artificial sequence
  Synthetic
gagcagcucuaggccgaaggugugcgagaagaggagaacaauaugccaaauguuguucucgucuccucgacgccuucgacccagagcugcuc

SEQ ID NO: 56 - RNA, 91 nt, Artificial sequence
  Synthetic
gagcagcucuaggccgaaggugucgagaagaggagaacaauaugccaaauguuguucucgucuccucgacgccuucggcccagagcuccuc

SEQ ID NO: 57 - RNA, 91 nt, Artificial sequence
  Synthetic
gagcagcucuaggccgaaggugucgagaagaggagaacaauaugccaaauguuguucucgucuccucgacgccuucgacccagagcuccuc

SEQ ID NO: 58 - RNA, 5 nt, Artificial sequence
  Synthetic
ggguu

SEQ ID NO: 59 - RNA, 5 nt, Artificial sequence
  Synthetic
agguu

SEQ ID NO: 60 - RNA, 5 nt, Artificial sequence
  Synthetic
guugu

SEQ ID NO: 61 - RNA, 5 nt, Artificial sequence
  Synthetic
aauuu

SEQ ID NO: 62 - RNA, 5 nt, Artificial sequence
  Synthetic
uuugu

SEQ ID NO: 63 - RNA, 5 nt, Artificial sequence
  Synthetic
ggaac

SEQ ID NO: 64 - RNA, 5 nt, Artificial sequence
  Synthetic
cuguu

SEQ ID NO: 65 - RNA, 5 nt, Artificial sequence
  Synthetic
auuuu

SEQ ID NO: 66 - RNA, 5 nt, Artificial sequence
  Synthetic
auuua

SEQ ID NO: 67 - RNA, 5 nt, Artificial sequence
  Synthetic
uugag

SEQ ID NO: 68 - RNA, 5 nt, Artificial sequence
  Synthetic
aguuc

SEQ ID NO: 69 - RNA, 5 nt, Artificial sequence
  Synthetic
guugg

SEQ ID NO: 70 - RNA, 5 nt, Artificial sequence
  Synthetic
caauu

SEQ ID NO: 71 - RNA, 5 nt, Artificial sequence
  Synthetic
cuugg

SEQ ID NO: 72 - RNA, 5 nt, Artificial sequence
  Synthetic
uacuu

SEQ ID NO: 73 - RNA, 5 nt, Artificial sequence
  Synthetic
aauuc

SEQ ID NO: 74 - RNA, 5 nt, Artificial sequence
  Synthetic
uugau

SEQ ID NO: 75 - RNA, 5 nt, Artificial sequence
  Synthetic
gauuc

SEQ ID NO: 76 - RNA, 5 nt, Artificial sequence
  Synthetic
uauuc

SEQ ID NO: 77 - RNA, 5 nt, Artificial sequence
  Synthetic
auuca

SEQ ID NO: 78 - RNA, 5 nt, Artificial sequence
  Synthetic
guucg

SEQ ID NO: 79 - RNA, 5 nt, Artificial sequence
  Synthetic
uuucu

SEQ ID NO: 80 - RNA, 5 nt, Artificial sequence
  Synthetic
uucuu

SEQ ID NO: 81 - RNA, 5 nt, Artificial sequence
  Synthetic
caguu

SEQ ID NO: 82 - RNA, 5 nt, Artificial sequence
  Synthetic
uauug

SEQ ID NO: 83 - RNA, 5 nt, Artificial sequence
  Synthetic
uguua

SEQ ID NO: 84 - RNA, 5 nt, Artificial sequence
  Synthetic
uuauu

SEQ ID NO: 85 - RNA, 5 nt, Artificial sequence
  Synthetic
uuuaa

SEQ ID NO: 86 - RNA, 5 nt, Artificial sequence
  Synthetic
ugguu

SEQ ID NO: 87 - RNA, 5 nt, Artificial sequence
  Synthetic
aaguu

SEQ ID NO: 88 - RNA, 5 nt, Artificial sequence
  Synthetic
guccc

SEQ ID NO: 89 - RNA, 5 nt, Artificial sequence
  Synthetic
gacuu

SEQ ID NO: 90 - RNA, 5 nt, Artificial sequence
  Synthetic
uuucg

SEQ ID NO: 91 - RNA, 5 nt, Artificial sequence
  Synthetic
uuugg

SEQ ID NO: 92 - RNA, 5 nt, Artificial sequence
  Synthetic
gaguu

SEQ ID NO: 93 - RNA, 5 nt, Artificial sequence
  Synthetic
ccuua

SEQ ID NO: 94 - RNA, 5 nt, Artificial sequence
  Synthetic
guuac

SEQ ID NO: 95 - RNA, 5 nt, Artificial sequence
  Synthetic
auuga

SEQ ID NO: 96 - RNA, 5 nt, Artificial sequence
  Synthetic
gcuua

SEQ ID NO: 97 - RNA, 5 nt, Artificial sequence
  Synthetic
agcuu

SEQ ID NO: 98 - RNA, 5 nt, Artificial sequence
  Synthetic
aguua

SEQ ID NO: 99 - RNA, 5 nt, Artificial sequence
  Synthetic
uccau

SEQ ID NO: 100 - RNA, 5 nt, Artificial sequence
  Synthetic
ggucg

SEQ ID NO: 101 - RNA, 5 nt, Artificial sequence
  Synthetic
ccuuc

SEQ ID NO: 102 - RNA, 5 nt, Artificial sequence
  Synthetic
ccguu

SEQ ID NO: 103 - RNA, 5 nt, Artificial sequence
  Synthetic
uuugc

SEQ ID NO: 104 - RNA, 5 nt, Artificial sequence
  Synthetic
uugaa

SEQ ID NO: 105 - RNA, 5 nt, Artificial sequence
  Synthetic
ucauu

SEQ ID NO: 106 - RNA, 5 nt, Artificial sequence
  Synthetic
cuuga

SEQ ID NO: 107 - RNA, 5 nt, Artificial sequence
  Synthetic
aaauu

SEQ ID NO: 108 - RNA, 25 nt, Artificial sequence
  Synthetic
gguggcuagaagaggagagcaaaau

SEQ ID NO: 109 - RNA, 25 nt, Artificial sequence
  Synthetic
ggagucgagaugaggagggccauau

SEQ ID NO: 110 - RNA, 25 nt, Artificial sequence
  Synthetic
gaugucgagaagaggagaacuauau

SEQ ID NO: 111 - RNA, 25 nt, Artificial sequence
  Synthetic
gguguuaagaagaggagaacaauuu

SEQ ID NO: 112 - RNA, 25 nt, Artificial sequence
  Synthetic
ggugacgagaagaugagaacaauag

SEQ ID NO: 113 - RNA, 25 nt, Artificial sequence
  Synthetic
gguguggagaagaggagaacacuau

SEQ ID NO: 114 - RNA, 25 nt, Artificial sequence
  Synthetic
gaugucuagaagaggagaacacuau

SEQ ID NO: 115 - RNA, 25 nt, Artificial sequence
  Synthetic
ggucuggagaacaggagaacaauau

SEQ ID NO: 116 - RNA, 25 nt, Artificial sequence
  Synthetic
ggagucgagcagaggaaaaccauau

SEQ ID NO: 117 - RNA, 25 nt, Artificial sequence
  Synthetic
gaugucuagaagaggagaacaaugu

SEQ ID NO: 118 - RNA, 25 nt, Artificial sequence
  Synthetic
gguuucgggaagaggagaacaauag

SEQ ID NO: 119 - RNA, 25 nt, Artificial sequence
  Synthetic
ggugccgagaagacgagaacaauaa

SEQ ID NO: 120 - RNA, 25 nt, Artificial sequence
  Synthetic
gaugucgagaagaugagaacaauau

SEQ ID NO: 121 - RNA, 25 nt, Artificial sequence
  Synthetic
ggugucgagaagccgagaacaauau

SEQ ID NO: 122 - RNA, 25 nt, Artificial sequence
  Synthetic
gaugucgagaaguggagaacaauau

SEQ ID NO: 123 - RNA, 25 nt, Artificial sequence
  Synthetic
gguaucaagaagaugagaacaauaa

SEQ ID NO: 124 - RNA, 25 nt, Artificial sequence
  Synthetic
ggugacgagaaguugagaacaauau

SEQ ID NO: 125 - RNA, 25 nt, Artificial sequence
  Synthetic
ggcgucgagaagaggagaacaaggu

SEQ ID NO: 126 - RNA, 25 nt, Artificial sequence
  Synthetic
gguguugagaagacgagaacaauau

SEQ ID NO: 127 - RNA, 25 nt, Artificial sequence
  Synthetic
ggugucgagaaguugagaacaaucu

SEQ ID NO: 128 - RNA, 25 nt, Artificial sequence
  Synthetic
ggugucgcgaagcggagaacauuau

SEQ ID NO: 129 - RNA, 25 nt, Artificial sequence
  Synthetic
guugucgagaaggggagaacaauau

SEQ ID NO: 130 - RNA, 25 nt, Artificial sequence
  Synthetic
ggugucgagaagaugagaacaauau

SEQ ID NO: 131 - RNA, 25 nt, Artificial sequence
  Synthetic
ggugucgagaauacgagaaaaauug

SEQ ID NO: 132 - RNA, 25 nt, Artificial sequence
  Synthetic
guuuucgacaagaggcaaacauugu

SEQ ID NO: 133 - RNA, 25 nt, Artificial sequence
  Synthetic
gguguggagaagacgagagcaauuu

SEQ ID NO: 134 - RNA, 25 nt, Artificial sequence
  Synthetic
ggugucgagaagaggagaacaauag

SEQ ID NO: 135 - RNA, 25 nt, Artificial sequence
  Synthetic
gaugucgagaagaggagaacaauuu

SEQ ID NO: 136 - RNA, 25 nt, Artificial sequence
  Synthetic
ggugucgagaagaggagaacaacau

SEQ ID NO: 137 - RNA, 25 nt, Artificial sequence
  Synthetic
ggugucgagaagaggagaauaacau

SEQ ID NO: 138 - RNA, 25 nt, Artificial sequence
  Synthetic
gaugucgagaauaugagaacaauau

SEQ ID NO: 139 - RNA, 25 nt, Artificial sequence
  Synthetic
gaugucgagaagaggagaacaauau

SEQ ID NO: 140 - RNA, 25 nt, Artificial sequence
  Synthetic
ggugaugagaagaggagaacaauau

SEQ ID NO: 141 - RNA, 25 nt, Artificial sequence
  Synthetic
gaugucgagaagaggagaacaauaa

SEQ ID NO: 142 - RNA, 25 nt, Artificial sequence
  Synthetic
gaugucuagaagaugagaacaauau

SEQ ID NO: 143 - RNA, 25 nt, Artificial sequence
  Synthetic
gaugucuagaagaugagaacaauau

SEQ ID NO: 144 - RNA, 25 nt, Artificial sequence
  Synthetic
auguugugcucgucuccucgacgcc

SEQ ID NO: 145 - RNA, 25 nt, Artificial sequence
  Synthetic
auguucuucucgucugcuggacacu

SEQ ID NO: 146 - RNA, 25 nt, Artificial sequence
  Synthetic
auguuguucucgcccccucggcacc

SEQ ID NO: 147 - RNA, 25 nt, Artificial sequence
  Synthetic
auguuguucuccucuccucgacacc

SEQ ID NO: 148 - RNA, 25 nt, Artificial sequence
  Synthetic
agguuguucucgucuccucaacacc

SEQ ID NO: 149 - RNA, 25 nt, Artificial sequence
  Synthetic
guguuguucucgucuccgcgacaac

SEQ ID NO: 150 - RNA, 25 nt, Artificial sequence
  Synthetic
auguuguucuccucuucucgacacc

SEQ ID NO: 151 - RNA, 25 nt, Artificial sequence
  Synthetic
auguuguucuccucucgucgacauc

SEQ ID NO: 152 - RNA, 25 nt, Artificial sequence
  Synthetic
augcuguucuccucuccucgacacc

SEQ ID NO: 153 - RNA, 25 nt, Artificial sequence
  Synthetic
auguuguucuccucucuucgacacc

SEQ ID NO: 154 - RNA, 25 nt, Artificial sequence
  Synthetic
auauugcucucgucuccuggucacc

SEQ ID NO: 155 - RNA, 25 nt, Artificial sequence
  Synthetic
auguuguucuucucuccucgacacc

SEQ ID NO: 156 - RNA, 25 nt, Artificial sequence
  Synthetic
guguuguucucgucuccucaacacc

SEQ ID NO: 157 - RNA, 25 nt, Artificial sequence
  Synthetic
aacuuguucucgucuccuccacacc

SEQ ID NO: 158 - RNA, 25 nt, Artificial sequence
  Synthetic
augauguucucgucugcucgacccc

SEQ ID NO: 159 - RNA, 25 nt, Artificial sequence
  Synthetic
guguuguucucgucuccucgacagc

SEQ ID NO: 160 - RNA, 25 nt, Artificial sequence
  Synthetic
auguuguucucuucuccucgacacc

SEQ ID NO: 161 - RNA, 25 nt, Artificial sequence
  Synthetic
auguuguucucuucuccucgacacc

SEQ ID NO: 162 - RNA, 25 nt, Artificial sequence
  Synthetic
acguuguucucuucuccucgacacc

SEQ ID NO: 163 - RNA, 25 nt, Artificial sequence
  Synthetic
auguuguucucaucuccucgacagc

SEQ ID NO: 164 - RNA, 25 nt, Artificial sequence
  Synthetic
auguuguccuccucuccucgacacc

SEQ ID NO: 165 - RNA, 25 nt, Artificial sequence
  Synthetic
augcuguucucgucuccucggcacc

SEQ ID NO: 166 - RNA, 25 nt, Artificial sequence
  Synthetic
aucuuguucucgucuccucgacauc

SEQ ID NO: 167 - RNA, 25 nt, Artificial sequence
  Synthetic
agguuguucucgucuccucgacauc

SEQ ID NO: 168 - RNA, 25 nt, Artificial sequence
  Synthetic
agguuguucucgucuccucgacauc

SEQ ID NO: 169 - RNA, 25 nt, Artificial sequence
  Synthetic
guguuguucucgucuccucgauacc

SEQ ID NO: 170 - RNA, 25 nt, Artificial sequence
  Synthetic
uuguuguccucgucuccucgacacc

SEQ ID NO: 171 - RNA, 25 nt, Artificial sequence
  Synthetic
cuguuguucucgucuccucgacacc

SEQ ID NO: 172 - RNA, 25 nt, Artificial sequence
  Synthetic
guguuguucucgucuccucgagacc

SEQ ID NO: 173 - RNA, 25 nt, Artificial sequence
  Synthetic
guguuguucucgucuccucgagacc

SEQ ID NO: 174 - RNA, 25 nt, Artificial sequence
  Synthetic
guguuguucucgucugcucgacacc

SEQ ID NO: 175 - RNA, 25 nt, Artificial sequence
  Synthetic
auguuguucucuucuccucaacacc

SEQ ID NO: 176 - RNA, 25 nt, Artificial sequence
  Synthetic
uuguuguucucgccuccucgagacc

SEQ ID NO: 177 - RNA, 25 nt, Artificial sequence
  Synthetic
uuguuguucucgccuccucgagacc

SEQ ID NO: 178 - RNA, 25 nt, Artificial sequence
  Synthetic
auguuguucucuucuccucgagacc

SEQ ID NO: 179 - RNA, 25 nt, Artificial sequence
  Synthetic
cuauuguucucgucuccucgacacc

SEQ ID NO: 180 - RNA, 25 nt, Artificial sequence
  Synthetic
auguuguucucuucuccucggccuc

SEQ ID NO: 181 - RNA, 25 nt, Artificial sequence
  Synthetic
auguuguucuccuccccucgacacc

SEQ ID NO: 182 - RNA, 25 nt, Artificial sequence
  Synthetic
auguuguucucaccuucucgacacc

SEQ ID NO: 183 - RNA, 25 nt, Artificial sequence
  Synthetic
agguuguucucgucuccuugucacc

SEQ ID NO: 184 - RNA, 25 nt, Artificial sequence
  Synthetic
auguuguucuccucuccuugacacc

SEQ ID NO: 185 - RNA, 25 nt, Artificial sequence
  Synthetic
agguuguucucgucuccucgacacc

SEQ ID NO: 186 - RNA, 25 nt, Artificial sequence
  Synthetic
auguuguucucgucuccucgacggc

SEQ ID NO: 187 - RNA, 25 nt, Artificial sequence
  Synthetic
cuguuguucucuucuccucgacacc

SEQ ID NO: 188 - RNA, 25 nt, Artificial sequence
  Synthetic
auguuguucucgucuccuggccacc

SEQ ID NO: 189 - RNA, 25 nt, Artificial sequence
  Synthetic
auguuguucuccucuccacgaaucc

SEQ ID NO: 190 - RNA, 25 nt, Artificial sequence
  Synthetic
guguugaucucuucuccucgacacc

SEQ ID NO: 191 - RNA, 25 nt, Artificial sequence
  Synthetic
uuguuguucucgucuccucgagacc

SEQ ID NO: 192 - RNA, 25 nt, Artificial sequence
  Synthetic
auguuguucucgucuccucgaaccc

SEQ ID NO: 193 - RNA, 25 nt, Artificial sequence
  Synthetic
acguuguucucgucuccucgaaauc

SEQ ID NO: 194 - RNA, 25 nt, Artificial sequence
  Synthetic
auguuguucucgucuccuggacucc

SEQ ID NO: 195 - RNA, 18 nt, Artificial sequence
  Synthetic
auugccccagagcugcuc

SEQ ID NO: 196 - RNA, 18 nt, Artificial sequence
  Synthetic
gucgacccagagcuccuc

SEQ ID NO: 197 - RNA, 18 nt, Artificial sequence
  Synthetic
uucgacccagagcuccuc

SEQ ID NO: 198 - RNA, 18 nt, Artificial sequence
  Synthetic
uucgacccagagcuccuc

SEQ ID NO: 199 - RNA, 18 nt, Artificial sequence
  Synthetic
uacggcccagagcuccuc

SEQ ID NO: 200 - RNA, 18 nt, Artificial sequence
  Synthetic
uucgacccaaagcugcuc

SEQ ID NO: 201 - RNA, 18 nt, Artificial sequence
  Synthetic
uuagacccagagcuucuc

SEQ ID NO: 202 - RNA, 23 nt, Artificial sequence
  Synthetic
gucgacccagagcugcucaucau

SEQ ID NO: 203 - RNA, 23 nt, Artificial sequence
  Synthetic
uucggugcagagcugcuccucau

SEQ ID NO: 204 - RNA, 23 nt, Artificial sequence
  Synthetic
uucgccccagagcugcuccccaa

SEQ ID NO: 205 - RNA, 23 nt, Artificial sequence
  Synthetic
uucgacccagagcugcuccugaa

SEQ ID NO: 206 - RNA, 23 nt, Artificial sequence
  Synthetic
uucgcccccgagcagcacgucau

SEQ ID NO: 207 - RNA, 23 nt, Artificial sequence
  Synthetic
uacggcccagagcuccuccucgu

SEQ ID NO: 208 - RNA, 23 nt, Artificial sequence
  Synthetic
ugaggcccagagcuccuccucac

SEQ ID NO: 209 - RNA, 23 nt, Artificial sequence
  Synthetic
uucggcccagaguugcuccccau

SEQ ID NO: 210 - RNA, 23 nt, Artificial sequence
  Synthetic
uccgguccagagcuguuccucau

SEQ ID NO: 211 - RNA, 23 nt, Artificial sequence
  Synthetic
uucggcccagaggugcucgucau

SEQ ID NO: 212 - RNA, 23 nt, Artificial sequence
  Synthetic
uucggcccggagcugcugcucau

SEQ ID NO: 213 - RNA, 23 nt, Artificial sequence
  Synthetic
uucgcuccagagcugcuccucaa

SEQ ID NO: 214 - RNA, 23 nt, Artificial sequence
  Synthetic
guuggcccagaucuucuccucau

SEQ ID NO: 215 - RNA, 23 nt, Artificial sequence
  Synthetic
ugcgguccagagcugcuccucau

SEQ ID NO: 216 - RNA, 23 nt, Artificial sequence
  Synthetic
cucggcccagagcugacccucau

SEQ ID NO: 217 - RNA, 23 nt, Artificial sequence
  Synthetic
uucggcccagagcugcuccuccc

SEQ ID NO: 218 - RNA, 23 nt, Artificial sequence
  Synthetic
cacggcccagagcugcuccucau

SEQ ID NO: 219 - RNA, 23 nt, Artificial sequence
  Synthetic
uacggcccagagcuccuccucau

SEQ ID NO: 220 - RNA, 23 nt, Artificial sequence
  Synthetic
uucggcccagagcuucuucucau

SEQ ID NO: 221 - RNA, 23 nt, Artificial sequence
  Synthetic
uugggcccagagcugcuccucuu

SEQ ID NO: 222 - RNA, 23 nt, Artificial sequence
  Synthetic
uucggcccagagcuccuccuaau

SEQ ID NO: 223 - RNA, 23 nt, Artificial sequence
  Synthetic
uucgacccagagcugcuucucau

SEQ ID NO: 224 - RNA, 23 nt, Artificial sequence
  Synthetic
uucggcccagagcuccuccuuau

SEQ ID NO: 225 - RNA, 23 nt, Artificial sequence
  Synthetic
uucgacccagagcugccccucau

SEQ ID NO: 226 - RNA, 23 nt, Artificial sequence
  Synthetic
cucggcccagagcuucuccucau

SEQ ID NO: 227 - RNA, 23 nt, Artificial sequence
  Synthetic
uucgacccagagcugcuccuccg

SEQ ID NO: 228 - RNA, 23 nt, Artificial sequence
  Synthetic
uugggcccagagcugccccucau

SEQ ID NO: 229 - RNA, 23 nt, Artificial sequence
  Synthetic
uccggcccagagcuccuccucau

SEQ ID NO: 230 - RNA, 23 nt, Artificial sequence
  Synthetic
uccggcccagagcuccuccucau

SEQ ID NO: 231 - RNA, 23 nt, Artificial sequence
  Synthetic
uucggcccagagcuccucuucau

SEQ ID NO: 232 - RNA, 23 nt, Artificial sequence
  Synthetic
uuaggcccagagcuccuccucaa

SEQ ID NO: 233 - RNA, 23 nt, Artificial sequence
  Synthetic
uucgacccagagcugcucaucau

SEQ ID NO: 234 - RNA, 23 nt, Artificial sequence
  Synthetic
gucgccccagagcugcucgucau

SEQ ID NO: 235 - RNA, 23 nt, Artificial sequence
  Synthetic
uucggcccagugcugcuccuuau

SEQ ID NO: 236 - RNA, 23 nt, Artificial sequence
  Synthetic
uucagcccaaagcugcuccucau

SEQ ID NO: 237 - RNA, 23 nt, Artificial sequence
  Synthetic
uucgccccagagcugcuccuaau

SEQ ID NO: 238 - RNA, 23 nt, Artificial sequence
  Synthetic
uucugcccagagcugcucuucau

SEQ ID NO: 239 - RNA, 23 nt, Artificial sequence
  Synthetic
uucgggccagagcuccuccucau

SEQ ID NO: 240 - RNA, 23 nt, Artificial sequence
  Synthetic
uccggcccagagcugcuccuuau

SEQ ID NO: 241 - RNA, 23 nt, Artificial sequence
  Synthetic
ugcggcccagagcugcuccucua

SEQ ID NO: 242 - RNA, 23 nt, Artificial sequence
  Synthetic
uucgacccagagcugcuccuuau

SEQ ID NO: 243 - RNA, 23 nt, Artificial sequence
  Synthetic
uucgacccagagcugauccucau

SEQ ID NO: 244 - RNA, 23 nt, Artificial sequence
  Synthetic
uucgacccagagcugcuccccag

SEQ ID NO: 245 - RNA, 23 nt, Artificial sequence
  Synthetic
uucggcccagagcugcuucuauu

SEQ ID NO: 246 - RNA, 23 nt, Artificial sequence
  Synthetic
gacggcccagagcugcucaucau

SEQ ID NO: 247 - RNA, 23 nt, Artificial sequence
  Synthetic
uucggcccagagcugcuucucua

SEQ ID NO: 248 - RNA, 23 nt, Artificial sequence
  Synthetic
ugcggcccagagcugcuccgcau

SEQ ID NO: 249 - RNA, 23 nt, Artificial sequence
  Synthetic
uucgccccagagcugcuccccau

SEQ ID NO: 250 - RNA, 23 nt, Artificial sequence
  Synthetic
uucggcccagagcuucuccuaau

SEQ ID NO: 251 - RNA, 23 nt, Artificial sequence
  Synthetic
uucuguccagagcugcuccucau

SEQ ID NO: 252 - RNA, 23 nt, Artificial sequence
  Synthetic
uucggcccagagcugccccccau

SEQ ID NO: 253 - RNA, 23 nt, Artificial sequence
  Synthetic
uucuacccagagcugcuccucau

SEQ ID NO: 254 - RNA, 23 nt, Artificial sequence
  Synthetic
uuggguccagagcugcuccucau

SEQ ID NO: 255 - RNA, 23 nt, Artificial sequence
  Synthetic
uucggcccagagcugcuccuacu

SEQ ID NO: 256 - RNA, 23 nt, Artificial sequence
  Synthetic
uucgucccagagcugccccucau

SEQ ID NO: 257 - RNA, 23 nt, Artificial sequence
  Synthetic
uucggcccagagcuacucaucau

SEQ ID NO: 258 - RNA, 23 nt, Artificial sequence
  Synthetic
uucgacccagagcugcuccucaa

SEQ ID NO: 259 - RNA, 23 nt, Artificial sequence
  Synthetic
cucggcccagagcugcuccucuu

SEQ ID NO: 260 - RNA, 23 nt, Artificial sequence
  Synthetic
uucggcgcagggcugcuccucau

SEQ ID NO: 261 - RNA, 23 nt, Artificial sequence
  Synthetic
uucggcccagaucuucuccuuau

SEQ ID NO: 262 - RNA, 23 nt, Artificial sequence
  Synthetic
aucgacccagagcugcuccucau

SEQ ID NO: 263 - RNA, 23 nt, Artificial sequence
  Synthetic
uacgacccagagcugcuccucaa

SEQ ID NO: 264 - RNA, 23 nt, Artificial sequence
  Synthetic
ugcgacccagagcugcuccucau

SEQ ID NO: 265 - RNA, 23 nt, Artificial sequence
  Synthetic
uucggcccagagcuccuccucac

SEQ ID NO: 266 - RNA, 23 nt, Artificial sequence
  Synthetic
gucgguccagaggugcuccucau

SEQ ID NO: 267 - RNA, 23 nt, Artificial sequence
  Synthetic
uucggcccagagcugcuucucag

SEQ ID NO: 268 - RNA, 23 nt, Artificial sequence
  Synthetic
uucgacccagagcugcuaaucau

SEQ ID NO: 269 - RNA, 23 nt, Artificial sequence
  Synthetic
uuagguccagagcugcuacucau

SEQ ID NO: 270 - RNA, 23 nt, Artificial sequence
  Synthetic
uuaggcccagagcuccuccucau

SEQ ID NO: 271 - RNA, 23 nt, Artificial sequence
  Synthetic
uugggcccagagcugcuccucaa

SEQ ID NO: 272 - RNA, 23 nt, Artificial sequence
  Synthetic
uucgacccagagcugcuccucau

SEQ ID NO: 273 - RNA, 23 nt, Artificial sequence
  Synthetic
uucgacccagagcugcuacucau

SEQ ID NO: 274 - RNA, 23 nt, Artificial sequence
  Synthetic
uucggcccagagcugguccacau

SEQ ID NO: 275 - RNA, 23 nt, Artificial sequence
  Synthetic
cucggcccagagcugauccucau

SEQ ID NO: 276 - RNA, 23 nt, Artificial sequence
  Synthetic
uucggcccagagcuccugcucau

SEQ ID NO: 277 - RNA, 23 nt, Artificial sequence
  Synthetic
uucggcccagagcugcuccccuu

SEQ ID NO: 278 - RNA, 23 nt, Artificial sequence
  Synthetic
uucggcccagagcuccuccgcau

SEQ ID NO: 279 - RNA, 23 nt, Artificial sequence
  Synthetic
ugcggcccagagcugcuucucac

SEQ ID NO: 280 - RNA, 23 nt, Artificial sequence
  Synthetic
uucggcccagagcuccuccacau

SEQ ID NO: 281 - RNA, 23 nt, Artificial sequence
  Synthetic
uucggcccagagcugcuuuucau

SEQ ID NO: 282 - RNA, 23 nt, Artificial sequence
  Synthetic
uucgacccagagcuccuccucaa

SEQ ID NO: 283 - RNA, 23 nt, Artificial sequence
  Synthetic
gacgacccagagcugcuccucau

SEQ ID NO: 284 - RNA, 23 nt, Artificial sequence
  Synthetic
ugcgacccagagcugcuccccau

SEQ ID NO: 285 - RNA, 23 nt, Artificial sequence
  Synthetic
uucggcccagagcuccuccucau

SEQ ID NO: 286 - RNA, 23 nt, Artificial sequence
  Synthetic
uucugcccagagcugcucgucau

SEQ ID NO: 287 - RNA, 23 nt, Artificial sequence
  Synthetic
ugcggcccagaacuccuccucau

SEQ ID NO: 288 - RNA, 23 nt, Artificial sequence
  Synthetic
uucggcccaaagcugcuccccau

SEQ ID NO: 289 - RNA, 23 nt, Artificial sequence
  Synthetic
uucgguccagagcuccuccucau

SEQ ID NO: 290 - RNA, 23 nt, Artificial sequence
  Synthetic
uucgucccagagcugcuccucag

SEQ ID NO: 291 - RNA, 23 nt, Artificial sequence
  Synthetic
uucggcccagaucugcuccuccu

SEQ ID NO: 292 - RNA, 23 nt, Artificial sequence
  Synthetic
uucggcccagagcaccuccucau

SEQ ID NO: 293 - RNA, 23 nt, Artificial sequence
  Synthetic
uucggcgcagaggugcuccucau

SEQ ID NO: 294 - RNA, 23 nt, Artificial sequence
  Synthetic
uucggcccagagcuuacccucuu

SEQ ID NO: 295 - RNA, 23 nt, Artificial sequence
  Synthetic
uucggcccagagcugaucuucau

SEQ ID NO: 296 - RNA, 23 nt, Artificial sequence
  Synthetic
uuaugcccagagcugcuccucau

SEQ ID NO: 297 - RNA, 23 nt, Artificial sequence
  Synthetic
uucggcccagaggugcucuucau

SEQ ID NO: 298 - RNA, 23 nt, Artificial sequence
  Synthetic
cucggcccagagcuccuccucau

SEQ ID NO: 299 - RNA, 23 nt, Artificial sequence
  Synthetic
uucggcccagagcuccgccucau

SEQ ID NO: 300 - RNA, 23 nt, Artificial sequence
  Synthetic
uucggcccagagguccuccucuu

SEQ ID NO: 301 - RNA, 23 nt, Artificial sequence
  Synthetic
uucggcccagaggugcuccucuu

SEQ ID NO: 302 - RNA, 23 nt, Artificial sequence
  Synthetic
ugcggcccagagcugcuccucau

SEQ ID NO: 303 - RNA, 23 nt, Artificial sequence
  Synthetic
gacgcauccgcccagagcuacuc

SEQ ID NO: 304 - RNA, 23 nt, Artificial sequence
  Synthetic
cacacuucgggccagagcaacuc

SEQ ID NO: 305 - RNA, 23 nt, Artificial sequence
  Synthetic
gacgcuugggcccagagcugcuc

SEQ ID NO: 306 - RNA, 23 nt, Artificial sequence
  Synthetic
gacgcugcggcccagagcugcuc

SEQ ID NO: 307 - RNA, 23 nt, Artificial sequence
  Synthetic
ggcaccucggcccagagcugcuc

SEQ ID NO: 308 - RNA, 23 nt, Artificial sequence
  Synthetic
cacacuucugcccagagcugcuc

SEQ ID NO: 309 - RNA, 23 nt, Artificial sequence
  Synthetic
gaggcuucggcccagagcugcuc

SEQ ID NO: 310 - RNA, 23 nt, Artificial sequence
  Synthetic
gacgcuuccgcccagagcugcuc

SEQ ID NO: 311 - RNA, 23 nt, Artificial sequence
  Synthetic
gagccuucgacccagagcugcuc

SEQ ID NO: 312 - RNA, 23 nt, Artificial sequence
  Synthetic
gacacuucgguccagagcuccuc

SEQ ID NO: 313 - RNA, 23 nt, Artificial sequence
  Synthetic
gacucuucggcccagggcuccuc

SEQ ID NO: 314 - RNA, 23 nt, Artificial sequence
  Synthetic
gacgcuucgacccagagcugcuc

SEQ ID NO: 315 - RNA, 23 nt, Artificial sequence
  Synthetic
gucaccucgacccagagcugcuc

SEQ ID NO: 316 - RNA, 23 nt, Artificial sequence
  Synthetic
gacccuuugacccagagcugcuc

SEQ ID NO: 317 - RNA, 23 nt, Artificial sequence
  Synthetic
gacccuucggcccagagcuccuc

SEQ ID NO: 318 - RNA, 23 nt, Artificial sequence
  Synthetic
ggcacuucgacccagagcugcuc

SEQ ID NO: 319 - RNA, 23 nt, Artificial sequence
  Synthetic
gccacuucgacccagagcugcuc

SEQ ID NO: 320 - RNA, 23 nt, Artificial sequence
  Synthetic
gacgcuucagcccagagcugcuc

SEQ ID NO: 321 - RNA, 23 nt, Artificial sequence
  Synthetic
gacaauucgguccagagcugcuc

SEQ ID NO: 322 - RNA, 23 nt, Artificial sequence
  Synthetic
gacgcuucggcccagugcugcuc

SEQ ID NO: 323 - RNA, 23 nt, Artificial sequence
  Synthetic
ggcacuucgucccagagcugcuc

SEQ ID NO: 324 - RNA, 23 nt, Artificial sequence
  Synthetic
gaaacaucgacccagagcugcuc

SEQ ID NO: 325 - RNA, 23 nt, Artificial sequence
  Synthetic
gacauuucgacccagagcugcuc

SEQ ID NO: 326 - RNA, 23 nt, Artificial sequence
  Synthetic
ggcgcuucggcccagagcugcuc

SEQ ID NO: 327 - RNA, 23 nt, Artificial sequence
  Synthetic
gacacuucgucccagagcugcuu

SEQ ID NO: 328 - RNA, 23 nt, Artificial sequence
  Synthetic
gacgccucggcccagagcugcuc

SEQ ID NO: 329 - RNA, 23 nt, Artificial sequence
  Synthetic
gacgcuucugcccagagcugcuc

SEQ ID NO: 330 - RNA, 23 nt, Artificial sequence
  Synthetic
gagauuucggcccagagcugcuc

SEQ ID NO: 331 - RNA, 23 nt, Artificial sequence
  Synthetic
gacacuugggcccagagcuccuc

SEQ ID NO: 332 - RNA, 23 nt, Artificial sequence
  Synthetic
gcgacuucgacccagagcugcuc

SEQ ID NO: 333 - RNA, 23 nt, Artificial sequence
  Synthetic
ggcaguucggcccagagcugcuc

SEQ ID NO: 334 - RNA, 23 nt, Artificial sequence
  Synthetic
uauacuacggcccagagcugcuc

SEQ ID NO: 335 - RNA, 23 nt, Artificial sequence
  Synthetic
gagacuucgacccagagcugcuc

SEQ ID NO: 336 - RNA, 23 nt, Artificial sequence
  Synthetic
gacacugcggcgcagagcugcuc

SEQ ID NO: 337 - RNA, 23 nt, Artificial sequence
  Synthetic
gucacuacggcccagagcugcuc

SEQ ID NO: 338 - RNA, 23 nt, Artificial sequence
  Synthetic
gacacaacggcccagagcugcuc

SEQ ID NO: 339 - RNA, 23 nt, Artificial sequence
  Synthetic
gacauaucgacccagagcugcuc

SEQ ID NO: 340 - RNA, 23 nt, Artificial sequence
  Synthetic
gauccuucggcccagagcugcuc

SEQ ID NO: 341 - RNA, 23 nt, Artificial sequence
  Synthetic
gaaacuucgacccagagcugcuc

SEQ ID NO: 342 - RNA, 23 nt, Artificial sequence
  Synthetic
gaaaauucgguccagagcugcuc

SEQ ID NO: 343 - RNA, 23 nt, Artificial sequence
  Synthetic
ggcacuuccgcccagagcugcuc

SEQ ID NO: 344 - RNA, 23 nt, Artificial sequence
  Synthetic
gacccuucggcccagagcugguu

SEQ ID NO: 345 - RNA, 23 nt, Artificial sequence
  Synthetic
gaagcuucggcccagagcugcuc

SEQ ID NO: 346 - RNA, 23 nt, Artificial sequence
  Synthetic
gauacuucgacccagagcugcuc

SEQ ID NO: 347 - RNA, 23 nt, Artificial sequence
  Synthetic
gggacuucggcccagagcugcuc

SEQ ID NO: 348 - RNA, 23 nt, Artificial sequence
  Synthetic
gacgcuucggcccagagaugcuc

SEQ ID NO: 349 - RNA, 23 nt, Artificial sequence
  Synthetic
gacacaucgacccagagcugcuc

SEQ ID NO: 350 - RNA, 23 nt, Artificial sequence
  Synthetic
gacagaucggcccagagcugcuc

SEQ ID NO: 351 - RNA, 23 nt, Artificial sequence
  Synthetic
gugacuucggcccagagcugcuc

SEQ ID NO: 352 - RNA, 23 nt, Artificial sequence
  Synthetic
ggcacugcggcccagagcugcuc

SEQ ID NO: 353 - RNA, 23 nt, Artificial sequence
  Synthetic
gacgcuuaggcccagagcugcuc

SEQ ID NO: 354 - RNA, 23 nt, Artificial sequence
  Synthetic
gacgcuucgguccagagcugcuc

SEQ ID NO: 355 - RNA, 23 nt, Artificial sequence
  Synthetic
gacgcuacggcccagagcugcuc

SEQ ID NO: 356 - RNA, 23 nt, Artificial sequence
  Synthetic
gauauuucgacccagagcugcuc

SEQ ID NO: 357 - RNA, 23 nt, Artificial sequence
  Synthetic
gacucuucgacccagagcugcuc

SEQ ID NO: 358 - RNA, 23 nt, Artificial sequence
  Synthetic
gacacgacggcccagagcugcuc

SEQ ID NO: 359 - RNA, 23 nt, Artificial sequence
  Synthetic
uacacuuugacccagagcugcuc

SEQ ID NO: 360 - RNA, 23 nt, Artificial sequence
  Synthetic
gacacuucgacccagagcuguuc

SEQ ID NO: 361 - RNA, 23 nt, Artificial sequence
  Synthetic
gagacugcggcccagagcugcuc

SEQ ID NO: 362 - RNA, 23 nt, Artificial sequence
  Synthetic
gacauuuugacccagagcugcuc

SEQ ID NO: 363 - RNA, 23 nt, Artificial sequence
  Synthetic
gucacuucgacccagagcugcuc

SEQ ID NO: 364 - RNA, 23 nt, Artificial sequence
  Synthetic
ggaacuugggcccagagcugcuc

SEQ ID NO: 365 - RNA, 23 nt, Artificial sequence
  Synthetic
gacgcuucggcccagagcugcuc

SEQ ID NO: 366 - RNA, 23 nt, Artificial sequence
  Synthetic
gacacuacgacccagagcugcuc

SEQ ID NO: 367 - RNA, 23 nt, Artificial sequence
  Synthetic
gacacagcggcccagagcugcuc

SEQ ID NO: 368 - RNA, 23 nt, Artificial sequence
  Synthetic
gacacuucgccccggagcugcuc

SEQ ID NO: 369 - RNA, 23 nt, Artificial sequence
  Synthetic
gacacuucgacccagagcugcuc

SEQ ID NO: 370 - RNA, 23 nt, Artificial sequence
  Synthetic
gucacuugggcccagagcugcuc

SEQ ID NO: 371 - RNA, 23 nt, Artificial sequence
  Synthetic
gacgcuucggaccagagcugcuc

SEQ ID NO: 372 - RNA, 23 nt, Artificial sequence
  Synthetic
gacaguucgacccagagcugcuc

SEQ ID NO: 373 - RNA, 23 nt, Artificial sequence
  Synthetic
gagccuucggcccagagcugcuc

SEQ ID NO: 374 - RNA, 23 nt, Artificial sequence
  Synthetic
gacauuucgccccagagcugcuc

SEQ ID NO: 375 - RNA, 23 nt, Artificial sequence
  Synthetic
gacacuugcacccagagcugcuc

SEQ ID NO: 376 - RNA, 23 nt, Artificial sequence
  Synthetic
gacaguccggcccagagcugcuc

SEQ ID NO: 377 - RNA, 23 nt, Artificial sequence
  Synthetic
gacauugcggcccagagcugcuc

SEQ ID NO: 378 - RNA, 23 nt, Artificial sequence
  Synthetic
gaaccuucggcccagagcugcuc

SEQ ID NO: 379 - RNA, 23 nt, Artificial sequence
  Synthetic
gagacuugggcccagagcugcuc

SEQ ID NO: 380 - RNA, 23 nt, Artificial sequence
  Synthetic
gccacuucggcccagaguugcgc

SEQ ID NO: 381 - RNA, 23 nt, Artificial sequence
  Synthetic
gacgcuucgucccagagcugcuc

SEQ ID NO: 382 - RNA, 23 nt, Artificial sequence
  Synthetic
ugcacuucggcccagagcugcuc

SEQ ID NO: 383 - RNA, 23 nt, Artificial sequence
  Synthetic
gagacgucggcccagagcugcuc

SEQ ID NO: 384 - RNA, 23 nt, Artificial sequence
  Synthetic
gauacuucugcccagagcugcuc

SEQ ID NO: 385 - RNA, 23 nt, Artificial sequence
  Synthetic
gacauuuggguccagagcugcuc

SEQ ID NO: 386 - RNA, 23 nt, Artificial sequence
  Synthetic
ugcacuucggcccagagcuucuc

SEQ ID NO: 387 - RNA, 23 nt, Artificial sequence
  Synthetic
gacgcaucggcccagagcugcuc

SEQ ID NO: 388 - RNA, 23 nt, Artificial sequence
  Synthetic
gaaacuucgguccagagcugcuc

SEQ ID NO: 389 - RNA, 23 nt, Artificial sequence
  Synthetic
gacacugggguccagagcugcuc

SEQ ID NO: 390 - RNA, 23 nt, Artificial sequence
  Synthetic
gacacugcggcccagagcugcuu

SEQ ID NO: 391 - RNA, 23 nt, Artificial sequence
  Synthetic
gucacuuugacccagagcugcuc

SEQ ID NO: 392 - RNA, 23 nt, Artificial sequence
  Synthetic
gacacugcggcccagagcugcug

SEQ ID NO: 393 - RNA, 23 nt, Artificial sequence
  Synthetic
gacucuucgauccagagcugcuc

SEQ ID NO: 394 - RNA, 23 nt, Artificial sequence
  Synthetic
gagacaucggcccagagcugcuc

SEQ ID NO: 395 - RNA, 23 nt, Artificial sequence
  Synthetic
gaaacuuagacccagagcugcuc

SEQ ID NO: 396 - RNA, 23 nt, Artificial sequence
  Synthetic
gaccagucggcccagagcugcuc

SEQ ID NO: 397 - RNA, 23 nt, Artificial sequence
  Synthetic
gccacugcggcccagagcugcuc

SEQ ID NO: 398 - RNA, 23 nt, Artificial sequence
  Synthetic
gacacuccgacccagagcugcuc

SEQ ID NO: 399 - RNA, 23 nt, Artificial sequence
  Synthetic
gacacaucgccccagagcugcuc

SEQ ID NO: 400 - RNA, 23 nt, Artificial sequence
  Synthetic
uacacuuccgcccagagcugcuc

SEQ ID NO: 401 - RNA, 23 nt, Artificial sequence
  Synthetic
gacaauuccgcccagaggugcuc

SEQ ID NO: 402 - RNA, 23 nt, Artificial sequence
  Synthetic
gcaacuucggcccagagcugcuc

SEQ ID NO: 403 - RNA, 23 nt, Artificial sequence
  Synthetic
gacauuucgacccagagcugcuu

SEQ ID NO: 404 - RNA, 23 nt, Artificial sequence
  Synthetic
gacgcuacgacccagagcugcuc

SEQ ID NO: 405 - RNA, 23 nt, Artificial sequence
  Synthetic
gagacuucggcccagagcugcgc

SEQ ID NO: 406 - RNA, 23 nt, Artificial sequence
  Synthetic
gacacugcgacccagagcuguuc

SEQ ID NO: 407 - RNA, 23 nt, Artificial sequence
  Synthetic
gagacuucgacccagagcuguuc

SEQ ID NO: 408 - RNA, 23 nt, Artificial sequence
  Synthetic
gacgcuucggcccagagcuacuc

SEQ ID NO: 409 - RNA, 23 nt, Artificial sequence
  Synthetic
gagacuucggcccagagcugcua

SEQ ID NO: 410 - RNA, 23 nt, Artificial sequence
  Synthetic
gacacaucggcccagaucuccuc

SEQ ID NO: 411 - RNA, 23 nt, Artificial sequence
  Synthetic
ggcacaucggcccagagcugcuc

SEQ ID NO: 412 - RNA, 23 nt, Artificial sequence
  Synthetic
gagacuucgccccagagcugcuc

SEQ ID NO: 413 - RNA, 23 nt, Artificial sequence
  Synthetic
gacacuacguuccagagcugcuc

SEQ ID NO: 414 - RNA, 23 nt, Artificial sequence
  Synthetic
gccacuucggcccagagcuccuc

SEQ ID NO: 415 - RNA, 23 nt, Artificial sequence
  Synthetic
gccacaucggcccagagcugcuc

SEQ ID NO: 416 - RNA, 23 nt, Artificial sequence
  Synthetic
gccacuucgacccagagcugguc

SEQ ID NO: 417 - RNA, 23 nt, Artificial sequence
  Synthetic
gacacuuugacccagagcugcuc

SEQ ID NO: 418 - RNA, 23 nt, Artificial sequence
  Synthetic
ggcacuucggcccagagcugccc

SEQ ID NO: 419 - RNA, 23 nt, Artificial sequence
  Synthetic
gacaauucgacccagagcugcuc

SEQ ID NO: 420 - RNA, 23 nt, Artificial sequence
  Synthetic
ggcacuucgacccagagcugccc

SEQ ID NO: 421 - RNA, 23 nt, Artificial sequence
  Synthetic
uacacugcggcccagagcugcuc

SEQ ID NO: 422 - RNA, 23 nt, Artificial sequence
  Synthetic
gacacaucggcccagagcuccuc

SEQ ID NO: 423 - RNA, 23 nt, Artificial sequence
  Synthetic
gacuuuucugcccagagcuacuc

SEQ ID NO: 424 - RNA, 23 nt, Artificial sequence
  Synthetic
gacccuacgguccagagcugcuc

SEQ ID NO: 425 - RNA, 23 nt, Artificial sequence
  Synthetic
gucaguucgacccagagcugcuc

SEQ ID NO: 426 - RNA, 23 nt, Artificial sequence
  Synthetic
gacacugcggcccagagcugcuc

SEQ ID NO: 427 - RNA, 23 nt, Artificial sequence
  Synthetic
gacaguucgucccagagcugcuc

SEQ ID NO: 428 - RNA, 23 nt, Artificial sequence
  Synthetic
gacaugucggcccagagcugcuc

SEQ ID NO: 429 - RNA, 23 nt, Artificial sequence
  Synthetic
gacacuucgacccagagcugguc

SEQ ID NO: 430 - RNA, 23 nt, Artificial sequence
  Synthetic
gagaauucuccccagagcugcuc

SEQ ID NO: 431 - RNA, 23 nt, Artificial sequence
  Synthetic
gccacgucugcccagagcugcuc

SEQ ID NO: 432 - RNA, 23 nt, Artificial sequence
  Synthetic
gacacgucgccccagagcugcuc

SEQ ID NO: 433 - RNA, 23 nt, Artificial sequence
  Synthetic
gacaguucgguccagagcugcuc

SEQ ID NO: 434 - RNA, 23 nt, Artificial sequence
  Synthetic
gucauuucggcccagagcugcuc

SEQ ID NO: 435 - RNA, 23 nt, Artificial sequence
  Synthetic
gacacuucggcccagaucuccuc

SEQ ID NO: 436 - RNA, 23 nt, Artificial sequence
  Synthetic
gagacuucggcccagagcugcuc

SEQ ID NO: 437 - RNA, 23 nt, Artificial sequence
  Synthetic
gacacuacggcccagagcuccuc

SEQ ID NO: 438 - RNA, 23 nt, Artificial sequence
  Synthetic
gagacuucggcccagagcugcuu

SEQ ID NO: 439 - RNA, 23 nt, Artificial sequence
  Synthetic
gacacuucguuccagagcuguuc

SEQ ID NO: 440 - RNA, 23 nt, Artificial sequence
  Synthetic
gaaacuucggcccagagcuccuc

SEQ ID NO: 441 - RNA, 23 nt, Artificial sequence
  Synthetic
gacaggucggcccagagcuucuc

SEQ ID NO: 442 - RNA, 23 nt, Artificial sequence
  Synthetic
gacacugcgccccagagcugcuc

SEQ ID NO: 443 - RNA, 23 nt, Artificial sequence
  Synthetic
gacuauucggcccagagcugcuc

SEQ ID NO: 444 - RNA, 23 nt, Artificial sequence
  Synthetic
gacccuacgacccagagcugauc

SEQ ID NO: 445 - RNA, 23 nt, Artificial sequence
  Synthetic
ggcauuucggcccagagcugcuc

SEQ ID NO: 446 - RNA, 23 nt, Artificial sequence
  Synthetic
gacgguuccgcccagagcugcuc

SEQ ID NO: 447 - RNA, 23 nt, Artificial sequence
  Synthetic
gacucuacggcccagagcugcuc

SEQ ID NO: 448 - RNA, 23 nt, Artificial sequence
  Synthetic
ggcacuucggcccagagcuccuc

SEQ ID NO: 449 - RNA, 23 nt, Artificial sequence
  Synthetic
gucaguucggcccagagcugcuc

SEQ ID NO: 450 - RNA, 23 nt, Artificial sequence
  Synthetic
gauacuucggcccagagcugcuc

SEQ ID NO: 451 - RNA, 23 nt, Artificial sequence
  Synthetic
gacccuucgacccagagcugcuc

SEQ ID NO: 452 - RNA, 23 nt, Artificial sequence
  Synthetic
gagacuucgacccagagcugccc

SEQ ID NO: 453 - RNA, 23 nt, Artificial sequence
  Synthetic
gauacuucgccccagagcugcuc

SEQ ID NO: 454 - RNA, 23 nt, Artificial sequence
  Synthetic
gaaacuucgccccagagcugcuc

SEQ ID NO: 455 - RNA, 23 nt, Artificial sequence
  Synthetic
uacgcuucggcccagagcugcuc

SEQ ID NO: 456 - RNA, 23 nt, Artificial sequence
  Synthetic
gacuguucggcccagagcugcuc

SEQ ID NO: 457 - RNA, 23 nt, Artificial sequence
  Synthetic
uacacuucgacccagagcugcuc

SEQ ID NO: 458 - RNA, 23 nt, Artificial sequence
  Synthetic
gggacuuaggcccagagcugcuc

SEQ ID NO: 459 - RNA, 23 nt, Artificial sequence
  Synthetic
gacagaucgguccagagcugcuc

SEQ ID NO: 460 - RNA, 23 nt, Artificial sequence
  Synthetic
gauacagcggcccagagcugcuc

SEQ ID NO: 461 - RNA, 23 nt, Artificial sequence
  Synthetic
ggcacuugggcccagagcugcuc

SEQ ID NO: 462 - RNA, 23 nt, Artificial sequence
  Synthetic
gacacugcgacccaaagcugcuc

SEQ ID NO: 463 - RNA, 23 nt, Artificial sequence
  Synthetic
gacaucuccgcccagagcugcuc

SEQ ID NO: 464 - RNA, 23 nt, Artificial sequence
  Synthetic
gacauuuccgcccagagcugcuc

SEQ ID NO: 465 - RNA, 23 nt, Artificial sequence
  Synthetic
gacacugcgacccagagcugcuc

SEQ ID NO: 466 - RNA, 23 nt, Artificial sequence
  Synthetic
gacacuucgacccagagcuacuc

SEQ ID NO: 467 - RNA, 23 nt, Artificial sequence
  Synthetic
gauaguccggcccagagcugcuc

SEQ ID NO: 468 - RNA, 23 nt, Artificial sequence
  Synthetic
gacacaucgacccagagcugguc

SEQ ID NO: 469 - RNA, 23 nt, Artificial sequence
  Synthetic
gacacgucggcccagaguugcug

SEQ ID NO: 470 - RNA, 23 nt, Artificial sequence
  Synthetic
gacacgaccgcccagagcugcuc

SEQ ID NO: 471 - RNA, 23 nt, Artificial sequence
  Synthetic
gagauuccgacccagagcuguua

SEQ ID NO: 472 - RNA, 23 nt, Artificial sequence
  Synthetic
gccacuucggcccagagcugcuc

SEQ ID NO: 473 - RNA, 23 nt, Artificial sequence
  Synthetic
gagacuacgguccagagcuccuc

SEQ ID NO: 474 - RNA, 23 nt, Artificial sequence
  Synthetic
gacgcuucggcccaaagcugcuc

SEQ ID NO: 475 - RNA, 23 nt, Artificial sequence
  Synthetic
gccacuucgguccagagcugcuc

SEQ ID NO: 476 - RNA, 23 nt, Artificial sequence
  Synthetic
gucucuucggcccagagcugcuc

SEQ ID NO: 477 - RNA, 23 nt, Artificial sequence
  Synthetic
gcgacuucgucccagagcugcac

SEQ ID NO: 478 - RNA, 23 nt, Artificial sequence
  Synthetic
ggcacuucgccccagagcugcuc

SEQ ID NO: 479 - RNA, 23 nt, Artificial sequence
  Synthetic
guuacuucggcccagagcugcuc

SEQ ID NO: 480 - RNA, 23 nt, Artificial sequence
  Synthetic
ggaacuucggcccagagcugcuc

SEQ ID NO: 481 - RNA, 23 nt, Artificial sequence
  Synthetic
gacacuucggcccagagcuccuc